(19) United States
(12) Patent Application Publication
(10) Pub. No.: US 2019/0192429 A1
(43) Pub. Date: Jun. 27, 2019

(54) SUBLINGUAL FORMULATIONS COMPRISING CANNABIS RESIN, METHODS FOR MAKING SAME AND USES THEREOF

(71) Applicant: CannScience Innovations Inc., Toronto (CA)

(72) Inventors: Lakshmi Premakanth KOTRA, Toronto (CA); Melissa Maureen LEWIS, Bolton (CA); Ewa WASILEWSKI, Mississauga (CA); Har GROVER, Toronto (CA); Ashraf M. ABDELKHALIK, Hamilton (CA)

(21) Appl. No.: 16/230,231

(22) Filed: Dec. 21, 2018

Related U.S. Application Data
(60) Provisional application No. 62/610,706, filed on Dec. 27, 2017.
(60) Provisional application No. 62/694,243, filed on Jul. 5, 2018.

(51) Int. Cl. A61K 9/00; A61K 31/352; A61K 47/26; A61K 47/38; A61K 47/36; A61K 47/02; A61K 47/32

(52) U.S. Cl. CPC A61K 9/0056; A61K 31/352; A61K 47/26; A61K 47/32; A61K 47/36; A61K 47/02; A61K 47/38

(57) ABSTRACT

The disclosure relates to solid formulations comprising decarboxylated cannabis resin. The disclosure provides rapidly disintegrating sublingual tablet formulations comprising decarboxylated cannabis resin, and methods for making and using same. The formulations provided herein may be useful as pharmaceutical and/or natural health products for the treatment or amelioration of various symptoms, disorders and/or diseases.

### CROSS REFERENCE TO RELATED APPLICATIONS

The current application claims the benefit of priority to U.S. Provisional Application Ser. No. 62/694,243, filed on Jul. 5, 2018, and U.S. Provisional Application Ser. No. 62/610,706, filed on Dec. 27, 2017, which is hereby incorporated by reference in its entirety.

### FIELD OF THE DISCLOSURE

The present disclosure relates to solid formulations comprising cannabis resin. In one embodiment, the present disclosure provides sublingual tablet formulations comprising cannabis resin, and methods for making and using same.

### BACKGROUND OF THE DISCLOSURE

Cannabis spp. plants produce a highly complex mixture of compounds, with up to 568 unique compounds identified to date (Pertwee, R. G. e., Handbook of cannabis. Oxford University Press: Oxford, 2014). Two classes of compounds, cannabinoids and terpenes, account for a significant portion of these molecules, with more than 100 types of each having been identified in Cannabis spp. plants (Aizpurua-Olaizola, O.; Soydaner, U.; Ozturk, E.; Schibano, D.; Simsir, Y.; Navarro, P.; Etxebarria, N.; Usobiaga, A. Evolution of the Cannabinoid and Terpene Content during the Growth of Cannabis sativa Plants from Different Chemotypes. Journal of Natural Products 2016, 79, 324-331; incorporated herein by reference). Cannabis spp. plants also produce other compounds such as fatty acids, chlorophyll, and flavonoids.

Among cannabinoids, the compounds tetrahydrocannabinol (THC) and cannabidiol (CBD) are the most dominant as well as most studied with respect to their biological activity and therapeutic potential. Much of the pharmaceutical research has focused on cannabinoids in purified form. However, there is a growing body of research regarding synergistic effects, buffering effects and antagonistic effects (sometimes referred to together as “entourage effects”) between the various cannabinoid compounds, terpene compounds and other compounds found in Cannabis spp. plants (Russo, E. B. Taming THC: potential cannabis synergy and phytocannabinoid-terpenoid entourage effects. British Journal of Pharmacology 2011, 163, 1344-1364; incorporated herein by reference). Accordingly, there is an increased interest in therapeutics which reflect more of the molecular complexity of the original Cannabis plant, such as cannabis resins.

There is also a need for therapeutic forms which can be easily administered. Cannabinoids may be converted to their therapeutically active forms through decarboxylation. To date the simplest method of decarboxylation is by heating, such as by smoking or vaping. However, some people are unwilling to use cannabis if it has to be smoked or vaped due to negative perceptions of smoking and vaping, while others may be physically unable to do so, especially in the palliative care context. Accordingly, there is a need for an improved dosage form, such as a rapidly disintegrating sublingual tablet, which can be easily administered by a person or their caregiver, without further heating.

### SUMMARY OF THE DISCLOSURE

The present disclosure relates to solid formulations comprising decarboxylated cannabis resin. In particular, the present disclosure provides rapidly disintegrating sublingual tablet formulations comprising decarboxylated cannabis resin, and methods for making and using same. The formulations provided herein may be useful as pharmaceutical and/or natural health products for the treatment and amelioration of various symptoms, disorders and/or diseases.

<1> A method for making a rapidly disintegrating sublingual tablet from decarboxylated cannabis resin, wherein the decarboxylated cannabis resin comprises cannabinoids which are at least 50% decarboxylated, the method comprising:
(a) dissolving the decarboxylated cannabis resin in a pharmaceutically acceptable organic solvent to produce a first solution;
(b) dissolving mannitol in a pharmaceutically acceptable polar solvent to produce a second solution;
(c) mixing the first solution of (a) and the second solution of (b);
(d) substantially removing the solvents from the mixture of (c) to produce a powder;
(e) adding one or more pharmaceutically acceptable excipients to the powder of (d) to produce a mixture, solid solution or solid suspension; wherein at least one of the pharmaceutically acceptable excipients is a disintegrant;
(f) triturating or mixing the mixture, solid solution or solid suspension of (e) to produce a homogenous mixture, homogeneous solid solution or homogeneous solid suspension; and
(g) compressing the homogenous mixture, homogeneous solid solution or homogeneous solid suspension of (f) into a tablet using a pressure of up to about 1 ton.

<2> The method of <1>, wherein the cannabinoids are at least 55%, at least 60%, at least 65%, at least 70%, at least 75%, at least 80%, at least 85%, at least 90%, at least 95%, at least 96%, at least 97%, at least 98%, at least 99%, or 100% decarboxylated.

<3> The method of <1>, wherein the ratio of cannabis resin to mannitol is about 1:3 to 1:8.

<4> The method of <1>, wherein the organic solvent of (a) is ethanol; wherein the polar solvent of (b) is water; and wherein the disintegrant of (e) is cross-linked polyvinylpyrrolidone and/or croscarmellose sodium.

<5> The method of <4>, wherein the one or more pharmaceutically acceptable excipients comprise magnesium stearate, cross-linked polyvinylpyrrolidone, and optionally microcrystalline cellulose.

<6> The method of <1>, wherein in step (d) the solvents are substantially removed by lyophilization, spray drying, or fluid bed drying.

<7> The method of <1>, wherein in step (g) the pressure is from about 0.05 ton to about 0.6 ton or from about 0.1 to about 0.4 ton.

<8> The method of <1>, wherein the decarboxylated cannabis resin comprises 0 to 95% Δ⁹-tetrahydrocannabinol (Δ⁹-THC) and 0-95% cannabidiol (CBD) including combinations thereof.

<9> The method of <1>, wherein the resin is about 2-18 wt % of the tablet, the mannitol is about 40-80 wt % of the tablet, the disintegrant is about 10-50 wt % of the tablet, and optionally other excipients are about 0-25 wt % of the tablet.

<10> The method of <9>, wherein the resin is about 7-11 wt % of the tablet, the mannitol is about 50-63 wt % of the tablet, the disintegrant is cross-linked polyvinylpyrrolidone and/or croscarmellose sodium and is about 11-18 wt % of the tablet, and the other excipients are magnesium stearate which is about 0.6-1.1 wt % of the tablet, and microcrystalline cellulose which is about 0-20 wt % of the tablet.

<11> The method of <1>, wherein the rapidly disintegrating tablet disintegrates in less than 60 seconds when contacted with a phosphate buffered saline (PBS); and wherein the tablet has a friability of less than 5%.

<12> A rapidly disintegrating sublingual tablet from decarboxylated cannabis resin, comprising cannabinoids which are at least 50% decarboxylated, wherein the tablet further comprises mannitol, a disintegrant, and optionally one or more other pharmaceutically acceptable excipients, wherein the tablet disintegrates in less than 60 seconds when contacted with phosphate buffered saline (PBS) and wherein the tablet has a friability of less than 5%.

<13> The tablet of <12>, wherein the cannabinoids are at least 55%, at least 60%, at least 65%, at least 70%, at least 75%, at least 80%, at least 85%, at least 90%, at least 95%, at least 96%, at least 97%, at least 98%, at least 99%, or 100% decarboxylated.

<14> The tablet of <12>, wherein the ratio of cannabis resin to mannitol is about 1:3 to 1:8.

<15> The tablet of <12>, wherein the disintegrant is cross-linked polyvinylpyrrolidone and/or croscarmellose sodium.

<16> The tablet of <15>, wherein the one or more pharmaceutically acceptable excipients comprise magnesium stearate and optionally microcrystalline cellulose.

<17> The tablet of <12>, wherein the combined total of Δ⁹-tetrahydrocannabinol (Δ⁹-THC) and cannabidiol (CBD) is up to about 18 wt % of the tablet.

<18> The tablet of <12>, wherein the resin is about 2-18 wt % of the tablet, the mannitol is about 40-80 wt % of the tablet, the disintegrant is about 10-50 wt % of the tablet, and optionally the other excipients are about 0-25 wt % of the tablet.

<19> The tablet of <18>, wherein the other excipients are optionally a diluent, a filler, a binding agent, a releasing agent, a lubricant, a flavoring agent, a taste-masking agent, and/or a colorant.

<20> Use of a decarboxylated cannabis resin in the manufacture of a rapidly disintegrating sublingual tablet, wherein the resin comprises decarboxylated cannabinoids from a Cannabis spp. plant, such that Δ9-tetrahydrocannabinolic acid (Δ⁹-THCA) and cannabidiolic acid (CBDA) cannabinoids are each independently 90%-100% decarboxylated to yield Δ⁹-tetrahydrocannabinol (Δ⁹-THC) and cannabidiol (CBD) respectively in the cannabis resin.

Other features and advantages of the disclosure will be apparent from the following detailed description and from the exemplary embodiments.

### BRIEF DESCRIPTION OF THE DRAWINGS

In order that the subject matter may be readily understood, embodiments are illustrated by way of examples in the accompanying drawings, in which:

FIG. 1A shows chemical structures of six major cannabinoids present in cannabis: Δ⁹-THC, Δ⁹-THC-A, Δ⁸-THC, CBN, Δ²-CBD and Δ²-CBD-A;

FIG. 1B shows the cannabinoic acid synthase pathway of cannabigerolic acid (CBGA) to cannabidiolic acid (CBDA), tetrahydrocannabinolic acid (THCA) and cannabichromenic acid (CBCA);

FIG. 2A shows medical cannabis and decarboxylated cannabis resin;

FIG. 2B is a mass spectra of Strain 1 before decarboxylation of medical cannabis;

FIG. 2C is a mass spectra of Strain 1 after decarboxylation of medical cannabis;

FIG. 3 is a block diagram illustrating a process for extracting and decarboxylating cannabinoids from cannabis plant material according to one embodiment of the application;

FIG. 4 shows a standard curve for Δ⁸-THC (selected ion recording (SIR) chromatograms were integrated and the AUC was plotted vs. concentration (ug/mL);

FIG. 5 shows a standard curve for Δ⁹-THC (selected ion recording (SIR) chromatograms were integrated and the AUC was plotted vs. concentration (ug/mL);

FIG. 6 shows a standard curve for THCA (selected ion recording (SIR) chromatograms were integrated and the AUC was plotted vs. concentration (ug/mL);

FIG. 7 shows a standard curve for CBD (selected ion recording (SIR) chromatograms were integrated and the AUC was plotted vs. concentration (ug/mL);

FIG. 8 shows a standard curve for CBDA (selected ion recording (SIR) chromatograms were integrated and the AUC was plotted vs. concentration (ug/mL);

FIG. 9 shows a standard curve for CBN (selected ion recording (SIR) chromatograms were integrated and the AUC was plotted vs. concentration (ug/mL);

FIG. 10 shows a relative amount of Δ⁹-THC and THC-A present in cannabis plant material samples extracted using microwave radiation at various temperatures;

FIG. 11 shows concentrations of various cannabinoids in the cannabis extracts using ethanol, Soxhlet and SFE, without subjecting cannabis to microwave conditions;

FIG. 12 shows concentrations of various cannabinoids in the cannabis extracts using ethanol, Soxhlet and SFE followed by microwave heating;

FIG. 13 shows various cannabinoid concentrations from cannabis after microwave heating only at various temperatures;

FIG. 14 shows various cannabinoid concentrations from cannabis after microwave heating and subsequent SFE extraction at various temperatures;

FIG. 15 shows various cannabinoid concentrations from cannabis after microwave heating and subsequent SFE extraction at various temperatures, where the acidic and neutral form of THC are added together to represent the total THC extracted by each method;

FIG. 16 shows a chromatogram of mass scan in a positive mode (150-500 m/z) of cannabis extract obtained from microwave extraction at 170° C., 15 min;

FIG. 17 shows single ion recording (SIR) (+ve) detection of m/z=315 for cannabis extract obtained from microwave extraction at 170° C., 15 min;

FIG. 18 shows SIR (+ve) detection of m/z=359 for cannabis extract obtained from microwave extraction at 170° C., 15 min;

FIG. 19 shows a chromatogram of a mass scan in a negative mode (150-500 m/z) of cannabis extract obtained from microwave extraction at 170° C., 15 min;

FIG. 20 shows SIR (−ve) detection of m/z=313 for cannabis extract obtained from microwave extraction at 170° C., 15 min;

FIG. 21 shows SIR (−ve) detection of m/z=357 for cannabis extract obtained from microwave extraction at 170° C., 15 min;

FIG. 22 shows a chromatogram of mass scan in a positive mode (150-500 m/z) of cannabis extract obtained from microwave extraction at 130° C., 10 min;

FIG. 23 shows SIR (+ve) detection of m/z=315 for cannabis extract obtained from microwave extraction at 130° C., 10 min;

FIG. 24 shows SIR (+ve) detection of m/z=359 for cannabis extract obtained from microwave extraction at 130° C., 10 min;

FIG. 25 shows a chromatogram of mass scan in a negative mode (150-500 m/z) of cannabis extract obtained from microwave extraction at 130° C., 10 min;

FIG. 26 shows SIR (−ve) detection of m/z=313 for cannabis extract obtained from microwave extraction at 130° C., 10 min;

FIG. 27 shows SIR (−ve) detection of m/z=357 for cannabis extract obtained from microwave extraction at 130° C., 10 min;

FIG. 28A Overview of select signals as seen in the mass spectra before decarboxylation of cannabis extract;

FIG. 28B Overview of select signals as seen in the mass spectra after decarboxylation of cannabis extract;

FIG. 29 shows graphs of physical property measurements of rapidly disintegrating tablet (RDT) formulations over the course of a 40 day stability study at 40° C.±2° C./75%±5% relative humidity in a stability chamber (Caron 7000-10, Benchtop), where CDS-ML-II-66-1 is Formulation 34, CDS-ML-II-66-2 is Formulation 35, and CDS-ML-II-79 is Formulation 43.

FIG. 30A shows a graph of friability and moisture content measurements of rapidly disintegrating tablet (RDT) Formulation 64 over the course of a 6-month stability study at 40° C.±2° C./75%±5% relative humidity in a stability chamber (Caron 7000-10, Benchtop) at 0, 1, 3 and 6 months.

FIG. 30B shows a graph of hardness and disintegration time measurements of rapidly disintegrating tablet (RDT) Formulation 64 over the course of a 6-month stability study at 40° C.±2° C./75%±5% relative humidity in a stability chamber (Caron 7000-10, Benchtop) at 0, 1, 3 and 6 months.

FIG. 31 shows a graph of quantity of cannabinoids Δ⁹-THC, CBD, and CBN of rapidly disintegrating tablet (RDT) Formulation 64 over the course of a 6-month stability study at 40° C.±2° C./75%±5% relative humidity in a stability chamber (Caron 7000-10, Benchtop) at 0, 1, 3 and 6 months.

### DETAILED DESCRIPTION OF THE DISCLOSURE

### Definitions

Unless defined otherwise, all technical and scientific terms used herein have the same meaning as commonly understood by one of ordinary skill in the art to which this invention belongs.

“Active cannabinoid” as used herein, refers to a cannabinoid that has high potency at the corresponding receptor, often with an EC₅₀ 1 μM or less; typically a decarboxylated phytocannabinoid that is in its neutral form such as Δ⁹-THC.

“Cannabinoid” as used herein, refers to a class of diverse chemical compounds that interact with cannabinoid receptors (for example, CB1 and CB2) on the cell surface of neurons and other cell types; the term encompassing both cannabis-derived phytocannabinoid compounds and endogenously-produced endocannabinoid compounds, and those synthetically prepared.

“Cannabis plant” or “Cannabis spp. plant” as used herein, refers to any one or more plant(s) from the Cannabis genus of flowering plants in the family Cannabaceae; including but not limited to Cannabis sativa, Cannabis indica and Cannabis ruderalis, and all subspecies thereof (for example, Cannabis sativa subspecies indica including the variants var. indica and var. kafiristanica); including wild or domesticated type Cannabis plants and also variants thereof; including Cannabis plant chemovars (varieties characterized by their chemical composition) which contain different amounts and/or ratios of the individual cannabinoids, terpenes and/or other compounds; including Cannabis plants which are the result of genetic crosses, self-crosses or hybrids thereof; including female and “feminized” plants (which may produce a higher concentration of cannabinoids), and male plants (which may produce a lower concentration of cannabinoids). As is known to the person skilled in the art, Cannabis spp. includes hemp.

“Cannabis plant material” as used herein, refers to plant material derived directly from one or more Cannabis spp. plants; including live or fresh cannabis plants and dried cannabis plants; including but not limited to trichomes, flower buds, flower bracts, leaves, stalk and any other part of cannabis plant.

“Decarboxylated cannabis resin” as used herein, refers to the hydrophobic, viscous, glue-like substance that is produced by extraction (chemical or physical) and decarboxylation of various parts of a cannabis plant, in particular glandular trichomes of the flower. Such a resin contains predominately (>50%, ideally >90%) decarboxylated cannabinoids, while reflecting at least some of the molecular diversity of the original cannabis plant, including some or all of cannabinoids, terpenes, flavonoids and/or other compounds of interest, some of which may have undergone chemical transformation during the processes used for extraction and decarboxylation. The term excludes predominately (>50%) non-decarboxylated resinous substances derived from cannabis (for example, kief, hash, hashish, etc.).

“Decarboxylation” as used herein, refers to a process of removal of a carboxylic group from a cannabinoid molecule such as Δ⁹-THCA or CBDA (an acid form) to the corresponding neutral form such as Δ⁹-THC and CBD; wherein a carboxyl group is removed from the cannabinoid molecule, and carbon dioxide is released.

“Friability” as used herein, refers to the tendency of a solid substance to break into smaller pieces with handling or contact. Friability testing is a laboratory technique to test the durability of tablets during transit or handling. Friability is calculated as the percentage of weight lost by tablets due to mechanical action during a friability test.

“Hardness” as used herein, refers to tablet hardness as assayed using a laboratory technique to test the breaking point and structural integrity of a tablet under conditions of storage, transportation and handling before usage.

“Inactive cannabinoid” as used herein, refers to a cannabinoid that has poor potency at the corresponding receptor, often with an EC₅₀ greater than 1 μM; typically a cannabinoid that is in its acidic form such as Δ⁹-THCA, a non-decarboxylated cannabinoid.

“Microwaves” as used herein, refer to a form of electromagnetic radiation with wavelengths ranging from one meter to one millimeter; and with frequencies between 300 MHz (100 cm) and 300 GHz (0.1 cm).

“Pharmaceutically acceptable”, as used in connection with raw materials and/or formulations and/or compositions of the present disclosure, refers to molecular entities and other ingredients of such compositions that are physiologically tolerable and do not typically produce untoward reactions when administered to a mammal (for example, a human). Preferably, as used herein, the term “pharmaceutically acceptable” means approved by a regulatory agency of the Federal or a state government or listed in the U.S. Pharmacopeia (USP), National Formulary (NF), or other generally recognized pharmacopeia for use in mammals, and more particularly in humans.

“Rapidly disintegrating tablet” or “RDT” as used herein, refers to a solid dosage form containing a therapeutic substance or active ingredient which disintegrates rapidly in Phosphate Buffer Solution (PBS) and/or saliva or other similar artificial or natural fluids.

“Sublingual” as used herein, refers to the pharmacological route of administration by which a therapeutic substance is placed under the tongue.

### Abbreviations

CB1=cannabinoid receptor type 1, CB2=cannabinoid receptor type 2, CBC=cannabichromene, CBCA=cannabichromenic acid, CBD=cannabidiol, CBDA=cannabidiolic acid, CBG=cannabigerol, CBGA=cannabigerolic acid, CBN=cannabinol, CBNA=cannabinolic acid, CCS=croscarmellose sodium, CMC=carboxymethyl cellulose, DW=deionized water, HPC=hydroxypropyl cellulose, NF=National Formulary, PBS=phosphate buffered saline, RDT=rapidly disintegrating tablet, SFE=supercritical fluid extraction, SSG=sodium starch glycolate, MCC=microcrystalline cellulose, THC=tetrahydrocannabinol, THCA=tetrahydrocannabinolic acid, THCV=tetrahydrocannabivarin, THCVA=tetrahydrocannabivarinic acid, USP=United States Pharmacopeia.

### I. Decarboxylated Cannabis Resin

Cannabis extracts or cannabis resins are derived from the flowers or buds harvested from Cannabis spp. plants or other parts of this plant where cannabinoids are present. Typically cannabis buds or other plant parts are subjected to extraction using various liquid solvents such as ethanol, edible oils among others, or supercritical fluids such as liquid CO₂, or gases such as butane. When such extraction is carried out, phytocannabinoids and other chemicals from the plant dissolve in these solvents, and upon concentration, the resulting material gives rise to cannabis extract or cannabis resin. Often, the chemicals in the extract or resin depend on the type of solvent utilized, temperature, pressure, extraction time etc.

The rapid disintegrating tablets of the present invention are made with decarboxylated cannabis resin derived from Cannabis spp. plants, which comprises cannabinoids which are predominately (>50%) decarboxylated. Decarboxylation refers to the conversion of the acid form to the neutral form, whereby a carboxyl group is removed from the cannabinoid molecule, and carbon dioxide is released. For example, Δ⁹-tetrahydrocannabinolic acid (Δ⁹-THCA), cannabidiolic acid (CBDA), cannabigerolic acid (CBGA), cannabichromenic acid (CBCA) and tetrahydrocannabivarinic acid (THCVA) may be decarboxylated to yield Δ⁹-tetrahydrocannabinol (Δ⁹-THC), cannabidiol (CBD), cannabigerol (CBG), cannabichromene (CBC) and tetrahydrocannabivarin (THCV), respectively. In certain embodiments, the cannabinoids in the cannabis resin are at least 55%, 60%, 65%, 70%, 75%, 80%, 85%, 90%, 91%, 92%, 93%, 94%, 95%, 96%, 97%, 98%, 99% or 100% decarboxylated, (or any integer or fractions in these ranges, for example, 96.33%) decarboxylated. As used herein, the term decarboxylated cannabis resin excludes predominately non-decarboxylated resinous substances, such as kief, hash, and hashish, which are typically heated (e.g. through smoking, vaping, or cooking) in order to achieve partial decarboxylation. In some embodiments, THC and/or CBD and/or THCV and/or CBG and/or other major cannabinoids are the major components of the decarboxylated resin, and this is dependent on the particular strain of Cannabis used in the extraction process. In addition to the decarboxylated cannabinoids, other chemicals found in Cannabis spp. and soluble in the solvent used, may be found in the resin. Such compounds may include, for example, terpenes, fatty acids, chlorophyll, flavonoids, and other compounds. Some of the compounds may undergo chemical transformation due to the processes used for extraction and carboxylation.

### Exemplary Methods for Preparing Decarboxylated Cannabis Resin

In certain embodiments, the decarboxylated cannabis resin which is used to make the rapid disintegrating tablets of the present invention may be made in accordance with the methods disclosed in U.S. Patent Application No. 62/610,706 and PCT Application No. PCT/CA2017/050788, which are herein incorporated by reference. It will be appreciated that the following methods of preparing decarboxylated cannabis resin are illustrative only and are not intended to be limiting. In alternative embodiments, other suitable methods of preparing decarboxylated cannabis resin may be used.

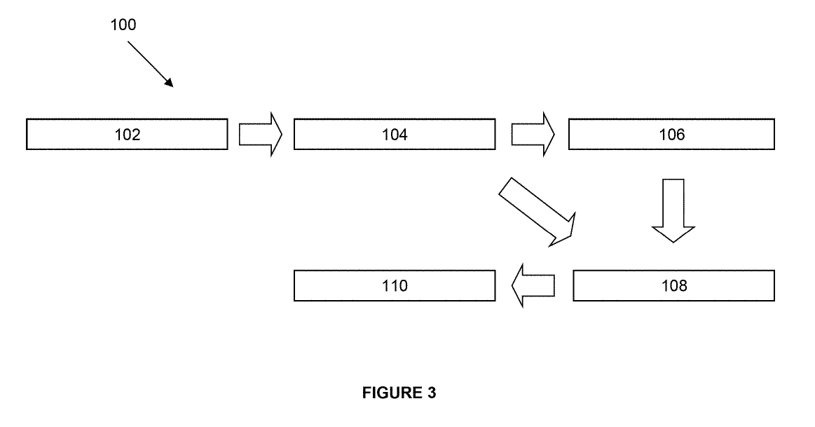

In FIG. 3, a method 100 for extracting and decarboxylating cannabinoids from cannabis plant material is shown. Generally, method 100 comprises drying raw cannabis plant material at step 102, breaking down the cannabis plant material to form cannabis plant material of a size and form suitable for extraction at step 104, extracting cannabinoids from the broken down cannabis plant material by contacting the broken down cannabis plant material with a solvent to extract the cannabinoids from the cannabis plant material and decarboxylating the cannabinoids by subjecting the extract to microwaves to form decarboxylated cannabinoids. Decarboxylation of the cannabinoids may occur when the cannabinoids are in the plant, when the cannabinoids are in the extract, or both. In some embodiments the extract is subjected to microwaves in a closed reaction vessel at a temperature and time sufficient to form decarboxylated cannabinoid.

The extracted and decarboxylated cannabinoids are optionally recovered. Recovery can include filtering the solvent from the extract of cannabis plant material to isolate the decarboxylated cannabinoids.

In some embodiments, the process comprises an extraction step before, during or after decarboxylation. In some embodiments, the process of the disclosure comprises more than one extraction step.

In some embodiments, the decarboxylation step can occur before, during or after extraction.

In an embodiment, a one-step method for extraction and decarboxylation is used, wherein the cannabis plant material (that is suitably prepared, e.g. optionally dried and broken down as described herein) is placed in a suitable extracting solvent (e.g. a pharmaceutically acceptable solvent such as ethanol, glycerol, and isopropanol, and other solvents as is known to those skilled in the art, kept in suspension or solution (e.g. by stirring, agitation, shaking or other means known to those skilled in the art) and subjected to microwave radiation while stirring at a temperature, pressure and time to obtain suitably extracted and decarboxylated cannabinoids that can be recovered for use as either a mixture or individual chemical components (e.g. for use as a therapeutic or pharmaceutical product). Further in some embodiments, the solvent can be food grade oil and/or a medium chain triglyceride, for example, coconut oil.

Such a method is more efficient in converting the cannabinoid acid into its decarboxylated form than other known methods of extraction and decarboxylation (such as simple heating).

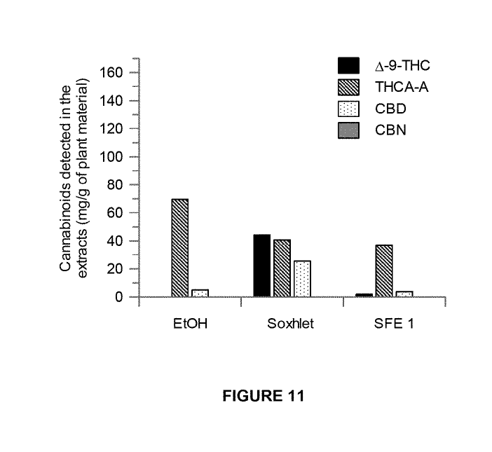

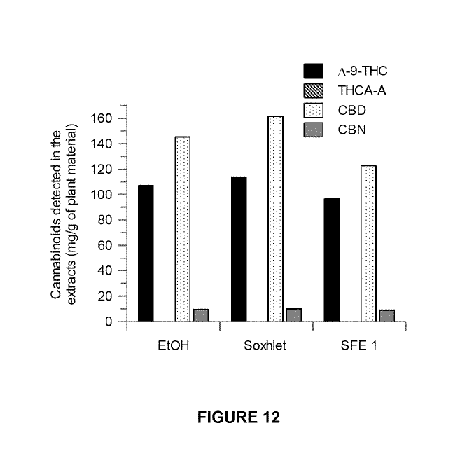

Particularly, FIGS. 11 and 12 show various concentrations of different cannabinoids in cannabis plant material extracts using ethanol, Soxhlet and SFE, without subjecting cannabis to microwave conditions and followed by microwave heating, respectively. By comparing concentrations measured through the use of solvent extraction alone, Soxhlet extraction alone (e.g. solvent and heat) and SFE alone (e.g. solvent and heat), as shown in FIG. 11, with concentrations measured by using solvent extraction followed by microwave, Soxhlet extraction followed by microwave and SFE followed by microwave, as shown in FIG. 12, adding microwave heating to each of solvent extraction, Soxhlet extraction and SFE can greatly increase THC, CBD and CBN concentrations and decrease THCA concentration in the extract.

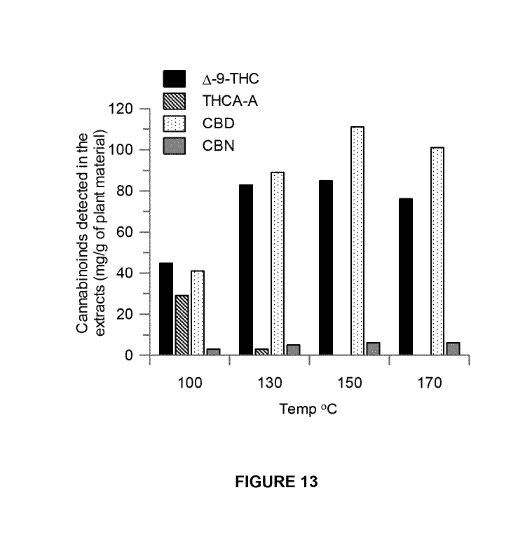

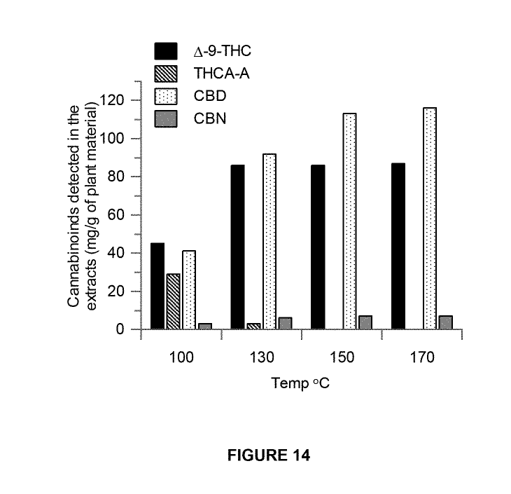

FIGS. 13 and 14 further show that high concentrations of THC, CBD, CBN and THCA in the extract are achieved when the extract is exposed to microwave radiation at about 170° C. for about 20 minutes. Further details regarding working variables are provided below.

### Drying 102

Optional drying step 102 can be used to remove excess moisture from the cannabis plant material prior to the cannabis plant material undergoing extraction and decarboxylation. Removing water content from the cannabis plant material can help to provide even heating at later stages in the extraction and/or decarboxylation process. Alternatively, fresh cannabis plant material (e.g. from the plant directly) can be used for the subsequent break down, extraction and decarboxylation steps. Herein, drying of the cannabis plant material at step 102 can occur by any means, for example in an oven at temperatures in the range of 60-75° C., or similar conditions, or using a vacuum oven or similar conditions over several hours, for example 4 hours, or 6 hours or 8 hours, depending on the amount of moisture. During the drying process, when heating is employed using heating elements in the ovens or infrared heating among other processes, some of the cannabinoid carboxyl acids forms could be converted into their decarboxylated cannabinoid forms.

### Break Down 104

At break down step 104, cannabis plant material can be broken down to produce a cannabis plant material of a size and form suitable for extraction and decarboxylation by subjecting to microwave heating.

Trichomes (i.e. resin glands) of the cannabis plant material are nearly microscopic, mushroom-like protrusions from the surface of the buds, fan leaves, and the stalk. While relatively complex, trichomes are comprised primarily of a stalk and a head. The production of cannabinoids such as THC occurs predominantly in the head of the trichome. Cannabinoids are concentrated in the trichomes of the plant. The trichome is built to easily shed from the cannabis plant material surface.

The term “a size and form suitable for extraction and decarboxylation” refers to a reduction in the particle size of the cannabis plant material fragments.

Herein, breakdown of the cannabis plant material at step 104 can occur by any mechanical means including by crushing, smashing, grinding, pulverizing, macerating, disintegration or equivalent processes as are known to those skilled in the art that reduce the cannabis plant material into small pieces suitable in size and form for extraction and/or decarboxylation.

In one example embodiment, sonication can also be used to loosen the cannabis plant material in contact with an appropriate solvent such as ethanol, and/or by breaking down cellular membranes making it suitable for extraction and/or decarboxylation. In another example embodiment, maceration can be performed with a mortar and pestle to produce a cannabis plant material of a size and form suitable for extraction and/or decarboxylation.

In certain embodiments, the cannabis plant material is reduced in size such that its particle size is within a range of 1 mm to 10 mm.

### Extraction 106

Upon completion of break down step 104, extraction step 106 may be performed. It should be noted that extraction step 106 may occur as a separate step to decarboxylation step 108 either before or after decarboxylation step 108, or as will be described below, extraction step 106 and decarboxylation step 108 may occur concurrently. For the avoidance of doubt it should be understood that any extraction, including but not limited to sonication in the presence of a solvent, reflux (Soxhlet) extraction and supercritical fluid extraction (SFE) may occur before or after decarboxylation step 108. In addition, it should also be understood that break down (104), extraction (106) and decarboxylation (108) may also occur in one step.

In one embodiment, extraction step 106 can comprise contacting cannabinoids from the broken down cannabis material that is the product of break down step 104 with a solvent.

In some embodiments, the solvent treatment in extraction step 106 removes non-cannabinoid impurities to leave a substantially pure preparation of cannabinoids. It has been shown that non-polar, liquid solvents may be useful for this function. Suitable non-polar solvents therefore include essentially any non-polar solvents which are substantially less polar than the cannabinoids, such that impurities which are more polar than the cannabinoids are removed by treatment with the solvent. Filtration and other methods as is known to those skilled in the art can also be used to remove impurities.

Useful non-polar solvents include, but are not limited to, C5-C12 straight chain or branched chain alkanes, or carbonate esters of C1-C12 alcohols. The more volatile C5-C12 alkanes may be particularly useful, as they are more easily removed from the extract. Further, solvents that have been approved for use in pharmaceutical compositions, such as ethanol (e.g. 95% ethanol) may be particularly useful.

Particularly useful solvents include pentane, hexane, heptane, iso-octane and ethanol, and/or mixtures thereof or the like as is known to those skilled in the art.

In one embodiment of extraction step 106, broken down cannabis plant material can be added to a solvent and concurrently sonicated.

Herein, sonication refers to the application of ultrasonic vibration (e.g. >20 kHz) to fragment cells, macromolecules and membranes of the dried or undried cannabis plant material. Ultrasonic vibration can be provided by any means known in the art.

In one exemplary embodiment, sonication of a mixture of cannabis plant material and solvent can occur for 5-25 minutes at 25° C., where the ratio of cannabis plant material and solvent is such that all cannabis plant material is submerged in the solvent completely in the reaction vessel.

Upon the completion of sonication of the mixture of cannabis plant material and solvent, the solvent is removed from the mixture. Removal of the solvent can occur by any means known in the art, including but not limited to filtration and/or evaporation. One embodiment for filtering after sonication is vacuum filtering over a glass sintered funnel to separate the resultant extract in the filtrate and the plant material. The latter can then be subjected to further extractions such as Soxhlet or other solvent extractions as is known to those skilled in the art, for example, SFE.

In yet another embodiment of extraction step 106, cannabinoids can be extracted from cannabis plant material that is broken down in step 104 by reflux (Soxhlet) extraction.

During reflux (Soxhlet) extraction, cannabis plant material that is broken down in step 104 is generally suspended above a heated solvent in a receptacle. The solvent is heated to reflux in a distillation flask such that solvent vapor travels up a distillation arm and floods into the receptacle housing raw cannabis material. A condenser suspended above the raw cannabis material ensures that any solvent vapor rising above the raw cannabis material cools and subsequently drips back down into the receptacle housing the raw cannabis material. The receptacle slowly fills with warm solvent such that cannabinoids begin to dissolve into the warm solvent. When the receptacle fills, it is emptied by a siphon such that the solvent is returned to the distillation flask. This cycle may be allowed to repeat many times, over hours or days.

Preferably, reflux (Soxhlet) extraction occurs at a solvent temperature higher than the boiling point of the corresponding solvent used for extraction and is conducted over a period of approximately 3 to 5 hours.

Once extraction is complete, removal of the solvent can occur by any means known in the art, including but not limited to filtering and/or evaporation as previously described.

In place of either sonication or reflux (Soxhlet) extraction as described above, another embodiment of extraction step 106 encompassed by the subject application is the extraction of cannabinoids from cannabis plant material by SFE.

SFE refers to a process of separating one or more components (extractant) from another (matrix) using supercritical fluids as the extracting solvent. Extraction is usually from a solid matrix (e.g. cannabis plant material), but can also be from liquids or resinous material (for example, hash oil).

Although numerous supercritical fluids can be used, carbon dioxide (CO₂) is the most commonly used supercritical fluid for SFE. In other exemplary embodiments, CO₂ can be modified by co-solvents such as ethanol or methanol as is known to those skilled in the art.

Extraction conditions for supercritical fluids are above the critical temperature (for example, 31° C. for CO₂) and critical pressure (for example, 74 bar for CO₂). Addition of modifiers such as but not limited to ethanol can require altering these extraction conditions.

An exemplary SFE system contains a pump for CO₂ (as well as any other solvents), a pressure cell to contain the cannabis material, a means of maintaining pressure in the system and a collecting vessel. The liquid is pumped to a heating zone, where it is heated to supercritical conditions. It then passes into the extraction vessel, where it rapidly diffuses into the solid matrix and dissolves the cannabis material to be extracted. The dissolved material (for example, cannabinoids) is swept from the extraction cell into a separator at lower pressure, and the extracted material settles out. The CO₂ can then be cooled, re-compressed and recycled, or discharged to atmosphere.

Herein, the temperature of the SFE extraction performed at extraction step 106 can, in some embodiments, be in the range of 35-55° C.

Further the pressure the SFE extraction performed at extraction step 106 can in some embodiments be in the range of 65-85 bar (6.5-8.5 MPa).

SFE in the present disclosure occurs at about 40° C. with a back pressure regulator pressure of 120 bar (12 MPa) and the extracted compounds are monitored using a photodiode array of 200-600 nm (monitoring at 254 nm). The acquisition time and method times of the system can each vary by a few minutes up to 60 minutes, ideally between 15 and 30 minutes, depending on the ratio of supercritical fluid and the co-solvent is altered for the extraction.

In specific embodiments, SFE can be carried out multiple times in succession. In such embodiments, the SFE is a fractional SFE.

As previously described for sonication with a solvent and reflux (Soxhlet) extraction, once SFE is complete, removal of the solvent can occur by any means known in the art, including but not limited to filtering and/or evaporation.

### Microwave-Assisted Extraction and Decarboxylation 108

Decarboxylation of phytocannabinoid acids such as Δ⁹-THCA is a function of the time and temperature of the reaction. For instance, the decarboxylation of concentrated Δ⁹-THCA in solution into Δ⁹-THC and the degradation of Δ⁹-THC vary with temperature. Temperature controls are therefore important for controlling desired ratios of decarboxylation products. The use of conventional household microwaves in the processing of cannabis has been discussed in the literature, however, with mixed, inconsistent results and not necessarily specifically for extraction in a solvent and decarboxylation. Further, in order to obtain 100% decarboxylation, the temperature must be sustained over a period of time without burning of the cannabis material or boiling/evaporation of the solvent. If the temperature is higher than the boiling point of the solvent employed, the solvent will boil over and/or evaporate. In order to sustain the temperature over the required period of time to fully decarboxylate the cannabinoids but not burn the cannabis plant material or boil/evaporate the solvent with the cannabis, the microwave vessel (i.e. the sealed container) must be under pressure. Sealing the vessel or container ensures pressure in the vessel or container.

As shown in FIG. 3, microwave-assisted extraction can occur either immediately after mechanical breakdown step 104 or after a preliminary extraction step 106 as described above.

Microwave assisted extraction and decarboxylation 108 can comprise suspending cannabis plant material in a solvent and subjecting the mixture to microwaves in a closed container at a temperature, pressure and time sufficient to form decarboxylated cannabinoids.

Herein, the term “microwaves” refer to a form of electromagnetic radiation with wavelengths ranging from one meter to one millimeter; with frequencies between 300 MHz (100 cm) and 300 GHz (0.1 cm).

Further, solvent treatment in microwave assisted extraction and decarboxylation step 108 is again to remove non-cannabinoid impurities to leave a substantially pure preparation of cannabinoids. As such, non-polar, liquid solvents are useful for this function. In one embodiment, ethanol is used as the liquid solvent in microwave assisted extraction and decarboxylation step 108. In another embodiment, 95% ethanol is used as the liquid solvent in microwave assisted extraction and decarboxylation step 108.

As ethanol has a boiling point of 78° C. and the decarboxylation process of cannabis is temperature dependent (as described above), temperature control is important in microwave-assisted extraction step 108.

In one exemplary embodiment, suitable conditions to promote decarboxylation of CBDA and THCA to CBD and THC, respectively, are to suspend the cannabis plant material in a solvent (such as ethanol) and then subject this mixture to electromagnetic radiation (for example, microwaves) of a wavelength in the range of 10⁶-10⁹ nm, and a frequency of 300 MHz-300 GHz. In one embodiment, the conditions further include, for example, the following: temperature range of 40-250° C., temperature increase of 2-5° C./sec, pressure range of 0-20 bar (2 MPa, 290 psi), appropriate microwave power at 2.45 GHz or minor variations and adjustments to suit a particular solvent and/or reaction conditions to reach the required temperature and accomplish decarboxylation. If stirring is required, a variable magnetic stirrer (300-900 RPM) may be used.

In another exemplary embodiment, microwave assisted extraction and decarboxylation can be performed at a temperature in the range of 100-200° C. (including all possible integers and fractions of integers in this range, for example, 163.5° C.), 130-170° C., 150-170° C. or 130-150° C.

In another exemplary embodiment, microwave assisted extraction and decarboxylation can be performed at a pressure in the range of 2-22 bar (including all possible integers and fractions of integers in this range), for example, 10.7 bar or 17 bar or 18 bar or 19 bar or 20 bar or 21 bar.

The cannabis plant material can be suspended in a solvent and subjected to microwaves at frequency and wavelength of 2.45 GHz and 1.22×10⁸ nm, respectively, and a temperature in the range of 130-190° C.

The raw cannabis material can be suspended in a solvent and subjected to microwaves at frequency and wavelength of 2.45 GHz and 1.22×10⁸ nm, respectively, and a temperature in the range of 150-190° C. and the solvent is ethanol.

In another embodiment, cannabis plant material can be suspended in a solvent and the mixture stirred for a defined period of time (e.g. 0-30 sec or a reasonable length of time so as to suspend the material) before being subjected to microwaves. In one embodiment, the defined period is 30 seconds.

In another embodiment, cannabis material can be suspended in a solvent and the mixture can be stirred while being subjected to microwaves. In one embodiment, the defined period is 10 minutes and in another embodiment the defined period is 20 minutes. A table of working microwave variables is provided below for reference.

TABLE 1

| | Total | Pre- | | Reaction |
|---|---|---|---|---|
| Temperature | time | stirring | Stir rate | Pressure |
| (° C.) | (mins) | (sec) | (rpm) | (Bar) |
| 150 | 20 | 30 | 900 | 12 |
| 150 | 20 | 30 | 900 | 11 |
| 150 | 20 | 30 | 900 | 10 |
| 150 | 20 | 30 | 900 | 13 |
| 150 | 20 | 30 | 900 | 13 |
| 150 | 20 | 30 | 900 | 12 |
| 170 | 15 | 30 | 900 | 18 |
| 150 | 10 | 30 | 600 | 8.5 |
| 150 | 10 | 30 | 600 | 8.5 |
| 150 | 10 | 30 | 600 | 9 |
| 150 | 10 | 0 | 600 | 8 |
| 150 | 10 | 0 | 600 | 8.5 |
| 150 | 10 | 0 | 600 | 8.5 |
| 100 | 30 | 0 | 600 | 1 |
| 100 | 30 | 0 | 600 | 2 |
| 100 | 30 | 0 | 600 | 1 |
| 150 | 10 | 30 | 600 | 8 |
| 150 | 10 | 30 | 600 | 9 |
| 150 | 10 | 30 | 600 | 8.5 |
| 170 | 10 | 30 | 600 | 17 |
| 150 | 10 | 30 | 600 | 10 |
| 130 | 10 | 30 | 600 | 5 |
| 100 | 5 | 30 | 600 | 2 |
| 100 | 5 | 30 | 600 | 2 |

The parameters below can be set by the user, depending on the type of microwave equipment employed and the options available for user settings:
Power (OFF)=Constant power or the maximum power applied when heating the reaction mixture.
Initial Power (OFF)=Power applied initially when heating the reaction mixture.
Fixed Hold Time (ON)=If ON, the time countdown starts when the target temperature or target pressure is reached, i.e. the initial time taken to reach the set temperature or pressure is not included in the heating time.
Pressure (OFF)=Target pressure for reaction.
Cooling (OFF)=If OFF, cooling is not applied during the heating process.
Absorption (NORMAL)=If NORMAL, power applied is initially between 200 and 400 W, depending on the target temperature.
Temperature=Target temperature. (Note: The temperature is monitored by an external infrared sensor that measures the surface temperature of the glass vial in real time.)
Total time=Total time for all steps.
Pre-stirring=Stirring time before heating process.
Stir-rate=Rotational speed of magnetic stir bar.

The parameters below were observed and extrapolated.
Power supplied=Power used to achieve the target temperature.
Holding power=Power used to maintain the target temperature.
Reaction pressure=Maximum pressure during the reaction.

In some embodiments, the decarboxylated cannabinoid product can be used directly or further processed, purified or recovered prior to use.

### Recovery 110

Optionally, after being subjected to microwaves, a preparation of decarboxylated cannabinoids can be recovered from the resulting suspension at recovery step 110.

In one embodiment, extracted and decarboxylated cannabinoids are recovered by filtering the solvent from the extract of cannabis plant material to isolate the decarboxylated cannabinoids or decarboxylated cannabinoid comprising fraction.

In another embodiment, extracted and decarboxylated cannabinoids are recovered by filtering through an appropriate Celite® pad and/or activated carbon (e.g. charcoal) to obtain clarified solution for subsequent processing or use. In this embodiment, Celite® can be placed in a glass sintered funnel and then layered with activated carbon. Filtering agents can be washed with ethanol via vacuum filtration and extract can be dissolved in appropriate volume of suitable solvent such as ethanol and transferred to a funnel. Vacuum can then be applied and the filtering agent can be washed with the solvent until cannabinoids are completely eluted. The resulting filtrate can then be concentrated to dryness (e.g. at 25° C.). Someone with skill in the art can also conceive employing functionalized membranes, cellulose filters or the like to accomplish the above recovery task, instead of Celite® and activated carbon pad.

Alternatively, the resulting preparation of decarboxylated cannabinoids from step 108 can be collected and subsequently processed according to any of the extraction methods described in step 106, including but not limited to sonication, reflux (Soxhlet) extraction and/or SFE.

### EXAMPLES

The following examples from U.S. Patent Application No. 62/610,706 and PCT Application No. PCT/CA2017/050788, which are herein incorporated by reference, are illustrative of methods of obtaining decarboxylated cannabis resin which may be used for making the rapid disintegrating tablets of the present disclosure. It will be appreciated that these examples are illustrative only and other suitable methods may be used.

### Experiment 1: Decarboxylated cannabis Resin

Medicinal cannabis was subjected to extraction and chemical analysis by UPLC-MS.

### Extraction Methodologies

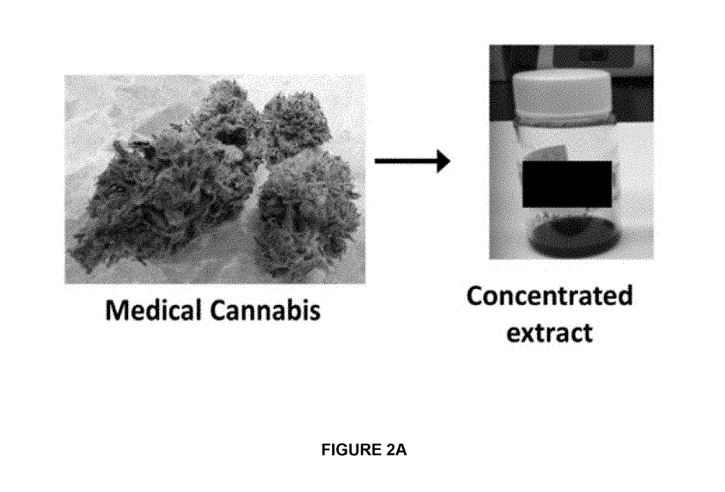

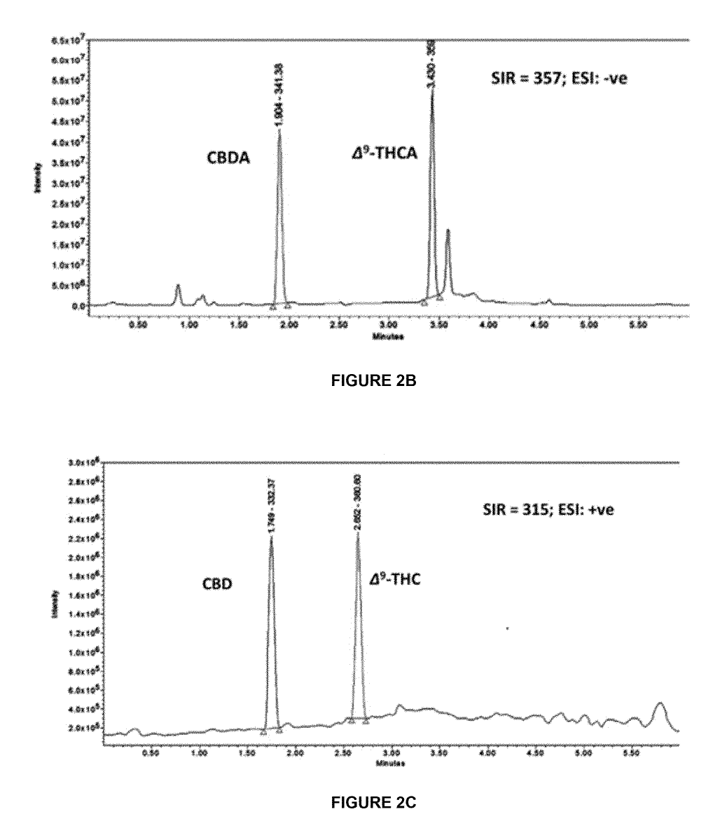

FIG. 2A shows medical cannabis and decarboxylated cannabis resin. Four types of extraction methods were performed as shown in the corresponding flow charts in Scheme 1. (A) Ultrasonication (B) Sohxlet extraction (C) Supercritical Fluid Extraction (Omar, J. Olivares, M. et al. J. Sep. Sci. 2013, 36, 1397-1404, incorporated herein by reference) (D) Closed system microwave extraction, as described in experiments 5-9 below.

Scheme 1. Flow Charts of Various Extractions Methods

UPLC-MS Methodology. Cannabinoid standards, cannabis extracts and cannabinoids in the donor samples were analyzed using Waters® ACQUITY UPLC H-Class System equipped with Quaternary Solvent Manager and Sample Manager FTN. The detector used to monitor the samples was Waters® MS 3100 mass spectrometer. Benzophenone, caffeine or Δ⁹-THC-d₃ was used as an internal standard. Conditions are listed in Table 2.

TABLE 2
UPLC-MS Chromatographic conditions
UPLC-MS Conditions

Column: BEH (2.1 × 50 mm, C₁₈, 1.7 μm)
Mass scan range (ESI +ve and −ve): 150-500 m/z
Flow rate: 0.6 mL/min
Solvent: H₂O (0.1% formic acid; solvent A)
MeOH (0.1% formic acid; solvent B)
Gradient conditions:
0-4.5 min: 30% A/70% B-100% B
4.5-5.0 min: 100% B
5.0-5.2 min: 100% B-30% A/70% B
5.2-6.0 min: 30% A/70% B

### Extraction Results

TABLE 3
Quantities of extracts and major cannabinoids obtained
from extraction Methods A-D of Strain I.

| | | | Cannabinoid in resin (%) | |
|---|---|---|---|---|
| | Plant | Extract | Total Δ⁹-THC | Total CBD |
| Method | used (g) | isolated (g) | (THCA + THC) | (CBDA + CBD) |
| A | 1.51 | 0.453 | 31.2 ± 0.5 | 66.1 ± 1.4 |
| B | 2.00 | 0.616 | 16.7 ± 0.2 | 33.3 ± 0.6 |
| C | 1.00 | 0.277 | 30.4 ± 0.9 | 59.3 ± 1.9 |
| D | 1.01 | 0.211 | 29.4 ± 7.6 | 33.8 ± 8.6 |

TABLE 4
Quantities of extracts and major cannabinoids obtained
after Method D (closed system microwave extraction).

| | Plant | Extract | Cannabinoid in resin (%) | |
|---|---|---|---|---|
| Strain | used (g) | isolated (g) | Δ⁹-THC | CBD |
| I | 1.01 ± 0.0003 | 0.211 ± 0.01 | 29.4 ± 7.6 | 33.8 ± 8.6 |
| II | 1.00 ± 0.0004 | 0.154 ± 0.004 | 1.9 ± 0.2 | 37.6 ± 4.1 |
| III | 1.00 ± 0.002 | 0.210 ± 0.009 | 49.8 ± 0.9 | 0 |

### Chemical Analyses by UPLC-MS

Mass spectra of Strain 1 before (FIG. 2b) and after (FIG. 2c) decarboxylation of medical cannabis, indicates 100% conversion of CBDA and THCA into CBD and THC, respectively.

Closed system microwave extraction provides simultaneously extraction and decarboxylation of the cannabinoids, as observed during chemical analysis by UPLC-MS.

### Experiment 2: Further Comparison of Various Extraction Methods, Decarboxylation of Cannabinoids and Production of Standard Curves

Method 1A: Ultrasonic Extraction (Sonication).

General Procedure:
1. Dried plant material was weighed and macerated using a mortar and pestle.
2. Solvent was added (25-130 mL) and mixture sonicated for 5 mins at 25° C.
3. Solvent was decanted and filtered over a glass sintered funnel using a vacuum filtration.
4. Steps 2 and 3 were repeated twice with the remaining fibre material.
5. Filtrate was concentrated to dryness (at 25° C.) then weighed (green resin).

Table 5 below shows the results of sonication of three common strains of cannabis.

TABLE 5
Results of sonication extraction with various
solvents on three strains of cannabis.

| | | Solvent | | |
|---|---|---|---|---|
| | | Hex- | Isopropanol/hexanes | |
| Strain | | anes | (1:1) | Ethanol |
| Strain 1 | Plant Used (g) | 0.256 | 1.0013 | 0.246 |
| (THC: 7.18/ | | | | 1.5084 |
| CBD: 8.6) | Resin | 0.0617 | 0.4748 | 0.1802 |
| | produced (g) | | | 0.4531 |
| Strain 2 | Plant Used (g) | N/A | N/A | 2.0018 |
| (THC: 0/ | Resin | N/A | N/A | 0.4795 |
| CBD: 9) | produced (g) | | | |
| Strain 3 | Plant Used (g) | | N/A | 2.9475 |
| (THC: 18.6/ | Resin | N/A | N/A | 1.1611 |
| CBD: 0) | produced (g) | | | |

Method 1B: Filtration Over Celite®/Activated Carbon.

Following Method 1A (described above), extracts were subjected to filtration over Celite® and activated carbon in order to eliminate the green colour of extracts. The results are shown in Table 6.

General Procedure:
1. Celite® was placed in a glass sintered funnel, then layered with activated carbon.
2. Extract was dissolved in 1 mL ethanol and transferred to funnel.
3. Vial that contained extract was washed twice with 1.5 mL ethanol and transferred to funnel.
4. Vacuum was then applied and filtering agent washed with ethanol until filtrate was no longer UV active (60-70 mL).
5. Filtrate was concentrated to dryness (at 25° C.), then weighed (orange resin).

TABLE 6
Results of extract filtration with Celite ®/Activated
Carbon for three strains of cannabis

| Strain | Extract used (g) | Extract isolated (g) |
|---|---|---|
| Strain 1 | 0.2265 | 0.2095 |
| (THC: 7.18/CBD: 8.6) | | |
| Strain 2 | 0.2026 | 0.1717 |
| (THC: 0/CBD: 9) | | |
| Strain 3 | 0.4984 | 0.4561 |
| (THC: 18.6/CBD: 0) | | |

Method 2: Soxhlet Extraction

General Procedure:
1. Dried plant material was weighed and macerated using a mortar and pestle
2. Crushed material was then transferred to a cellulose extraction thimble (43×123 mm; 2 mm thickness)
3. Thimble was then inserted into a large extractor (size: 55/50)
4. Solvent (400 mL) and stir bar were added to a round bottom flask, which was then placed in the suitable DrySyn heating block and connected to the extractor
5. Extractor was then connected to a large condenser (size: 55/50) and refluxing was done at 120° C. for 3.5 hrs
6. Once refluxing was complete, the solvent was concentrated to dryness (at 25° C.) then weighed (green resin)

Table 7 below provides the results of Soxhlet extraction of cannabis according to the forgoing procedure.

TABLE 7
Results of Soxhlet extraction of cannabis.

| Strain | Amount of plant used (g) | Extract isolated (g) |
|---|---|---|
| Strain 1 | 2.0021 | 0.6163 |
| (THC: 7.18/CBD: 8.6) | | |

Method 3: Supercritical Fluid Extraction (SFE)

General Procedure:
1. Dried plant material was weighed and macerated using a mortar and pestle
2. Crushed plant material was transferred to a 10 mL extraction vessel and subjected to either of the following conditions below
3. All fractions were combined and concentrated to dryness (at 25° C.) then weighed (green resin)

Method 3A: SFE Conditions

| | |
|---|---|
| Solvent A = CO₂ | Solvent B = ethanol |
| Temperature = 40° C. | BPR = 12 MPa |
| PDA = 200-600 nm (monitoring at 254 nm) | |
| Acquisition time = 30 mins | Method time = 30.2 mins |

i. 5 mins static with 1:1 A/B
ii. 25 mins dynamic with 1:1 A/B (Flow rate=10 mL/min; make up pump=0.2 mL/min)
iii. Fractions were collected every 5 mins

Method 3B: SFE Conditions

| | |
|---|---|
| Solvent A = CO₂ | Solvent B = ethanol |
| Temperature = 40° C. | BPR = 12 MPa |
| PDA = 200-600 nm (monitoring at 254 nm) | |

i. 15 mins dynamic with 100% A (Flow rate=10 mL/min)
   Fractions were collected every 7.5 mins
   Acquisition time=15 mins Method time=15.2 mins
ii. 30 mins dynamic with 80:20 A/B (Flow rate=10 mL/min; make up pump=1 mL/min)
   Fractions were collected every 5 mins
   Acquisition time=30 mins Method time=30.2 mins

Table 8 below shows the results of SFE according to the conditions outlined in Methods 3A and 3B for cannabis.

TABLE 8
Results of SFE extraction of cannabis according to two sets of
conditions.

| | | Amount | |
|---|---|---|---|
| Strain | Conditions | of plant used (g) | Extract isolated (g) |
| Strain 1 | Method 3A | 1.0047 | 0.2164 |
| (THC: 7.18/CBD: | Method 3B | 1.0043 | 0.2561 |
| 8.6) | | | |

Method 4: Microwave-Assisted Extractions with Ethanol (MAE) Followed by SFE.

Suitable conditions to promote decarboxylation of CBDA and THCA to CBD and THC, respectively, were determined with MAE.

The solvent used for this extraction was ethanol. However, since ethanol has a boiling point of 78° C., the highest temperature that could be achieved when heating only ethanol in a sealed vessel under microwave conditions had to be determined.

General Procedure:
1. Ethanol (11 mL) and a stir bar were placed in a 20 mL microwave vial which was then sealed.
2. General microwave conditions:
   (a) Pre-stirring=30 secs
   (b) Run time=15 mins
   (c) Absorption=Normal

The results are shown in Table 9 below.

TABLE 9
Determination of highest temperature that could be achieved when
heating only ethanol in a sealed vessel under microwave conditions.

| Attempt # | Temperature (° C.) | Results |
|---|---|---|
| 1 | 200 | Maximum system pressure attained; |
| | | Run aborted due to high pressure |
| 2 | 150 | Run completed |
| 3 | 190 | Maximum system pressure attained; |
| | | Run aborted due to high pressure |
| 4 | 180 | Maximum system pressure attained; |
| | | Run aborted due to high pressure |
| 5 | 170 | Run completed |

Once the maximum temperature that ethanol could be heated was determined, the following conditions were performed with cannabis:

General Procedure:
1. Dried plant material was weighed and macerated using a mortar and pestle
2. Crushed plant material was transferred to a 20 mL or 5 mL microwave vial along with a stir bar
3. Ethanol (11 mL or 3 mL) was added to the vial such that the plant material was completely submerged. The vial was then sealed and subjected to the microwave conditions below:
   (a) Pre-stirring=30 secs
   (b) Run time=10 mins
   (c) Absorption=Normal
4. The suspension was filtered and the filtrate and plant fibre collected separately
5. Filtrate was concentrated and plant fibre subjected to the SFE conditions below:

| | |
|---|---|
| Solvent A = CO₂ | Solvent B = ethanol |
| Temperature = 25° C. | BPR = 12 MPa |
| PDA = 200-600 nm (monitoring at 254 nm) | |
| Acquisition time = 20 mins | Method time = 20.2 mins |

6. Gradient from 100% A to 50%; 0.1 mins-15 mins (Flow rate=10 mL/min; make up pump=1 mL/min)
7. Fractions were collected every 7.5 mins

The results are shown in Table 10 below.

TABLE 10
Extraction of cannabis by Microwave and SFE at various
microwave temperatures

| | | | | Extract | Extract |
|---|---|---|---|---|---|
| | | | Amount of | isolated after | isolated |
| | | Temperature | plant used | microwave | after SFE |
| Strain | Method | (° C.) | (g) | (g) | (g) |
| Strain 1 | A | 100 | 1.0012 | 0.3246 | N/A |
| (THC: | B | 130 | 0.2554 | 0.0592 | 0.0008 |
| 7.18/ | C | 150 | 0.2543 | 0.063 | 0.0005 |
| CBD: 8.6) | D | 170 | 0.2554 | 0.0644 | 0.0025 |

Table 14 (provided below) shows the analyses and quantification of the cannabinoids.

Method 5. SFE/Soxhlet/Sonication Extraction Followed by Microwave of the Resin (for Decarboxylation).

General Procedure:
1. Resin isolated from Methods 1A (ethanolic extract), 2 and 3A were dissolved in 3-3.5 mL ethanol and transferred to a 5 mL microwave vial.
2. A stir bar was added and the vial sealed and subjected to microwave conditions below:
   (a) Temperature=150° C.
   (b) Pre-stirring=30 secs
   (c) Run time=10 mins
   (d) Absorption=Normal
3. The reaction mixture was then concentrated.

The results are shown in Table 11 below.

TABLE 11
Weights of resins after subjecting to microwave heating.

| | | Amount of | Resin isolated after |
|---|---|---|---|
| Strain | Method | resin (g) | microwave (g) |
| Strain 1 | 1A (Sonication) | 0.2223 | 0.1692 |
| (THC: 7.18/CBD: | 2 (Soxhlet) | 0.2651 | 0.2211 |
| 8.6) | 3A (SFE; 50:50) | 0.2164 | 0.1884 |

Chromatography Analyses (HPLC/MS/PDA)

The chromatographic profiles of the cannabis extracts were determined by LC-PDA-MS equipped with a Waters® 2545 binary gradient module LC, Waters® PDA2998 photodiode array detector (190-800 nm) and a Waters® 3100 mass spectrometer (60-2000 Da).

LC was performed on an X-Bridge analytical C18 column (4.6 mm×150 mm, 5 um I.D.) with 1.5 mL/min flow rate. Mass spectra were recorded using ESI (+ve) mode. The injection samples were filtered using Millex-GV Syringe Filters (0.22 μm, EMD Millopore).

Chromatographic conditions were as follows:

Mobile Phase:
A: Water/0.1% formic acid
B: Methanol/0.1% formic acid

Gradient:
0 to 25 min: 30% A/70% B→100% B
25 to 28 min: 100% B
28 to 30 min: 100% B→30% A/70% B
30 to 35 min: 30% A/70% B

Injection volume: 10 μL

Flow rate: 1.5 mL/min

Total run time: 35 min

UPLC/MS.

The cannabis extracts and cannabinoid standards were analyzed using Waters® ACQUITY UPLC H-Class System equipped with Quaternary Solvent Manager, Sample Manager FTN, Acquity UPLC® BEH column (2.1×50 mm, C18, 1.7 μm). The sample injection plate and the column were maintained at 15° C. and 40° C., respectively. The detector used to monitor the samples was Waters® MS 3100 mass spectrometer.

Chromatographic conditions were as follows:

Mobile Phase:
A: Water/0.1% formic acid
B: Methanol/0.1% formic acid

Gradient:
0 to 4.5 min: 30% A/70% B→100% B
4.5 to 5.0 min: 100% B
5.0 to 5.2 min: 100% B→30% A/70% B
5.2 to 6.0 min: 30% A/70% B

Injection volume: 2 μL

Flow rate: 0.6 mL/min

Total run time: 6 min

Standard Curves for Cannabinoids:

Standard cannabinoids samples were purchased from Cerilliant-Certified Reference Standards in the form of 1.0 mg/mL solution in methanol.
1. Δ⁸-Tetrahydrocannabinol (Δ⁸-THC, Cat #. T-032, Lot FE10011501)
2. Δ⁹-Tetrahydrocannabinol (Δ⁹-THC, Cat #. T-005, Lot FE05271502)
3. Δ⁹-Tetrahydrocannabinolic acid A (THCA-A, Cat #. T-093, Lot ER02101506)
4. Δ²-Cannabidoil (CBD, Cat #. C-045, Lot FE012881502)
5. Cannabidiolic acid (CBDA, Cat #. C-144. Lot FE0181602)
6. Cannabinol (CBN, Cat #. C-046, Lot FE06081502)

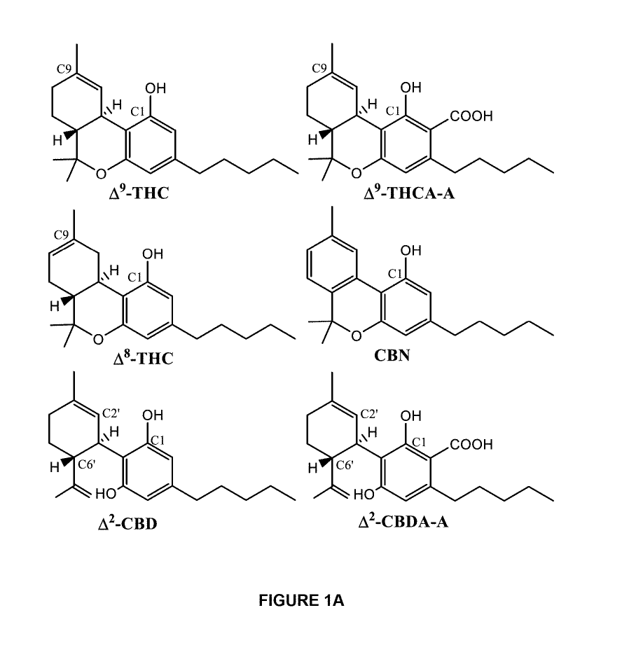

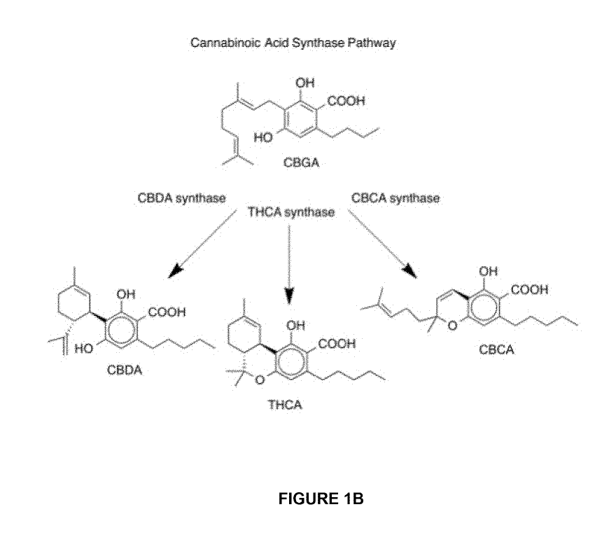

The chemical structures of cannabinoids 1-6 are provided in FIG. 1.

Working stock solution of each standard sample was prepared using water/0.1% formic acid and methanol/0.1% formic acid. The final concentration of each stock sample was 50 μg/mL in 30% water/0.1% formic acid and 70% methanol/0.1% formic acid.

The stock samples (50 μg/mL) were diluted with mobile phase (30% water/0.1% formic acid and 70% methanol/0.1% formic acid) to obtain the following concentrations: 0, 0.1, 0.5, 1.0, 2.5, 5.0, 7.5, and 10.0 μg/mL

Not all the concentrations were included in the construction of the standard curve. Some of the cannabinoids (e.g. 0.1 or 10.0 μg/mL) were excluded due to very low signal of saturation level.

Each concentration was run in triplicate. 2 μL injections were made and the signal was recorded for up to 6 minutes. SIR +ve (311, 315, and 359 m/z) or SIR −ve (313 and 357) and mass scan (150-500 m/z) in positive mode were monitored and recorded. SIR chromatograms were integrated and the AUC was plotted vs. concentration (μg/mL).

TABLE 12
Cannabinoids standard curve - summary.

| Reference compound | Retention time (min) | Range (μg/mL) |
|---|---|---|
| Δ²-CBD | 1.70 | 0.1-10.0 |
| CBD-A | 1.90 | 0.01-1.0 |
| CBN | 2.33 | 0.1-7.5 |
| Δ⁹-THC | 2.58 | 0.1-7.5 |
| Δ⁸-THC | 2.70 | 0.1-10.0 |
| THCA-A | 3.42 | 0.01-1.0 |

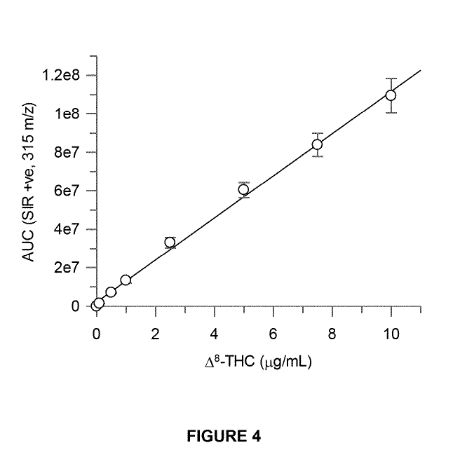

Standard curves for each of cannabinoids 1-6 as described above are provided in FIGS. 4-9.

FIG. 4 shows a Standard Curve for Δ⁸-THC. Linear fit: y=10948149x+2153365, R=0.9984

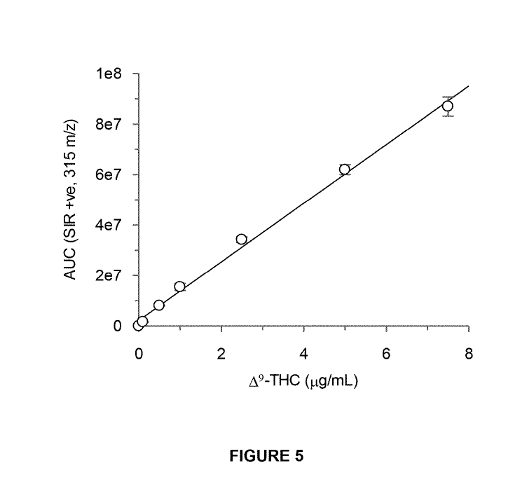

FIG. 5 shows a Standard Curve for Δ⁹-THC. Linear fit: y=11609869x+2187215, R=0.9980.

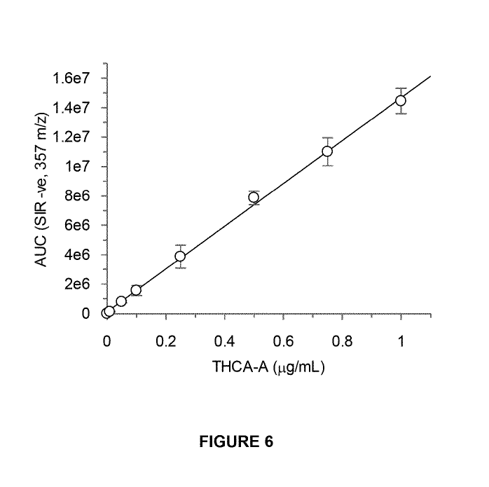

FIG. 6 shows a Standard Curve for THCA. Linear fit: y=14550967x+119886, R=0.9992

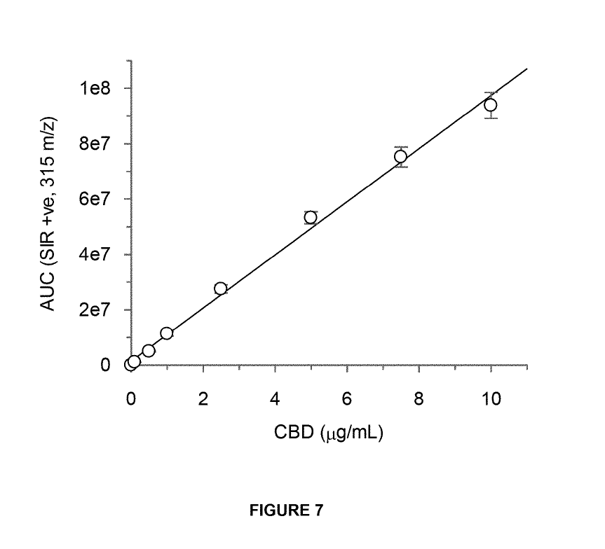

FIG. 7 shows a Standard Curve for CBD. Linear fit: y=9601901x+1448932, R=0.9978.

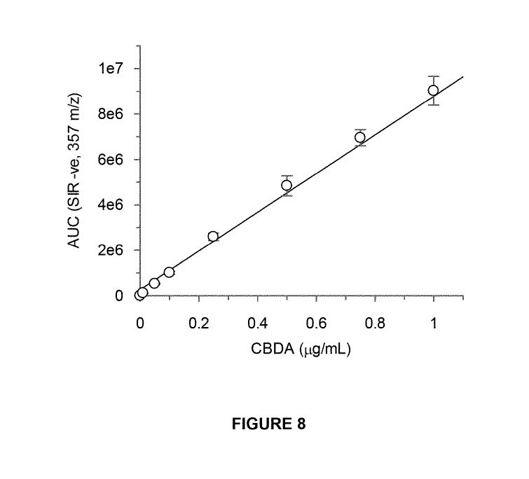

FIG. 8 shows a Standard Curve for CBDA. Linear fit: y=9096880x+111409, R=0.9993.

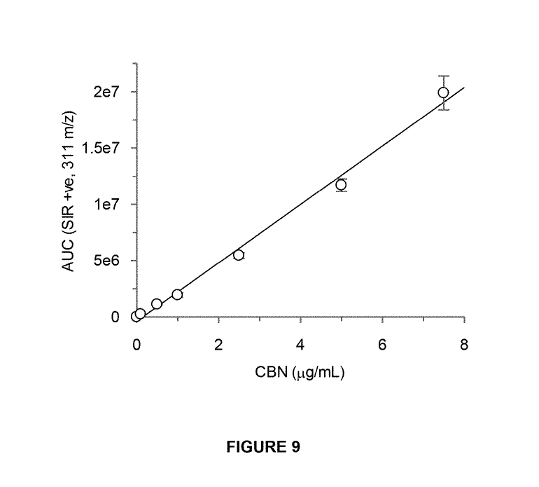

FIG. 9 shows a Standard Curve for CBN. Linear fit: y=2595328x−397594, R=0.9967.

Table 13 provides the concentration (μg/mL) of cannabinoids in extracts obtained using microwave extraction method at different temperatures. The solid material (plant fiber) leftover after the microwave reaction was exposed to SFE extraction (method 3A). The total volume of each microwave reaction was 3 mL.

TABLE 13
Concentration (μg/mL) of cannabinoids in extracts obtained using
microwave extraction method at different temperatures

| Extraction conditions | Δ⁸-THC | Δ⁹-THC | THCA | CBD | CBN |
|---|---|---|---|---|---|
| Microwave, 100° C., 10 min; | 0 | 554 | 358 | 500 | 252 |
| No SFE done for this | N/A | N/A | N/A | N/A | N/A |
| sample | | | | | |
| Microwave, 130° C., 10 min | 0 | 7046 | 225 | 7565 | 443 |
| SFE | 0 | 243 | 0 | 302 | 32 |
| Microwave, 150° C., 10 min | 0 | 7201 | 0 | 9408 | 508 |
| SFE | 0 | 86 | 0 | 135 | 82 |
| Microwave, 170° C., 10 min | 0 | 6502 | 0 | 8566 | 495 |
| SFE | 0 | 936 | 0 | 1321 | 120 |

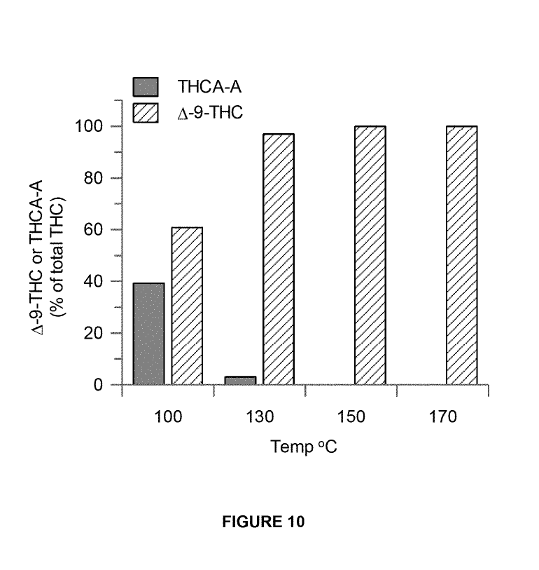

FIG. 10 shows the amount of Δ⁹-THC and THC-A present in samples extracted using microwave method at different temperatures. The amount of each form of THC is shown as a percentage of total amount of THC (neutral and acidic form).

Table 14 shows the amount of cannabinoids in the cannabis extracts, after subjecting to Method 5. See also Table 11. Note: CBN appears to be formed during the microwave based decarboxylation of THCA. CBDA was not quantified.

TABLE 14
Amount of cannabinoids in the cannabis extracts, after subjecting
to Method 5

| | Δ⁸-THC | Δ⁹-THC | Δ⁹-THCA-A | Δ²-CBD | CBN |
|---|---|---|---|---|---|
| | mg/g | mg/g | mg/g | mg/g | mg/g |
| Sonication | 0 | 0 | 69.47 | 4.93 | 0 |
| (Method 1A) | | | | | |
| Soxhlet alone | 0 | 44.32 | 40.71 | 25.4 | 0 |
| (Method 2) | | | | | |
| SFE alone | 0 | 1.91 | 36.91 | 3.68 | 0 |
| (Method 3A) | | | | | |
| Sonication + μw | 0 | 107.18 | 0 | 145.1 | 9.53 |
| (Method 1A and 5) | | | | | |
| Soxhlet + μw | 0 | 113.98 | 0 | 161.75 | 10.08 |
| (Method 2 and 5) | | | | | |
| SFE + μw | 0 | 96.48 | 0 | 122.66 | 8.96 |
| (Method 3A and 5) | | | | | |

μW = microwave
ND = not determined

A plot of the above data is shown in FIGS. 11 and 12, wherein FIG. 11 illustrates concentrations in the extracts using EtOH, Soxhlet and SFE, without subjecting cannabis to microwave conditions and FIG. 12 shows the concentrations of cannabinoids first extracting cannabis using EtOH or Soxhlet or SFE followed by microwave heating.

Table 15 shows the yield (mg/g of plant material) of cannabinoids in the extracts obtained using microwave extraction method at different temperatures. The solid material (plant fiber) left over after the microwave reaction was exposed to SFE extraction (method 3A). The total volume of each microwave reaction was 3 mL. CBDA was not quantified.

TABLE 15
Yield (mg/g of plant material) of cannabinoids in extracts obtained
using microwave extraction method at different temperatures

| | Δ⁸-THC | Δ⁹-THC | THCA | CBD | CBN |
|---|---|---|---|---|---|
| Conditions | mg/g | mg/g | mg/g | mg/g | mg/g |
| Microwave 100° C., 10 min | 0 | 45 | 29 | 41 | 3 |
| No SFE | N/A | N/A | N/A | N/A | N/A |
| Microwave, 130° C., 10 min | 0 | 83 | 3 | 89 | 5 |
| Follow-up SFE | 0 | 3 | 0 | 4 | 0 |
| Microwave, 150° C., 10 min | 0 | 85 | 0 | 111 | 6 |
| Follow-up SFE | 0 | 1 | 0 | 2 | 1 |
| Microwave, 170° C., 10 min | 0 | 76 | 0 | 101 | 6 |
| Follow-up SFE | 0 | 11 | 0 | 16 | 1 |

FIG. 13 shows cannabinoids obtained from cannabis after microwave heating only.

FIG. 14 shows microwave followed by SFE extraction. The cannabinoids from microwave solvent and SFE extractions added together.

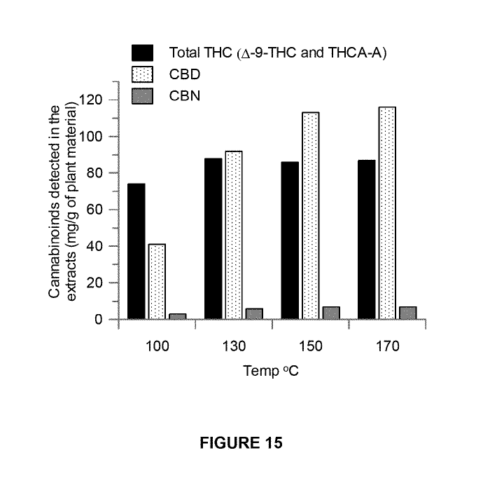

FIG. 15 shows various cannabinoid concentrations from cannabis after microwave heating followed by SFE extraction. In FIG. 15, the cannabinoids from both extractions have been added together. The acidic and neutral form of THC were added together to represent the total THC extracted by each method.

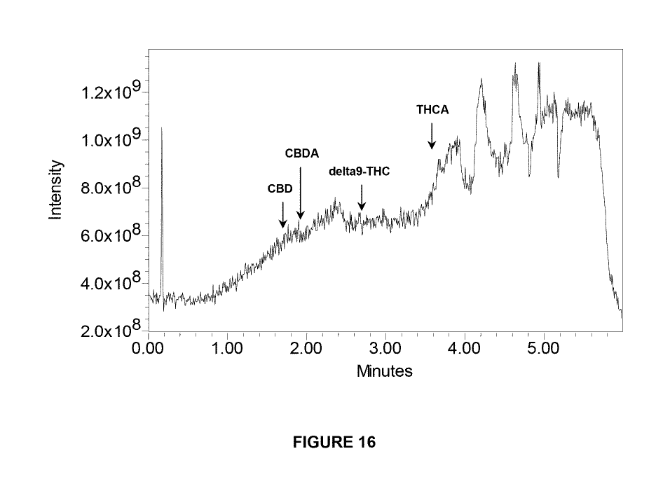

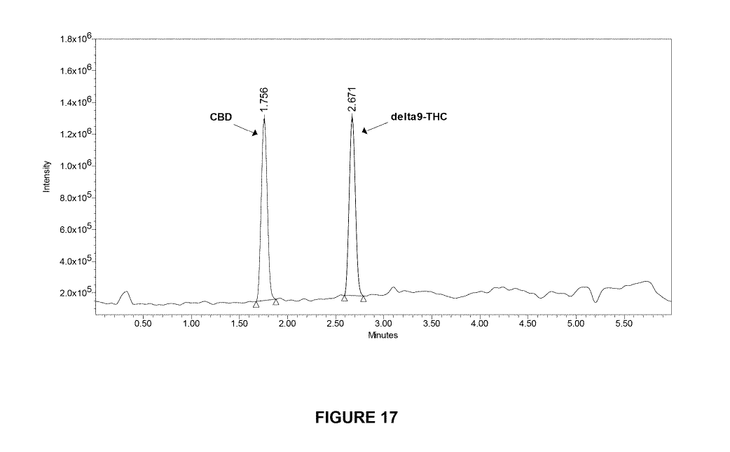

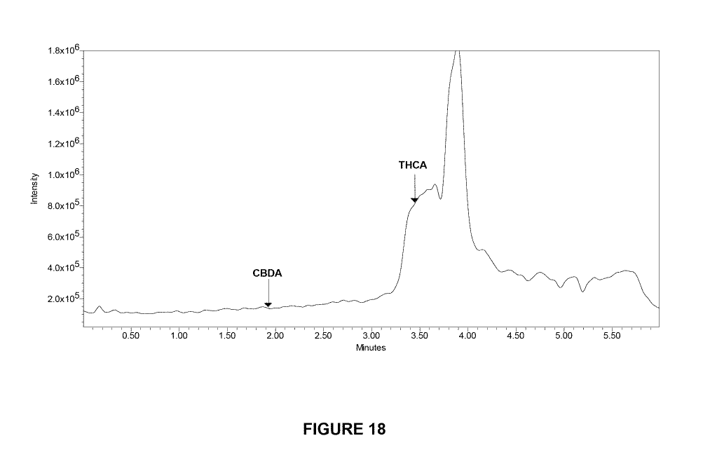

FIG. 16 shows a chromatogram of mass scan (150-500 m/z) recorded in positive mode of ESI using Waters MS3100 mass detector. The sample was obtained from microwave extraction at 170° C., 15 min. The arrows point to the retention times of CBD, CBDA, Δ⁹-THC, and THCA. Due to the low sample concentration the peaks are not visible in this chromatogram. Single ion recording (SIR +ve) obtained for this sample shows the individual peaks representing CBD and Δ⁹-THC (FIG. 17), CBDA and THCA (FIG. 18). If an analyte is absent or not detected in this mode, the expected retention time is shown.

FIG. 17 shows a Single Ion Recording (SIR +ve, m/z=315) to detect Cannabidiol (CBD) and Δ⁹-Tetrahydrocannabinol (Δ⁹-THC) in a sample obtained from microwave extraction at 170° C., 15 min (mass scan shown in FIG. 16).

FIG. 18 shows a Single Ion Recording (SIR +ve, m/z=359) to detect Cannabidiolic acid (CBDA) and Tetrahydrocannabinolic acid (THCA) in a sample obtained from microwave extraction at 170° C., 15 min (mass scan shown in FIG. 16). Positive mode (ESI +ve) is not a favorable one for the detection of the acidic forms of cannabinoids.

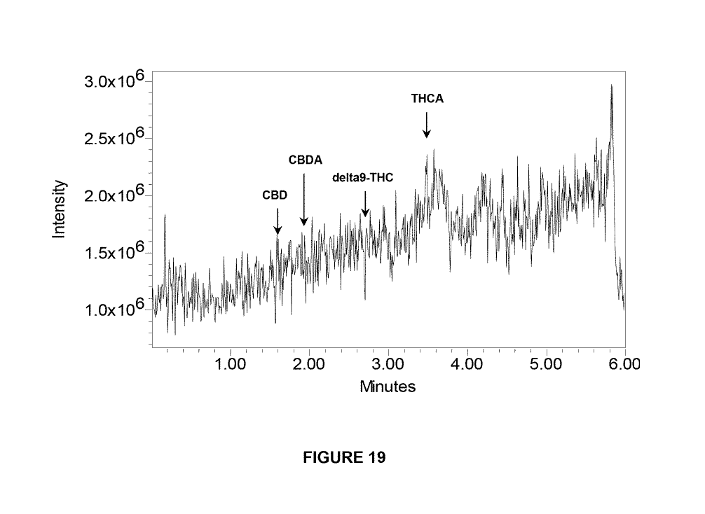

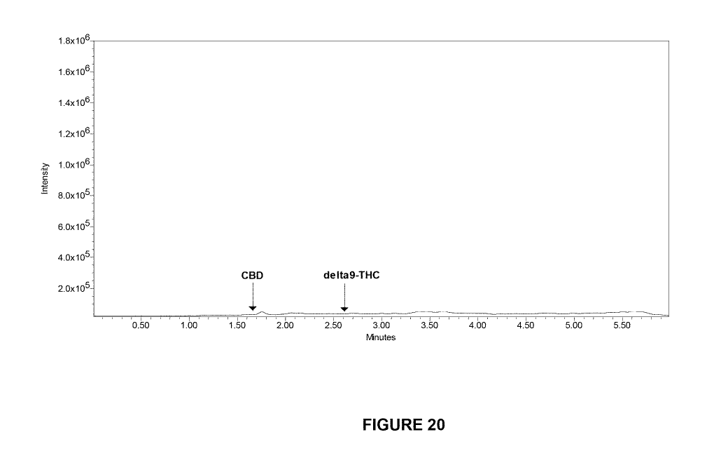

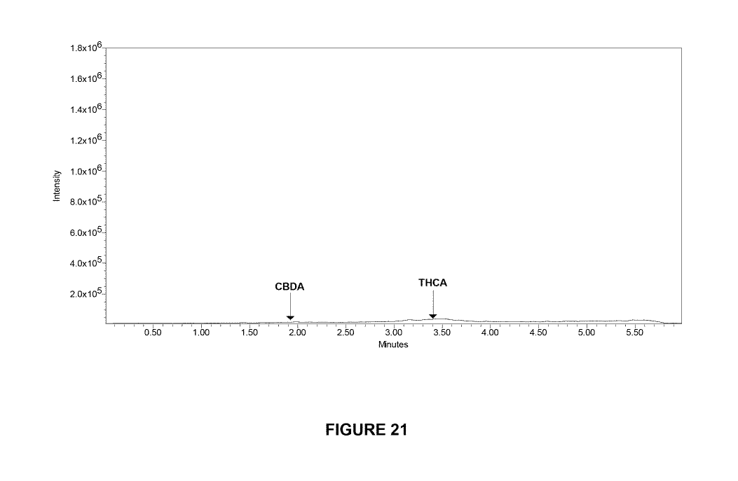

FIG. 19 shows a chromatogram of mass scan (150-500 m/z) recorded in negative mode of ESI using Waters® MS3100 mass detector. The sample was obtained from microwave extraction at 170° C., 15 min. The arrows point to the retention times of CBD, CBDA, Δ⁹-THC, and THCA. Due to the low sample concentration the peaks are not visible in this chromatogram. Single ion recording (SIR −ve) obtained for this sample shows the individual peaks representing CBD and Δ⁹-THC (FIG. 20), CBDA and THCA (FIG. 21). If an analyte is absent or not detected in this mode, the expected retention time is shown.

FIG. 20 shows a Single Ion Recording (SIR −ve, m/z=313) to detect Cannabidiol (CBD) and Δ⁹-Tetrahydrocannabinol (Δ⁹-THC) in a sample obtained from microwave extraction at 170° C., 15 min (mass scan shown in FIG. 19). The neutral forms of cannabinoids are not easily detectable in the negative mode therefore no peaks observed as compared to the positive mode (see FIG. 17).

FIG. 21 shows a Single Ion Recording (SIR −ve, m/z=357) to detect Cannabidiolic acid (CBDA) and Tetrahydrocannabinolic acid (THCA) in a sample obtained from microwave extraction at 170° C., 15 min (mass scan shown in FIG. 19). ESI −ve mode is useful for the detection of the acidic forms of cannabinoids.

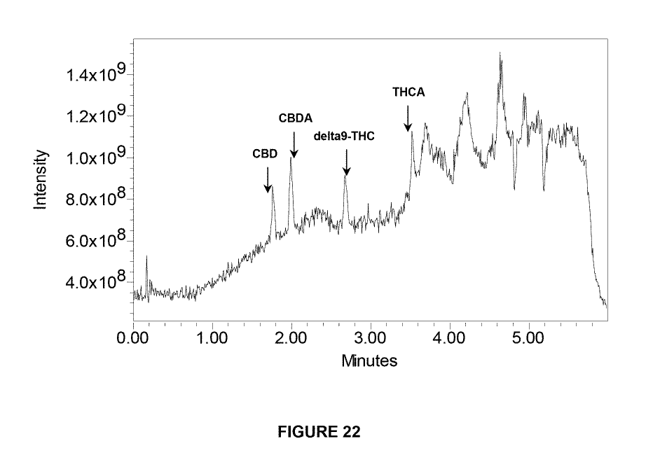

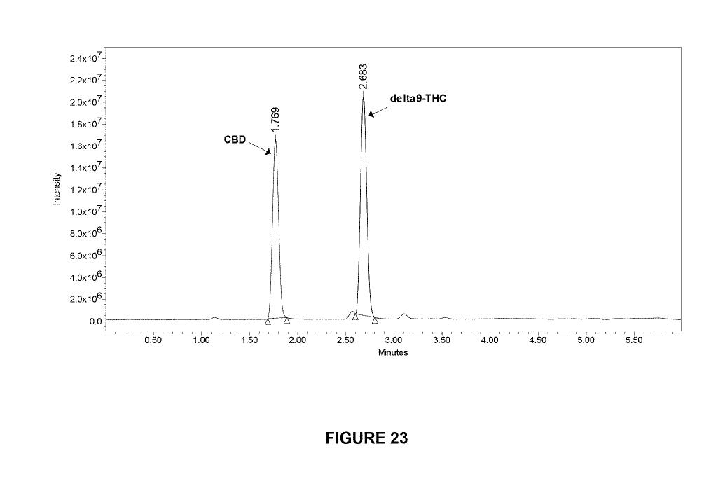

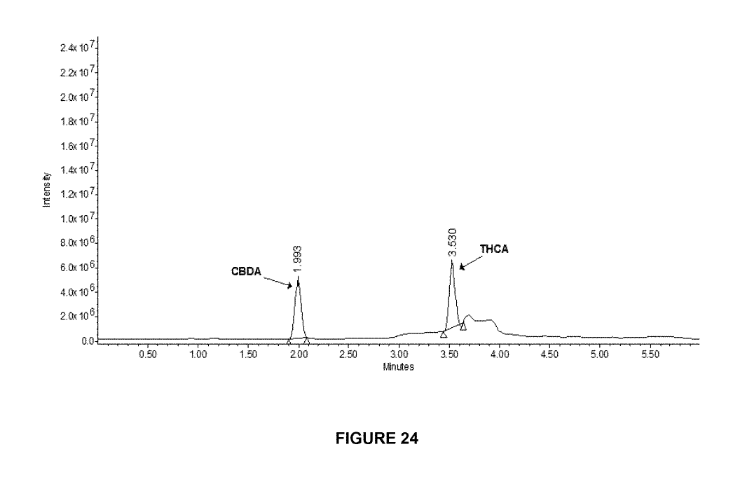

FIG. 22 shows a chromatogram of mass scan (150-500 m/z) recorded in positive mode of ESI using Waters® MS3100 mass detector. The sample was obtained from microwave extraction at 130° C., 10 min. Retention times of CBD, CBDA, Δ⁹-THC, and THCA are shown. Single ion recording (SIR +ve) obtained for this sample shows the individual peaks representing CBD and Δ⁹-THC (FIG. 23) and CBDA and THCA (FIG. 24). If an analyte is absent or not detected in this mode, the expected retention time is shown.

FIG. 23 shows a Single Ion Recording (SIR +ve, m/z=315) to detect Cannabidiol (CBD) and Δ⁹-Tetrahydrocannabinol (Δ⁹-THC) in a sample obtained from microwave extraction at 130° C., 10 min (mass scan shown in FIG. 22).

FIG. 24 shows a single Ion Recording (SIR +ve, m/z=359) to detect Cannabidiolic acid (CBDA) and Tetrahydrocannabinolic acid (THCA) in a sample obtained from microwave extraction at 130° C., 10 min (mass scan shown in FIG. 22). Due to relatively high concentration of the analyzed sample the acidic forms of the CBD and THC are detected by the negative mode.

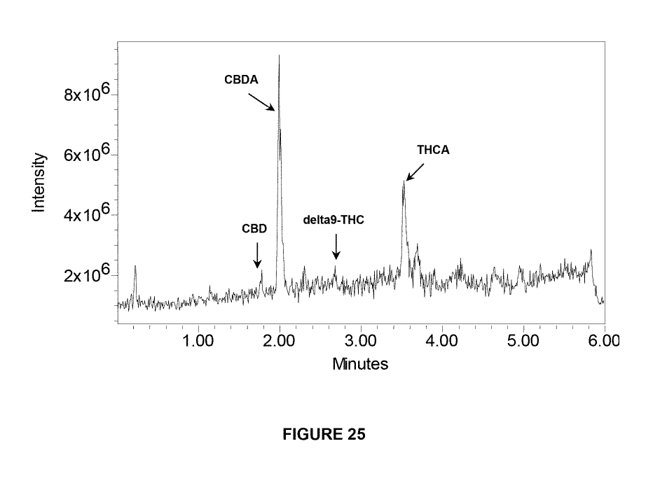

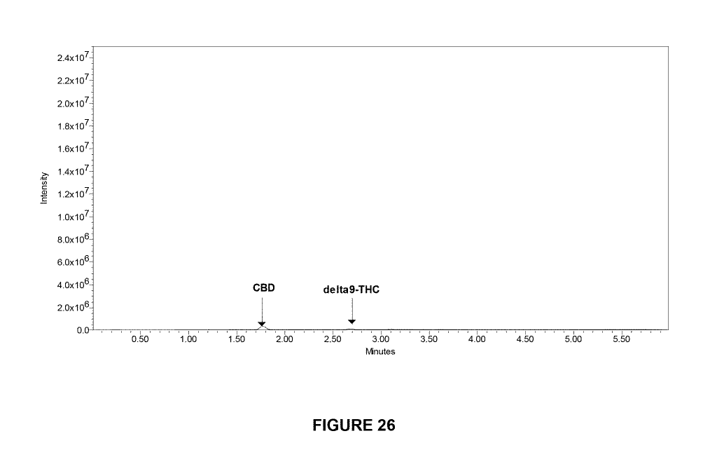

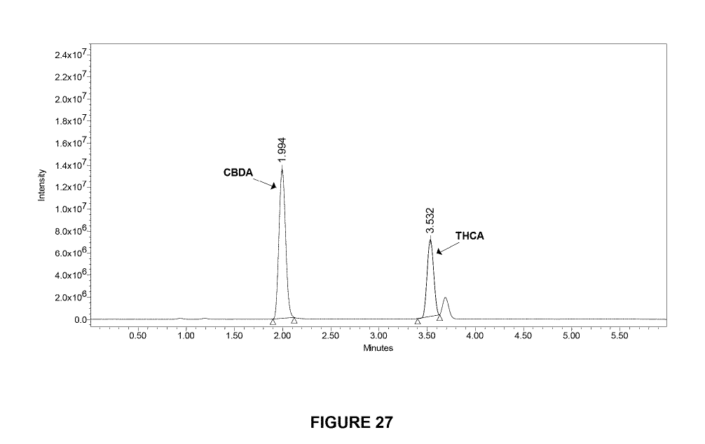

FIG. 25 shows a chromatogram of mass scan (150-500 m/z) recorded in negative mode of ESI using Waters® MS3100 mass detector. The sample was obtained from microwave extraction at 130° C., 10 min. Retention times of CBD, CBDA, Δ⁹-THC, and THCA are shown. Single ion recording (SIR −ve) obtained for this sample shows the individual peaks representing CBD and Δ⁹-THC (FIG. 26) and CBDA and THCA (FIG. 27). If an analyte is absent or not detected in this mode, the expected retention time is shown.

FIG. 26 shows a Single Ion Recording (SIR −ve, m/z=313) to detect Cannabidiol (CBD) and Δ⁹-Tetrahydrocannabinol (Δ⁹-THC) in a sample obtained from microwave extraction at 130° C., 10 min (mass scan shown in FIG. 25). Neutral cannabinoids (CBD and Δ⁹-THC are not visible in the negative mode detection as compared to the positive mode (FIG. 23).

FIG. 27 shows a Single Ion Recording (SIR −ve, m/z=357) to detect Cannabidiolic acid (CBDA) and Tetrahydrocannabinolic acid (THCA) in a sample obtained from microwave extraction at 130° C., 10 min (mass scan shown in FIG. 25). Some CBDA and THCA still present showing partial decarboxylation.

Summary of Results for Experiment 2

FIGS. 4-9 provide standard chromatography curves for Δ⁸-THC, Δ⁹-THC, THC-A, CBD, CBDA and CBN, respectively. These standard curves were generated for concentrations between 0 and 10 μg/mL and subsequently used for determining the concentration of each compound in various products formed using the embodiments described herein.

Table 5 shows the use of various solvents during ultrasonic extraction. There were also minimal amounts of cannabis plant material lost during the recovery filtration process (see Table 6). This also appears to be similar recovery across different extraction methods—solvent, Soxhlet, SFE extraction (See Tables 7 and 8).

When conducting cannabinoid extraction/decarboxylation in ethanol using a microwave, due to the boiling point of ethanol, it was shown that extraction/decarboxylation of cannabinoids using a microwave is best conducted at temperatures below 180° C., for example at 160° C.±10° C. (e.g. +/−the acceptable standard of error). It was also shown under the conditions used that conversion of THCA to THC (the desired decarboxylated product) was better at 130° C. than at 100° C., and that if conducted using microwave alone versus microwave and SFE, temperatures above 130° C., for example from 150° C. to 170° C. showed more efficient conversion. (See Tables 13 and 15 as well as FIG. 10). However, there appears to be significant loss of material if followed by SFE (see Tables 9 and 10, and 15). As such, the least number of steps and processes to obtain the desired results is the one-step extraction/decarboxylation method.

FIGS. 11 and 12 and Table 14 show that extraction resulted in minimal or no decarboxylated THC product, while the addition of the microwave step resulted in a significant increase in decarboxylated THC.

FIG. 13 shows concentrations of Δ⁹-THC, THC-A, CBD and CBN from cannabis after microwave radiation only at temperatures ranging between 100° C. and 170° C. FIG. 13 shows that microwave radiation at temperatures between 130° C. and 170° C. were optimal when ethanol was used as the solvent.

FIG. 14 shows concentrations of Δ⁹-THC, THC-A, CBD and CBN from the cannabis plant material after microwave radiation and subsequent SFE extraction at temperatures ranging between 100° C. and 170° C. Use of SFE extraction does not appear to significantly increase yield of decarboxylated cannabinoids.

FIG. 15 shows concentrations of total THC, CBD and CBN from the cannabis after microwave radiation and subsequent SFE extraction at a temperatures ranging between 100° C. and 170° C., where the acidic and neutral form of THC are added together to represent the total THC extracted by each method. Again, use of SFE extraction does not appear to significantly increase yield of decarboxylated cannabinoids.

From the above results, it appears that the significant decarboxylated product THC results from microwave extraction and that the addition of a second extraction step, such as SFE, does not appear to change the cannabinoid profile. It further shows that CBD as well as THC components are present in extract after microwave extraction alone or with microwave and SFE extractions combined.

In summary, the present disclosure shows that extraction and decarboxylation of cannabis plant material can be done concurrently using a microwave, set at a temperature below the boiling point of the extraction solvent, such as ethanol, without the need for a separate extraction step. This optimizes decarboxylated cannabinoid formation and recovery and can produce a more consistent and reproducible product with consistent and reproducible efficacy and therapeutic results.

An extraction step can also be included before use of a microwave. Although an extraction step after the microwave step is possible, it is not necessary.

### Experiment 3: Supercritical Fluid Extraction (SFE)

General Procedure:
1. Dried plant material was weighed and macerated using a mortar and pestle
2. Crushed plant material was transferred to a 10 mL extraction vessel and subjected to either of the following conditions below
3. Fractions from each run were collected every 5 mins and combined and concentrated to dryness (at 25° C.) then weighed (green resin)
4. Extraction was done three times with same plant fibre SFE conditions:

| Solvent A = CO₂ | Solvent B = ethanol | |
|---|---|---|
| Temperature = 25° C. | BPR = 12 MPa | PDA = 200-600 nm |
| | | (monitoring at 254 nm) |

Flow rate = 10 mL/min; make up pump = 1 mL/min

| | |
|---|---|
| Acquisition time = 30 mins | Method time = 30.2 mins |

i. 0.1 min-25 mins; gradient of 0-50% B in A
   26 mins; 100% A
   30 mins; 100% A

Table 16 below shows the corresponding results.

TABLE 16
Weights of extract after SFE.

| Strain | Run | Extract isolated (g) |
|---|---|---|
| Strain 1 = 1.0023 g | 1 | 0.2634 |
| (THC: 7.18/CBD: 8.6) | 2 | 0.0038 |
| | 3 | 0.010 |

### Experiment 4: Microwave-Assisted Decarboxylation of Extract with Ethanol (MAE)

General Procedure:
1. Extract isolated from the SFE method described above was dissolved in 5-10 mL ethanol and an appropriate volume transferred to 5 mL microwave vials
2. Added additional volume of ethanol and a stir bar to the 5 mL microwave vials
3. The vials were sealed and subjected to one of two microwave conditions below:
   (a) Temperature=150° C.; run time=10 mins; stir rate=600 rpm; absorption=Normal
   (b) Temperature=100° C.; run time=30 mins; stir rate=600 rpm; absorption=Normal
4. The solution was then concentrated at 35° C. after transferring to 20 mL vials

The results are shown in Table 17 below.

TABLE 17
Weights of the resins after subjecting to microwave heating.

| | | Amount of resin | Extract isolated |
|---|---|---|---|
| Strain | Method | used (g) | after microwave (g) |
| Strain 1 | (a) | 0.0832 | 0.0212 |
| (THC: 7.18/CBD: 8.6) | (b) | 0.0832 | 0.0612 |
| | (a) | 0.2095 | 0.1589 |
| Strain 2 | (a) | 0.1693 | 0.1114 |
| (THC: 0/CBD: 9) | | | |
| Strain 3 | (a) | 0.2722 | 0.1759 |
| (THC: 18.6/CBD: 0) | | | |

### Experiment 5: Additional Microwave-Assisted Extractions with Ethanol (MAE), Optimization of Conditions for Decarboxylation

General Procedure:
1. Dried plant material was weighed and macerated using a laboratory blender at 22,000 rpm for 60 secs
2. Crushed plant material was re-weighed and transferred to a 20 mL microwave vial along with a stir bar
3. Ethanol (10 mL) was added to the vial which was then sealed and subjected to the microwave conditions below:
   (a) Temperature=170° C.; run time=15 mins; pre-stirring=30 sec; stir rate=900 rpm; absorption=Normal
   (b) Temperature=150° C.; run time=20 mins; pre-stirring=30 sec; stir rate=900 rpm; absorption=Normal
4. The suspension was filtered and the filtrate and plant fibre collected separately
5. Filtrate was then concentrated at 35° C., then transferred to a 20 mL vial using ethanol and again concentrated at 35° C., then stored in the refrigerator

The results are shown in Table 18 below.

TABLE 18

| | | | | Extract |
|---|---|---|---|---|
| | | Amount of | Amount of | isolated |
| | | plant before | plant after | after |
| | | maceration | maceration | microwave |
| Strain | Method | (g) | (g) | (g) |
| Strain 1 | (a) | 1.0062 | 0.8256 | 0.2265 |
| (THC: 7.18/CBD: | | | | |
| 8.6) | | | | |
| Strain 2 | (a) | 1.0042 | 0.8382 | —^{a} |
| (THC: 0/CBD: 9) | (b) | 1.0050 | 0.8293 | 0.2155 |
| Strain 3 | (b) | 1.0063 | 0.7883 | 0.1166 |
| (THC: 18.6/CBD: 0) | | | | |

^{a}Desired temperature could not be achieved due to pressure build-up; cap of vial popped off causing solvent and plant fibre to escape from vial.

Since condition (b) in step 3 above proved successful at that scale, subsequent decarboxylations were performed using that microwave condition and were done in triplicate. However, the following modifications were made:
Step 1: Blend at 18,000 rpm for 4 secs
Step 4: Filtration done over celite/activated carbon (as previously described in an earlier report)
Step 5: Filtrate was then concentrated at 35° C., weighed, transferred to a 20 mL vial using ethanol, then stored in the refrigerator

The results are shown in Table 19 below.

TABLE 19

| | | | Amount of | | Average |
|---|---|---|---|---|---|
| | | Amount of | plant after | | Extract |
| | | plant before | maceration | Extract | isolated (g) ± |
| Strain | Runs | maceration (g) | (g) | isolated (g) | SD |
| Strain 1 | 1 | 1.0079 | 0.9260 | 0.2044 | 0.2105 ± 0.007 |
| (THC: | 2 | 1.0084 | 0.9594 | 0.2184 | |
| 7.18/CBD: | 3 | 1.0080 | 0.9254 | 0.2088 | |
| 8.6) | | | | | |
| Strain 2 | 1 | 1.0032 | 0.8287 | 0.1624 | 0.1654 ± 0.006 |
| (THC: | 2 | 1.0038 | 0.8467 | 0.1721 | |
| 0/CBD: 9) | 3 | 1.0040 | 0.8585 | 0.1617 | |
| Strain 3 | 1 | 1.0017 | 0.9169 | 0.2225 | 0.2212 ± 0.014 |
| (THC: | 2 | 1.0054 | 0.8952 | 0.2068 | |
| 18.6/CBD: 0) | 3 | 1.0033 | 0.9021 | 0.2343 | |

This experiments shows the consistency of the extraction method.

### Experiment 6: Cannabis Extractions (1 Gram Scale)

General Procedure (˜1.0 g Batch; Before Maceration):
1. Dried plant material was weighed and macerated using a laboratory blender at 18,000 rpm for 4 secs
2. Crushed plant material was re-weighed (˜0.875 g) and transferred to a 20 mL microwave vial along with a stir bar
3. Ethanol (10 mL) was added to the vial which was then sealed and subjected to the microwave conditions below:
   Temperature=150° C.; run time=20 mins; pre-stirring=30 sec; stir rate=900 rpm; absorption=Normal
4. The suspension was filtered over celite/activated carbon and the filtrate and plant fibre collected separately
5. Filtrate was then concentrated at 35° C., then transferred to a 20 mL vial using ethanol and again concentrated at 35° C., then stored in the refrigerator
6. Decarboxylations were performed in triplicate

Winterization Procedure:
1. Resin was dissolved in ethanol (10 mL/g) and heated at 40° C. for 5 mins in a water bath
2. Vial containing extract solution was cooled to −75° C. using a dry ice/acetone bath for 3-4 hrs
3. Solution was filtered with a pre-weighed syringe filter in 20 mL vials Filter specifications: Millex®-GV (sterile), Low Protein Binding Durapore® (PVDF) Membrane; 0.22 μm pore size; 33 mm diameter
4. Filter was washed with ethanol that had been cooled for 5 mins at −75° C. using a dry ice/acetone bath
5. Filtrate was concentrated at 35° C. and extract was weighed
6. Syringe filter was weighed after 2-3 days drying in the fumehood

TABLE 20
The quantities of cannabis plants used and the amounts of extract
obtained before and after winterization.

| | | | | Extract isolated | Extract |
|---|---|---|---|---|---|
| | | Plant before | Plant after | before | isolated after |
| | | maceration | maceration | winterization | winterization |
| Strain | Runs | (g) | (g) | (g) | (g) |
| Strain 1 | 1 | 1.008 ± 0.0003 | 0.9369 ± 0.019 | 0.2105 ± 0.007 | 0.1962 ± 0.007 |
| (THC: 7.18/CBD: | 2 | | | | |
| 8.6) | 3 | | | | |
| Strain 2 | 1 | 1.004 ± 0.0004 | 0.8446 ± 0.015 | 0.1654 ± 0.006 | 0.1541 ± 0.004 |
| (THC: 0/CBD: 9) | 2 | | | | |
| | 3 | | | | |
| Strain 3 | 1 | 1.003 ± 0.0019 | 0.9047 ± 0.011 | 0.2212 ± 0.014 | 0.2095 ± 0.009 |
| (THC: 18.6/CBD: | 2 | | | | |
| 0) | 3 | | | | |

TABLE 21
The quantities of cannabinoids (as % of resin and mg/g of plant) in
the extracts isolated, before and after winterization.

| | | Cannabinoid in resin before | | | | Cannabinoid in resin after | | | |
|---|---|---|---|---|---|---|---|---|---|
| | | winterization | | | | winterization | | | |
| | | | | Δ⁹- | Δ⁹- | | | Δ⁹- | Δ⁹- |
| | | CBD | CBD | THC | THC | CBD | CBD | THC | THC |
| Strains | Runs | (%) | (mg/g) | (%) | (mg/g) | (%) | (mg/g) | (%) | (mg/g) |
| Strain 1 | 1 | n.d. | n.d. | n.d. | n.d. | n.d. | n.d. | n.d. | n.d. |
| (THC: 7.18/ | 2 | | | | | | | | |
| CBD: 8.6) | 3 | | | | | | | | |
| Strain 2^{b} | 1 | n.d. | n.d. | | | 37.6 ± | 67.1 ± | 1.9 ± | 3.5 ± |
| (THC: 0/ | 2^{a} | | | | | 4.1 | 7.1 | 0.1 | 0.2 |
| CBD: 9) | 3^{a} | | | | | | | | |
| strain 3^{b} | 1 | | | n.d. | n.d. | | | 49.8 ± | 119.0 ± |
| (THC: 18.6/ | 2^{a} | | | | | | | 0.8 | 1.4 |
| CBD: 0) | 3^{a} | | | | | | | | |

n.d. = not determined, samples were used up before re-analysis with internal standard was performed
^{a}Average calculations based on runs 2 and 3 only.

Summary of Results:

Extracting and decarboxylating 1 gram scale batch of cannabis was successful.

### Experiment 7: Larger Scale MAE (˜3.75 g Batch; Before Maceration) Using the Modified Conditions

1. Dried plant material was weighed (˜3.75 g per batch) and macerated using a laboratory blender at 18,000 rpm for 4 secs.
2. Crushed plant material was re-weighed and transferred to 3×20 mL microwave vials along with stir bars (˜1.2 g of plant fibre per vial)
3. 95%-100% Ethanol (12 mL) was added to the vial which was then sealed and subjected to the microwave conditions below:
   Temperature=150° C.; run time=30 mins; pre-stirring=30 sec; stir rate=900 rpm; absorption=Normal
4. Note: Time for decarboxylation was increased to 30 mins since analysis revealed that decarboxylation was incomplete at that scale
5. The 3×20 mL vials were combined after decarboxylation and the suspension was filtered and the filtrate and plant fibre collected separately
6. Filtrate was then concentrated at 35° C., then transferred to a 20 mL vial using ethanol and again concentrated at 35° C., weighed and stored in the refrigerator
7. Decarboxylations were performed in duplicate

The results are shown in Table 22 below.

TABLE 22

| | | Amount of | | | |
|---|---|---|---|---|---|
| | | plant | Amount of | | |
| | | before | plant after | Extract | Average |
| | | maceration | maceration | isolated | Extract isolated |
| Strain | Runs | (g) | (g) | (g) | (g) ± SD |
| Strain 1 | 1 | 3.7922 | 3.6268 | 0.9967 | 0.9437 ± 0.075 |
| (THC: | 2 | 3.7228 | 3.5800 | 0.8906 | |
| 7.18/ | | | | | |
| CBD: | | | | | |
| 8.6) | | | | | |
| Strain 2 | 1 | —^{b} | 1.2427 | —^{c} | —^{c} |
| (THC: | | | | | |
| 0/ | | | | | |
| CBD: | | | | | |
| 9) | | | | | |
| Strain 3 | 1 | 3.7100 | 3.5978 | 1.0658 | 1.1572 ± 0.1293 |
| (THC: | 2 | 3.8012 | 3.6035 | 1.2486 | |
| 18.6/ | | | | | |
| CBD: | | | | | |
| 0) | | | | | |

^{b}Plant fibre is supplied as pulverized buds. Size was appropriate, therefore no further maceration was done.
^{c}Desired temperature of 150° C. could not be achieved due to pressure build-up.

Summary of Results:

From the results above, it can be concluded that larger scale microwave assisted extraction was successful, at 3-4 grams scale. This method can be scaled up into multi-gram and larger scales with appropriate adjustments to conditions.

### Experiment 8: Large Scale Microwave-Assisted Extractions with Ethanol (MAE)

(A) General Procedure (˜3.75 g Batch; Before Maceration):
1. Dried plant material was weighed and macerated using a laboratory blender at 18,000 rpm for 4 secs
2. Crushed plant material was re-weighed and transferred to 3×20 mL microwave vials along with stir bars (˜1.2 g of plant fibre per vial)
3. 95%-100% Ethanol (12 mL) was added to the vial which was then sealed and subjected to the microwave conditions below:
   Temperature=150° C.; run time=30 mins; pre-stirring=30 sec; stir rate=900 rpm; absorption=Normal
4. Note: Time for decarboxylation was increased to 30 mins since analysis revealed that decarboxylation was incomplete at that scale
5. All 20 mL vials were combined after decarboxylation and the suspension was filtered and the filtrate and plant fibre collected separately (first batch)
6. Filtrate was then concentrated at 35° C., then transferred to a 20 mL vial using ethanol and again concentrated at 35° C., weighed and stored in the refrigerator
7. Decarboxylations were performed in duplicate

Winterization Procedure:
1. Same as previously described

TABLE 23
The quantities of cannabis plants used and the amounts of extract
obtained before and after winterization.

| | | Plant | | | | Extract isolated | |
|---|---|---|---|---|---|---|---|
| | | before | Plant after | Extract isolated | | after | |
| | | maceration | maceration | before | | winterization | |
| Strain | Runs | (g) | (g) | winterization (g) | | (g) | |
| Strain 1 | 1^{a} | 3.7575 ± | 3.6034 ± | 0.9967 | 0.9437 ± | 0.7794 | |
| (THC: 7.18/ | 2 | 0.049 | 0.033 | 0.8906 | 0.075 | | |
| CBD: 8.6) | | | | | | | |
| Strain 2 | 1 | 1.2427 | b | n.d. | n.d. | n.d. | n.d. |
| (THC: 0/ | | | | | | | |
| CBD: 9) | | | | | | | |
| Strain 3 | 1 | 3.7556 ± | 3.6007 ± | 1.0618 | 1.1543 ± | 0.7984 | 0.8973 ± |
| (THC: 18.6/ | 2 | 0.064 | 0.004 | 1.2468 | 0.131 | 0.9961 | 0.140 |
| CBD: 0) | | | | | | | |

n.d. = not determined. Desired temperature of 150° C. could not be achieved due to pressure build-up; nothing further was done with buds.
^{a}No winterization was performed on run 1; winterization done on run 2 only.
b Plant fibre is supplied as pulverized buds. Size was appropriate, therefore no further maceration was done.

TABLE 24
The quantities of cannabinoids (as % of resin and mg/g of plant) in
the extracts isolated, before and after winterization.

| | | Cannabinoid in resin before | | | | Cannabinoid in resin after | | | |
|---|---|---|---|---|---|---|---|---|---|
| | | winterization | | | | winterization | | | |
| | | | | Δ⁹- | Δ⁹- | | | Δ⁹- | Δ⁹- |
| | | CBD | CBD | THC | THC | CBD | CBD | THC | THC |
| Strains | Runs | (%) | (mg/g) | (%) | (mg/g) | (%) | (mg/g) | (%) | (mg/g) |
| Strain 1 | 1 | n.d. | n.d. | n.d. | n.d. | 39.0 ± | 84.9 ± | 30.3 ± | 66.1 ± |
| (THC: 7.18/ | 2^{a} | | | | | 2.2 | 4.7 | 1.4 | 3.0 |
| CBD: 8.6) | | | | | | | | | |
| Strain 3 | 1 | | | 68.6 ± | 219.0 ± | | | 61.1 ± | 153.2 ± |
| (THC: 18.6/ | 2 | | | 8.4 | 2.7 | | | 4.6 | 35.0 |
| CBD: 0) | | | | | | | | | |

n.d. = not determined; samples were used before re-analysis with internal standard was performed.
^{a}Calculations based on run 2 only.

(B) General Procedure (˜7.5 g Batch; Before Maceration):
1. Dried plant material was weighed and macerated using a laboratory blender at 18,000 rpm for 10 secs
2. Crushed plant material was re-weighed and transferred to 6×20 mL microwave vials along with stir bars (˜1.2 g of plant fibre per vial)
3. See steps 3-6 in (A) above

Winterization Procedure:
1. Same as previously described

TABLE 25
The quantities of Variety 1 (THC: 7.18/CBD: 8.6) used and the
amounts of extract obtained before and after winterization.

| | | | Extract isolated | Extract |
|---|---|---|---|---|
| | Amount of | Amount of | before | isolated after |
| | plant before | plant after | winterization | winterization |
| Runs | maceration (g) | maceration (g) | (g) | (g) |
| 1 | 7.5361 | 3.6156 ± 0.002 | 1.0154 ± 0.064 | 0.8115 ± 0.030 |
| 2 | | | | |
| 3 | 7.5063 | 3.6640 ± 0.026 | 0.9327 ± 0.014 | 0.8247 ± 0.022 |
| 4 | | | | |

TABLE 26
The quantities of cannabinoids (as % of resin and mg/g of plant) in
the Variety 1 extract isolated, before and after winterization.

| | Cannabinoid in resin before | | | | Cannabinoid in resin after | | | |
|---|---|---|---|---|---|---|---|---|
| | winterization | | | | winterization | | | |
| | | | Δ⁹- | Δ⁹- | | | Δ⁹- | Δ⁹- |
| | CBD | CBD | THC | THC | CBD | CBD | THC | THC |
| Runs | (%) | (mg/g) | (%) | (mg/g) | (%) | (mg/g) | (%) | (mg/g) |
| 1 | 40.6 ± | 113.7 ± | 31.1 ± | 87.3 ± | 31.6 ± | 70.8 ± | 23.1 ± | 51.6 ± |
| 2 | 3.9 | 3.7 | 2.0 | 0.1 | 5.5 | 9.7 | 4.1 | 7.3 |
| 3 | 29.2 ± | 74.3 ± | 20.9 ± | 53.3 ± | 44.0 ± | 99.0 ± | 31.3 ± | 70.3 ± |
| 4 | 0.1 | 0.9 | 1.0 | 3.0 | 2.9 | 4.7 | 0.7 | 0.2 |

(A) General Procedure (˜4.0 and 7.0 g Batches; Before Maceration):
1. Dried plant material was weighed and macerated using a laboratory blender at 18,000 rpm for 10 secs
2. Crushed plant material was re-weighed and transferred to 20 mL microwave vials along with stir bars (˜1.2 g of plant fibre per vial)
3. For ˜4.0 g batch, see steps 3-5 in (A) above
4. For ˜7.0 g batch, see steps 3-5 in (A) above

Winterization Procedure:
1. Same as previously described

TABLE 27
The quantity of Variety 1 plant (THC: 7.18/CBD: 8.6) used and the
amount of extract obtained before and after winterization.

| | Amount | | Extract isolated | Extract isolated |
|---|---|---|---|---|
| | of plant | Amount of | before | after |
| | before | plant after | winterization | winterization |
| Runs | maceration (g) | maceration (g) | (g) | (g) |
| 1 | 4.0325 | 3.8199 | 0.8889 | 0.7358 |
| 2 | 7.1293 | 6.8717 | 1.5244 | 1.4101 |

TABLE 28
The quantities of cannabinoids (as % of resin and mg/g of plant) in
the Variety 1 extract isolated, before and after winterization.

| | Cannabinoid in resin before | | | | Cannabinoid in resin after | | | |
|---|---|---|---|---|---|---|---|---|
| | winterization | | | | winterization | | | |
| | | | Δ⁹- | Δ⁹- | | | Δ⁹- | Δ⁹- |
| | CBD | CBD | THC | THC | CBD | CBD | THC | THC |
| Runs | (%) | (mg/g) | (%) | (mg/g) | (%) | (mg/g) | (%) | (mg/g) |
| 1 | 30.2 ± | 70.3 ± | 27.1 ± | 63.0 ± | 38.4 ± | 73.9 ± | 32.1 ± | 61.8 ± |
| | 0.0 | 0.1 | 0.4 | 0.0 | 6.1 | 11.8 | 7.5 | 14.4 |
| 2 | 33.6 ± | 74.5 ± | 28.9 ± | 64.2 ± | 42.5 ± | 87.2 ± | 37.0 ± | 75.9 ± |
| | 2.9 | 6.5 | 3.2 | 7.1 | 0.5 | 1.1 | 2.9 | 5.9 |

Summary of Results:

Extracting and decarboxylating 3.75 gram scale batch of cannabis was successful.

### Experiment 9: Larger Scale Microwave-Assisted Extractions with Ethanol (MAE) and Winterization

1. Dried plant material was weighed (˜7.5 g per batch) and macerated using a laboratory blender at 18,000 rpm for 10 secs
2. Crushed plant material was re-weighed and transferred to 6×20 mL microwave vials along with stir bars (˜1.2 g of plant fibre per vial)
3. 95%-100% Ethanol (12 mL) was added to the vial which was then sealed and subjected to the microwave conditions below:
   Temperature=150° C.; run time=30 mins; pre-stirring=30 sec; stir rate=900 rpm; absorption=Normal
4. The 3×20 mL vials were combined after decarboxylation and the suspension was filtered and the filtrate and plant fibre collected separately
5. Filtrate was then concentrated at 35° C., then transferred to a 20 mL vial using ethanol and again concentrated at 35° C., weighed and stored in the refrigerator
6. Resin was dissolved in ethanol (10 mL/g)
7. Vials containing extract solution were cooled to −75° C. using a dry ice/acetone bath for 4 hrs
8. Solution was filtered with a pre-weighed syringe filter in 20 mL vials
9. Filter specifications: Millex®-GV (sterile), Low Protein Binding Durapore® (PVDF) Membrane, 0.22 μm pore size, 33 mm diameter
10. Filter was washed with ethanol that had been cooled for 5 mins at −75° C. using a dry ice/acetone bath
11. Filtrate was concentrated at 35° C. and vacuum dried for 2 days at 40° C. (via water bath), then extract was weighed
12. Syringe filter was weighed after 2-3 days drying in the fumehood

The results are shown in Table 29 below.

TABLE 29

| | | Amount of | | | | |
|---|---|---|---|---|---|---|
| | | plant | Amount of | | mg of | |
| | | before | plant after | extract | cannabinoid/g | |
| | | maceration | maceration | isolated | of plant (%) | |
| Strain | Runs | (g) | (g) | (g) | THC | CBD |
| Strain 1 | 1 | 7.5036 | 3.682 | 0.8403 | 7.0 | 10.7 |
| (THC: 7.18/ | 2 | | 3.6459 | 0.8091 | 7.7 | 12.2 |
| CBD: 8.6) | | | | | | |

Summary of Results:

7.5 g large scale batch of cannabis extraction and decarboxylation was successful. These experiments demonstrate that the methods of the disclosure consistently extract and decarboxylate cannabinoids and can be used on a commercial scale.

### Experiment 10: Winterization

Winterization is a procedure typically used to remove waxes and other partially soluble materials at 0±10° C. temperature range. This process may not be applicable, if there are no waxes present in the extract, or such hydrophobic molecules are broken down, and would be solidify at the ice-bath or below-zero temperatures.

### Experiment 10A

To remove waxes, the solutions of extract which had been stored in the refrigerator (−5° C.) for 1-3 weeks were manipulated as follows:
1. Solution was filtered using a syringe filter in 20 mL vials
   Filter specifications: Millex®-GV (sterile)
   Low Protein Binding Durapore® (PVDF) Membrane
   0.22 μm pore size
   13 mm diameter
2. Filtrate was concentrated at 35° C. and vacuum dried for 3 days at 40° C. (via water bath)
3. Extracts were then re-weighed

### Experiment 10B

1. Resin was dissolved in 95%-100% ethanol (10 mL/g) and heated at 40° C. for 5 mins in a water bath
2. Vial containing extract solution was cooled to −75° C. using a dry ice/acetone bath for 3-3.75 hrs
3. Solution was filtered with a pre-weighed syringe filter in 20 mL vials (see above for filter specifications); filter was washed with ethanol that had been cooled for 5 mins at −75° C. using a dry ice/acetone bath
4. Filtrate was concentrated at 35° C. and extract was weighed
5. Syringe filter was weighed after 2-3 days drying in the fumehood

The results are shown in Tables 30a and b below.

TABLE 30a
Amount of extract isolated before and after winterization methods

| | | Before 1^{st} | After 1^{st} | After 2^{nd} |
|---|---|---|---|---|
| | | winterization | winterization | winterization |
| Strain | Runs | (g) | and drying (g) | and drying (g) |
| Strain 1 | 1 | 0.2044 | 0.2111 | 0.2031 |
| (THC: 7.18/CBD: | 2 | 0.2184 | 0.2345 | 0.1964 |
| 8.6) | 3 | 0.2088 | 0.1981 | 0.1891 |
| Strain 2 | 1 | 0.1624 | 0.1677 | 0.1580 |
| (THC: 0/CBD: 9) | 2 | 0.1721 | 0.1622 | 0.1508 |
| | 3 | 0.1617 | 0.1677 | 0.1536 |
| Strain 3 | 1 | 0.2225 | 0.2088 | 0.1996 |
| (THC: 18.6/CBD: | 2 | 0.2068 | 0.2221 | 0.2145 |
| 0) | 3 | 0.2343 | 0.2243 | 0.2145 |

TABLE 30b
Average extract isolated before and after winterization methods

| | | Before 1st | After 1st | After 2nd |
|---|---|---|---|---|
| | | winterization | winterization | winterization |
| Strain | Runs | (g) | and drying (g) | and drying (g) |
| Strain 1 | 1 | 0.2105 ± 0.007 | 0.2146 ± 0.018 | 0.1962 ± 0.007 |
| (THC: | 2 | | | |
| 7.18/CBD: 8.6) | 3 | | | |
| Strain 2 | 1 | 0.1654 ± 0.006 | 0.1659 ± 0.003 | 0.1541 ± 0.004 |
| (THC: 0/CBD: | 2 | | | |
| 9) | 3 | | | |
| Strain 3 | 1 | 0.2212 ± 0.014 | 0.2184 ± 0.008 | 0.2095 ± 0.009 |
| (THC: | 2 | | | |
| 18.6/CBD: 0) | 3 | | | |

Tables 31a and b below show the amount of cannabinoid (in milligrams) in the extract per gram of plant material isolated before and after winterization methods

TABLE 31a
Amount of cannabinoid (in milligrams) in the
extract per gram pf plant material

| | | Before 1^{st} | | 1^{st} | | 2^{nd} | |
|---|---|---|---|---|---|---|---|
| | | winterization | | winterization | | winterization | |
| | | (mg/g) | | (mg/g) | | (mg/g) | |
| | | | Δ⁹- | | Δ⁹- | | Δ⁹- |
| Strains | Runs | CBD | THC | CBD | THC | CBD | THC |
| Strain 1 | 1 | 92.9 | 65.1 | 98.3 | 76.2 | 157.4 | 109.0 |
| (THC: | 2 | 114.9 | 82.3 | 114.3 | 91.5 | 124.7 | 93.7 |
| 7.18/ | 3 | 115.5 | 85.7 | 113.7 | 93.8 | 184.5 | 138.1 |
| CBD: 8.6) | | | | | | | |
| Strain 2 | 1 | 200.5 | | 113.7 | | 149.7 | |
| (THC: | 2 | 211.6 | | 109.5 | | 147.3 | |
| 0/CBD: 9) | 3 | 146.9 | | 131.2 | | 161.9 | |
| Strain 3 | 1 | | 406.1 | | 207.7 | | 235.4 |
| (THC: | 2 | | 185.6 | | 274.8 | | 236.2 |
| 18.6/ | 3 | 92.9 | 232.9 | 98.3 | 301.0 | 157.4 | 283.4 |
| CBD: 0) | | | | | | | |

TABLE 31b
Average cannabinoid (in milligrams) in the
extract per gram pf plant material

| | | Before 1^{st} | | 1^{st} | | 2^{nd} | |
|---|---|---|---|---|---|---|---|
| | | winterization | | winterization | | winterization | |
| | | (mg/g) | | (mg/g) | | (mg/g) | |
| Strains | Runs | CBD | Δ⁹-THC | CBD | Δ⁹-THC | CBD | Δ⁹-THC |
| Strain 1 | 1 | 107.8 ± | 77.7 ± | 108.8 ± | 87.2 ± | 155.5 ± | 113.6 ± |
| (THC: | 2 | 12.9 | 11.0 | 9.1 | 9.6 | 29.9 | 22.6 |
| 7.18/ | 3 | | | | | | |
| CBD: 8.6) | | | | | | | |
| Strain 2 | 1 | 186.3 ± | | 118.1 ± | | 153.0 ± | |
| (THC: 0/ | 2 | 34.6 | | 11.5 | | 7.8 | |
| CBD: 9) | 3 | | | | | | |
| Strain 3 | 1 | | 274.9 ± | | 261.2 ± | | 251.7 ± |
| (THC: | 2 | | 116.1 | | 48.1 | | 27.5 |
| 18.6/ | 3 | | | | | | |
| CBD: 0) | | | | | | | |

Summary of Results:

Winterization of extracts was successful in removing waxes from the extract.

### Experiment 11: Chemistry of Medicinal cannabis Before and after Decarboxylation

Methodology

Extraction:

Dried plant material (1 g) was weighed and transferred to a mortar and was macerated using a pestle. The crushed plant material was then transferred into a 10 mL vessel and was subjected to supercritical fluid extraction (SFE), with supercritical CO₂ as solvent A and ethanol as solvent B. The photodiode array detector was set to monitor wavelengths in the range of 200-600 nm and the back pressure regulator was set to 12 MPa. The SFE conditions used were: flow rate=10 mL/min (CO₂ and slave pumps) and 1 mL/min (make-up pump); temperature=25° C.; gradient: 100% A—50% A (0.1-25 mins), 100% B (25-26 mins) and 100% A (26-30 mins). Once the method was completed, all fractions were combined and concentrated to dryness under reduced pressure (at 25° C.) to afford 0.28 g of a green sticky resin. This was used for further work-up and analyses.

Activation:

Activation of phytocannabinoids was conducted by subjecting cannabis extract to heat using microwaves. A 5 mL-size microwave vial was charged with cannabis extract (27.72 mg) dissolved in ethanol (2 mL). The vial was sealed and was subjected to heat for 10 min at 150° C. in a pressure vessel to afford a green sticky extract. This was concentrated to dryness at 35° C. to obtain the activated cannabis extract as a resin (21.2 mg).

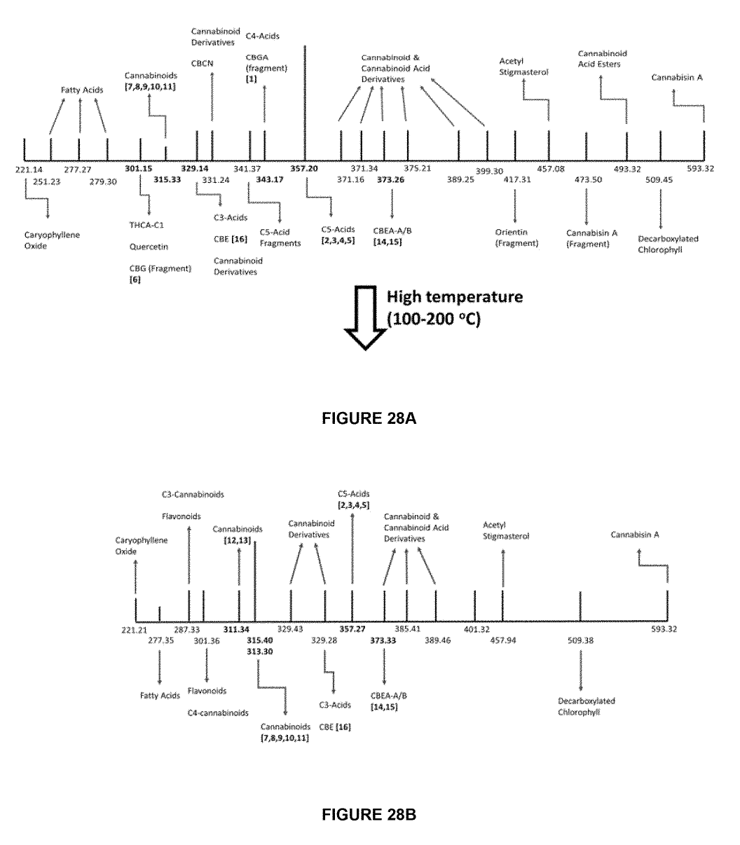

FIGS. 28A and 28B show the overview of select signals as seen in the mass spectra before (A) and after (B) decarboxylation of cannabis extract, as well as their corresponding compound classes. Bolded signals are compounds identified in the cannabinoid biosynthetic pathway.

Table 32.

Potential changes in chemical composition after decarboxylation of strain I cannabis extract. Left column indicates the potential compounds that were present in cannabis extract obtained through a supercritical fluid extraction (SFE), but not in the decarboxylated resin. This extract was then subjected to heating conditions using microwave technology, and the right column shows the new chemicals that were identified, which were not present in the cannabis extract prior to employing microwave technology described in this disclosure.

TABLE 32

| | New Compounds Found Upon |
|---|---|
| Compounds Lost Upon Activation | Activation |
| Roughanic acid (Fatty acid) | Kaempferol (Flavonol) |
| α-Linolenic acid (Fatty acid) | Luteolin (Flavonol) |
| Quercetin (Flavonoid) | 4,7-Dimethoxy-1,2,5-trihydroxyphenanthrene |
| | (Non-cannabinoid) |
| 4,5-Dihydroxy-2,3,6-trimethoxy-9,10- | 5-Methyl-4-pentyl-2,6,2-trihydroxybiphenyl |
| dihydrophenanthrene (Non-cannabinoid) | (Non-cannabinoid) |
| Δ⁹-Tetrahydrocannabiorcolic acid (C₁- | 5-Methyl-4-pentylbiphenyl-2,2,6-triol (Non- |
| Cannabinoid Acid) | cannabinoid) |
| Cannabigerol (C₅-Neutral Cannabinoid) | Cannabichromevarin (C₃-Neutral Cannabinoid) |
| Cannabichromanon (Neutral Cannabinoid) | Cannabicyclovarin (C₃-Neutral Cannabinoid) |
| Cannabigerovarinic acid (C₅-Cannabinoid Acid) | Cannabidivarin (C₃-Neutral Cannabinoid) |
| 6,7-cis/trans-Epoxycannabigerol (Cannabinoid | Δ⁷-cis-iso-Tetrahydrocannabivarin (C₃-Neutral |
| Derivative) | Cannabinoid) |
| 7-Hydroxycannabichromane (Cannabinoid | Δ⁹-tetrahydrocannabivarin (C₃-Neutral |
| Derivative) | Cannabinoid) |
| C₄-Tetrahydrocannabinolic acid (C₄-Cannabinoid | Chrysoeriol (Flavone) |
| Acid) | |
| Cannabitriol (Neutral Cannabinoid) | C₄-Cannabidiol (C₄-Neutral Cannabinoid) |
| Cannabiripsol (Neutral Cannabinoid) | C₄-Tetrahydrocannabinol (C₄-Neutral |
| | Cannabinoid) |
| Cannabigerolic Acid (C₅-Cannabinoid Acid) | Cannabifuran (Neutral Cannabinoid) |
| 7R-Cannabicoumaronic Acid (Cannabinoid Acid) | Cannabinol (C₅-Neutral Cannabinoid) |
| Tetrahydrocannabinolic acid-8-one (Cannabinoid | Cannabinodiol (C₅-Neutral Cannabinoid) |
| Acid Derivative) | |
| 5-Acetoxy-6-geranyl-3-n-pentyl-1,4- | 10-oxo- Δ^{6a}-tetrahydrocannabinol (Cannabinoid |
| benzoquinone (Non-cannabinoid) | Derivative) |
| 4-Acetoxycannabichromene (Cannabinoid | 7R-Cannabicourmarone (Neutral Cannabinoid) |
| Derivative) | |
| Cannabielsoic Acid-α/β (Cannabinoid Acid) | Cannabichromanone-D (Neutral Cannabinoid) |
| 6,7-trans/cis-Epoxycannabigerolic acid | Cannabidiol Monoethylether (Cannabinoid |
| (Cannabinoid Acid Derivative) | Derivative) |
| Δ⁹-Tetrahydrocannabinolic acid + C₂H₂O | Cannabielsoic Acid-A/B (Cannabinoid Acid) |
| Orientin (Flavone) | Sesquicannabigerol (Cannabinoid Derivative) |
| 4-Terpenyl-Δ⁹-Tetrahydrocannabinolate | |
| (Cannabinoid Acid Ester) | |
| α-Terpenyl-Δ⁹-Tetrahydrocannabinolate | |
| (Cannabinoid Acid Ester) | |
| Bornyl/epi-bornyl-Δ⁹-Tetrahydrocannabinolate | |
| (Cannabinoid Acid Ester) | |
| α/β-Fenchyl Δ⁹-Tetrahydrocannabinolate | |
| (Cannabinoid Acid Ester) | |

Closed system, microwave extraction provided the simultaneous extraction and decarboxylation of the cannabinoids. In the native cannabis extract, 63 compounds could be observed (FIG. 28A), and in the decarboxylated cannabis extract, there could be up to 22 new compounds (Table 32). Up to 26 compounds from the resin were not present in the decarboxylated cannabis resin, but were present in the cannabis resin prior to decarboxylation step.

### Experiment 12: Solvent-Free Decarboxylated cannabis Resin

General Procedure

To remove the solvent from the decarboxylated resin, the following procedures are used: distillers or rotary evaporators are used to evaporate solvents employed in extraction and decarboxylation process, concentrate and obtain solvent-free decarboxylated resin. During the evaporation of solvent, a higher temperature than ambient temperature is used to facilitate faster evaporation of the solvent. In addition, a vacuum may be used to facilitate removal of solvent at lower pressure than the atmospheric pressure. In general, such processes are well established and known to those skilled in the art.

The resulting decarboxylated cannabis resin may comprise less than 5% solvent, or the resin can be solvent-free.

### II. Sublingual Rapidly Disintegrating Tablet Formulations Comprising Decarboxylated Cannabis Resin.

The present disclosure provides decarboxylated cannabis resin in a solid formulation and methods of making and using same. In an embodiment, the solid formulation is a rapidly disintegrating sublingual tablet formulation.

Sublingual formulations comprising the decarboxylated cannabis resin of the present invention may be beneficial in that they may (i) provide a faster route of absorption of the cannabinoids and other medicinal ingredients from the decarboxylated cannabis resin to the subject compared with absorption through the oral route via gastrointestinal tract; (ii) avoid degradation in the hostile environment of the gastrointestinal tract; (iii) be used at lower doses because they are not metabolized in the liver; and/or (iv) provide an alternative dosage form for patients who do not wish to, or unable to, smoke or vape.

Placing the sublingual formulation under the tongue of a subject may result in the absorption of the ingredients of the cannabis resin by the oral mucosal lining which comprises capillaries located in connective tissue beneath the epithelium. Once the ingredients are absorbed by the capillaries, they enter venous circulation.

Sublingual formulations avoid passage via the stomach, intestines and liver (first pass metabolism) before entering general circulation. Non-sublingual oral drugs are subjected to hostile environments of the gastrointestinal tract and liver. Sublingual formulations are generally only subject to enzymes found in saliva before absorption.

Development of rapidly disintegrating tablets from cannabis resin is challenging due to the physical properties of cannabis resin, which is a hydrophobic, viscous, and glue-like substance. A rapidly disintegrating tablet requires the tablet to be stable in its physical form until the tablet is consumed, and then disintegrate rapidly when placed under the tongue.

In an embodiment, sublingual formulations of decarboxylated cannabis resin can be formulated to disintegrate rapidly when in contact with a suitable fluid, such as phosphate buffered solution (PBS), saliva, or other similar natural or artificial fluids. In an embodiment, sublingual formulations of cannabis resin disintegrate within 60 seconds when in contact with a phosphate buffered saline (PBS). In a further embodiment, sublingual formulations of cannabis resin disintegrate in less than 60 seconds, less than 50 seconds, less than 40 seconds, less than 30 seconds, less than 25 seconds, less than 20 seconds, less than 15 seconds, less than 10 seconds, less than 5 seconds, or any integer or fraction in these ranges (for example 7.84 seconds) when in contact with PB S.

In one embodiment, a method is provided for producing a rapidly disintegrating tablet comprising decarboxylated cannabis resin which disintegrates within 60 seconds in PBS and has desirable levels of friability and hardness. In certain embodiments, the decarboxylated cannabis resin is dissolved in a pharmaceutically acceptable organic solvent such as ethanol. A sugar alcohol, such as mannitol, is dissolved in a pharmaceutically acceptable polar solvent such as water, and the solutions are mixed together. Optionally, sonication may be used. In an embodiment, the ratio of cannabis resin to mannitol in the rapidly disintegrating sublingual tablet formulations is between 1:3 and 1:8. In certain embodiments, the ratio of cannabis resin to mannitol in the formulation is 1:3, 1:4, 1:5, 1:6, 1:7, or 1:8, or any fraction thereof, for example, 1:6.33. In one embodiment, the ratio is 1:6.

In certain embodiments, the solvents are substantially or completely removed using lyophilization, spray drying, fluid bed drying, high vacuum, or other suitable method, in order to produce a powder. Following powderization, one or more pharmaceutically acceptable excipients may be added, including a suitable disintegrant such as cross-linked polyvinylpyrrolidone (e.g. Kollidon CL™) or croscarmellose sodium. Other excipients may optionally be added such as diluents, fillers, binding agents, releasing agents, or lubricants. In certain embodiments, the excipients are magnesium stearate and optionally microcrystalline cellulose. In certain embodiments, the rapidly disintegrating tablet may further comprise flavoring agents, taste-masking agents, colorants, or other excipients to enhance the taste, texture and flavor of such tablets. In certain embodiments, the tablet comprises about 2-18 wt % of decarboxylated cannabis resin, about 40-80 wt % of mannitol, about 10-50 wt % of disintegrant, and optionally about 0-25 wt % of other excipients. In further embodiments, the tablet comprises 7-11 wt % of decarboxylated cannabis resin; about 50-63 wt % of mannitol; about 11-18 wt % of disintegrant, wherein the disintegrant is crosslinked polyvinylpyrrolidone or croscarmellose sodium; and the other excipients are magnesium stearate at an amount of about 0.6-1.1 wt % of the tablet, and microcrystalline cellulose at an amount of about 0-20 wt % of the tablet.

The components may be triturated or mixed to produce a homogenous mixture, homogeneous solid solution or homogeneous solid suspension. In certain embodiments, the homogenous mixture, homogeneous solid solution or homogeneous solid suspension is compressed into a tablet at a pressure of up to about 1 ton. In other embodiments, the homogenous mixture, homogeneous solid solution or homogeneous solid suspension is compressed into a tablet at a pressure of about 0.05 to 0.6 ton. In still further embodiments, the homogenous mixture, homogeneous solid solution or homogeneous solid suspension is compressed into a tablet at a pressure of about 0.1 to 0.4 ton.

In certain embodiments, there is provided a rapidly disintegrating tablet comprising decarboxylated cannabis resin, comprising cannabinoids which are at least 50%, 60%, 70%, 80%, 90%, 95%, or 100% (or any integer or fraction in these ranges, for example 96.33%) decarboxylated. In certain embodiments, the tablet disintegrates rapidly when placed in contact with PBS, saliva, or other similar natural or artificial fluid; the tablet additionally having suitable levels of friability and hardness.

Friability describes the tendency of a solid substance to break into smaller pieces with handling or contact. Friability testing is a laboratory technique to test the durability of tablets during transit or handling. Friability is calculated as the percentage of weight lost by tablets due to mechanical action during a friability test. In the examples that follow, tablets were tested for friability on a Sotax® Friabilitor model F1 using the standard protocol, as described by the manufacturer. Similar protocols are also described in various text books such as in Remington: The Science and Practice of Pharmacy (22nd edition), ISBN: 0857110624.

In an embodiment, the sublingual formulations of decarboxylated cannabis resin have a friability of 5% or less. In certain embodiments, the friability is less than 4%, less than 3%, less than 2%, less than 1%, or 0%, or any fraction thereof, for example 0.87%.

Tablet hardness testing is a laboratory technique to test the breaking point and structural integrity of a tablet under conditions of storage, transportation and handling before usage. In the examples that follow, tablet hardness was tested using an Engineering Systems® C50 Hardness Tester following the standard protocol, as described by the manufacturer. Similar protocols are also described in various text books such as in Remington: The Science and Practice of Pharmacy (22nd edition), ISBN: 0857110624.

In certain embodiments, the tablet comprises mannitol and a disintegrant such as cross-linked polyvinylpyrrolidone (e.g. Kollidon CL™) or croscarmellose sodium. In an embodiment, the ratio of cannabis resin to mannitol is between 1:3 and 1:8. In certain embodiments, the ratio of cannabis resin to mannitol in the formulation is 1:3, 1:4, 1:5, 1:6, 1:7, or 1:8, or any fraction thereof, for example, 1:6.33. In one embodiment, the ratio is 1:6.

In certain embodiments, the tablet comprises one or more additional pharmaceutically acceptable excipients, such as diluents, fillers, binding agents, releasing agents, or lubricants. In an embodiment, the excipients are magnesium stearate and optionally microcrystalline cellulose. In certain embodiments, the rapidly disintegrating tablet may further comprise flavoring agents, taste-masking agents, colorants, or other excipients to enhance the taste, texture and flavor of such tablets. In certain embodiments, the tablet comprises about 2-18 wt % of decarboxylated cannabis resin, about 40-80 wt % of mannitol, about 10-50 wt % of disintegrant, and optionally about 0-25 wt % of other excipients. In further embodiments, the tablet comprises 7-11 wt % of decarboxylated cannabis resin; about 50-63 wt % of mannitol; about 11-18 wt % of disintegrant, wherein the disintegrant is crosslinked polyvinylpyrrolidone or croscarmellose sodium; and the other excipients are magnesium stearate at an amount of about 0.6-1.1 wt % of the tablet, and microcrystalline cellulose at an amount of about 0-20 wt % of the tablet.

In certain embodiments, the disclosure provides the use of a decarboxylated cannabis resin in the manufacture of a rapidly disintegrating tablet. In certain embodiments, the resin is at least 50%, 60%, 70%, 80%, 90%, or 100% (or any integer or fraction in these ranges, for example 96.33%) decarboxylated, or any integer or fraction thereof or between.

The tablets of the present disclosure may be used in the treatment, prevention, or amelioration of symptoms, ailments, or diseases, for which Cannabis is used.

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein. The term “resin” is used as a short form for “decarboxylated cannabis resin” in the Examples and Tables that follow.

### Experiment 13: Prototype Solid Formulations

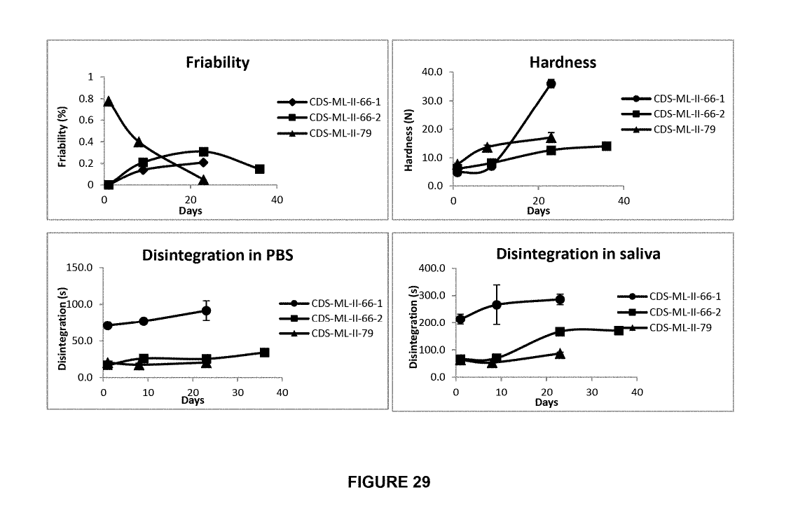

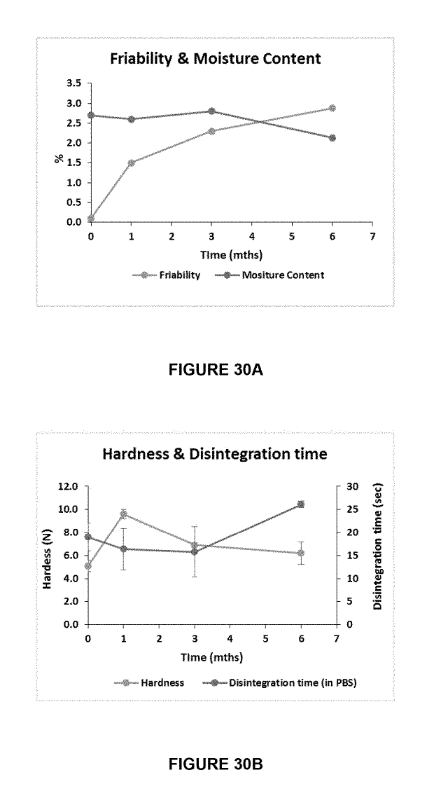

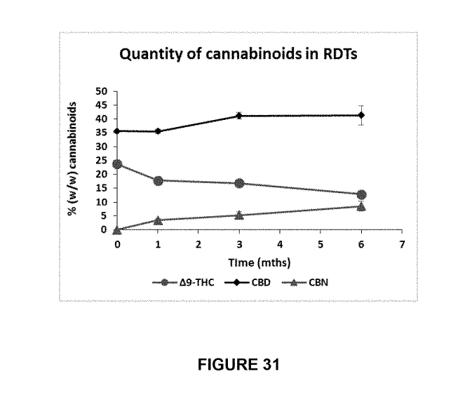

Different series of sublingual tablets formulations were prepared according to the ratios set out in Table 33, using different disintegrants. The standard operating procedures used were as follows:
1. In a porcelain mortar, dry powders of each formulation ingredients were well premixed using spatula/scoopula
2. The mixture powder was triturated to give a homogenous mixture
3. A reasonable amount of the mixture was loaded to the sample holder part of the die set (Special 3 mm tablet die from Specac®)
4. The remaining parts of the die were assembled in place and punched under 0.2 ton pressure using Sirius 0.5 ton Manually Operated Hydraulic Press, to afford a final sublingual tablet of an average of 35-36.5 mg
5. Approximately, 24 tablets of each formulation were obtained and stored in a 4 mL glass vial at room temperature (FIGS. 29 and 30) for further investigations

Different series of sublingual tablet formulations were prepared according to the ratios set out in Table 33, using different disintegrants. The procedures used were as follows:

TABLE 33
Components of sublingual tablet Formulations 1-15

| | | Microcrystalline | Magnesium | Sodium starch | Croscarmellose | Polyplasdone ™ |
|---|---|---|---|---|---|---|
| Formulations | Mannitol | cellulose* | stearate | glycolate | Sodium | crospovidone |
| 1 | 700 mg | 210 mg | 10.5 mg | — | — | — |
| 2 | 700 mg | 210 mg | 10.5 mg | 63 mg | — | — |
| 3 | 700 mg | 210 mg | 10.5 mg | — | 63 mg | — |
| 4 | 700 mg | 210 mg | 10.5 mg | 35 mg | — | — |
| 5 | 700 mg | 210 mg | 10.5 mg | 70 mg | — | — |
| 6 | 700 mg | 210 mg | 10.5 mg | 105 mg | — | — |
| 7 | 700 mg | 210 mg | 10.5 mg | 140 mg | — | — |
| 8 | 700 mg | 210 mg | 10.5 mg | — | 35 mg | — |
| 9 | 700 mg | 210 mg | 10.5 mg | — | 70 mg | — |
| 10 | 700 mg | 210 mg | 10.5 mg | — | 105 mg | — |
| 11 | 700 mg | 210 mg | 10.5 mg | — | 140 mg | — |
| 12 | 700 mg | 210 mg | 10.5 mg | — | — | 35 mg |
| 13 | 700 mg | 210 mg | 10.5 mg | — | — | 70 mg |
| 14 | 700 mg | 210 mg | 10.5 mg | — | — | 105 mg |
| 15 | 700 mg | 210 mg | 10.5 mg | — | — | 140 mg |

*Partially depolymerized from Spectrum Chemicals Company, of density and molecular weight equivalent to the brand Avicel ® PH 102

### Experiment 14: Disintegration Studies Using Prototype Solid Formulations

The procedure used was as follows:
1. In a 24 multi-well plate, 1 mL of Phosphate Buffer Saline (PBS) or distilled water (DW) was placed in 12 wells each
2. One tablet from each of the Formulations 24-35 was placed in a well of PBS and DW
3. A six minutes video was recorded for the entire experiment comparing the disintegration time and behavior of the 12 formulations in real time basis (head to head)

The results are shown below in Tables 34A and 34B. As shown below, croscarmellose sodium and Kollidon CL™ had faster disintegration times than sodium starch glycolate in PBS and in deionized water, suggesting that starch-based substances may increase disintegration time.

TABLE 34A
Disintegration times and conditions in PBS for each tablet from
Formulations 4-15

| | | Time on the video track | | |
|---|---|---|---|---|
| | Variable | Dropped-in at | Disintegrated | Disintegration |
| Formulations | Component | (mm:ss) | at (mm:ss) | time (sec) |
| 4 | 5% Na. starch | 00:28 | 01:02 | 34 |
| | glycolate | | | |
| 5 | 10% Na. starch | 00:36 | 01:10 | 34 |
| | glycolate | | | |
| 6 | 15% Na. starch | 00:43 | 01:18 | 35 |
| | glycolate | | | |
| 7 | 20% Na. starch | 00:51 | 01:45 | 54 |
| | glycolate | | | |
| 8 | 5% Croscarmellose | 00:57 | 01:10 | 13 |
| | sodium | | | |
| 9 | 10% Croscarmellose | 01:03 | 01:19 | 16 |
| | sodium | | | |
| 10 | 15% Croscarmellose | 01:09 | 01:27 | 18 |
| | sodium | | | |
| 11 | 20% Croscarmellose | 01:15 | 01:36 | 21 |
| | sodium | | | |
| 12 | 5% Kollidon CL | 01:22 | — | Incomplete |
| | | | | disintegration |
| 13 | 10% Kollidon CL | 01:30 | 01:41 | 11 |
| 14 | 15% Kollidon CL | 01:35 | 01:45 | 10 |
| 15 | 20% Kollidon CL | 01:41 | 01:54 | 13 |

TABLE 34B
Disintegration times and conditions in DW for each tablet from
Formulations 4-15

| | | Time on the video track | | |
|---|---|---|---|---|
| | | Dropped-in | Disintegrated | Disintegration |
| Formulations | Variable Component | at (mm:ss) | at (mm:ss) | time (sec) |
| 4 | 5% Na. starch glycolate | 01:49 | 02:16 | 27 |
| 5 | 10% Na. starch | 01:53 | 02:33 | 40 |
| | glycolate | | | |
| 6 | 15% Na. starch | 01:58 | 02:44 | 46 |
| | glycolate | | | |
| 7 | 20% Na. starch | 02:03 | 02:55 | 52 |
| | glycolate | | | |
| 8 | 5% Croscarmellose | 02:08 | 02:23 | 15 |
| | sodium | | | |
| 9 | 10% Croscarmellose | 02:13 | 02:31 | 18 |
| | sodium | | | |
| 10 | 15% Croscarmellose | 02:18 | 02:31 | 13 |
| | sodium | | | |
| 11 | 20% Croscarmellose | 02:23 | 02:42 | 19 |
| | sodium | | | |
| 12 | 5% Kollidon CL | 02:27 | — | Incomplete |
| | | | | disintegration |
| 13 | 10% Kollidon CL | 02:32 | 02:52 | 20 |
| 14 | 15% Kollidon CL | 02:38 | 02:52 | 14 |
| 15 | 20% Kollidon CL | 02:44 | 02:57 | 13 |

### Experiment 15: Sublingual Formulations Comprising Decarboxylated Cannabis Resin

Decarboxylated cannabis resin was prepared from cannabis plants of Strain 1 by microwave-assisted extractions with ethanol (MAE) and winterization, as described in Experiment 9.

In the following experiments (Formulations 16-18), various permutations of mixing the excipients and lyophilization were investigated to prepare the powder form for punching the tablets.

TABLE 35
Components of sublingual tablet Formulations 16-19

| | | | Mg | Kollidon | | No. of tablets |
|---|---|---|---|---|---|---|
| Formulations | Mannitol | MCC* | stearate | CL ® | Resin | formulated |
| 16 | 600 mg | 180 mg | 10 mg | 150 mg | 200.5 mg | 9 |
| 17 | 305.4 mg | 91.6 mg | 4.6 mg | 75.5 mg | 101.8 mg | 5 |
| 18 | 589.2 mg | 176.8 mg | 8.8 mg | 142.1 | 196.4 mg | 8 |
| 19 | 567.3 mg | 170.2 mg | 8.5 mg | 140.3 mg | 189.1 mg | 9 |

*Microcrystalline cellulose, partially depolymerized from Spectrum Chemicals Company, of density and molecular weight equivalent to the brand Avicel ® PH 102

The procedures used for making the solid Formulations 16-19 were as follows:

Formulation 16: (contains THC=2.19 mg/tab, and CBD=3.14 mg/tab)
1. The resin was dissolved in 1 mL ethanol followed by addition of mannitol dissolved in 1 mL water.
2. Mixture was concentrated then lyophilized overnight.
3. Add other excipients to the lyophilized mixture
4. Triturate in a porcelain mortar with a pestle until a homogeneous solid solution is obtained
5. Weigh 115 mg of the mixture for each tablet
6. Load the 115 mg powder to a 10 mm die
7. Punch under 0.4 ton pressure, using Specac® manual hydraulic press
8. Collect tablets in a 20 mL vial

Formulation 17: (contains THC=1.24 mg/tab, and CBD=1.57 mg/tab)
1. The resin was dissolved in 1 mL ethanol and a suspension of microcrystalline cellulose and mannitol in 4 mL water was added followed by 1 min sonication
2. Add 3 more mL of water
3. Lyophilize for 41 hrs
4. Add other excipients to the lyophilized mixture
5. Triturate in a porcelain mortar with a pestle until having a homogeneous solid solution
6. Weigh 96.6 mg of the mixture for each tablet
7. Load the 96.6 mg powder to a 10 mm die
8. Punch under 0.4 ton pressure, using Specac® manual hydraulic press
9. Collect tablets in a 20 mL vial

Formulation 18: (contains THC=2.04 mg/tab, and CBD=2.73 mg/tab)
1. The resin was dissolved in 1 mL ethanol and a suspension of microcrystalline cellulose and mannitol in 4 mL water was added followed by 1 min sonication
2. Add 3 more mL of water
3. Lyophilize for 41 hrs
4. Add other excipients to the lyophilized mixture
5. Triturate in a porcelain mortar with a pestle until having a homogeneous solid solution
6. Weigh 123.7 mg of the mixture for each tablet
7. Load the 123.7 mg powder to a 10 mm die
8. Punch under 0.4 ton pressure, using Specac® manual hydraulic press
9. Collect tablets in a 20 mL vial

Formulation 19: (contains THC=2.61 mg/tab, and CBD=3.50 mg/tab)
1. The resin was dissolved in 1 mL ethanol and a suspension of all ingredients in 4 mL water was added followed by 1 min sonication
2. Add 3 more mL of water
3. Lyophilize for 41 hrs
4. Triturate in a porcelain mortar with a pestle until having a homogeneous solid solution
5. Weigh 107.6 mg of the mixture for each tablet
6. Load the 107.6 mg powder to a 10 mm die
7. Punch under 0.4 ton pressure, using Specac® manual hydraulic press
8. Collect tablets in a 20 mL vial

TABLE 36
Properties of sublingual tablet Formulations 16-19

| | | | Disintegration time (s) in |
|---|---|---|---|
| Formulations | Friability (%) | Hardness (N) | 1 mL PBS |
| 16 | 0.17 | 2.79 | >6 min |
| 17 | 0.069 | 5.49 | 37 |
| 18 | 0.054 | 19.12 | 410 |
| 19 | 0.22 | 14.80 | 214 |

From Table 36, Formulation 17 and its preparation method are suitable for making sublingual RDT formulations.

### Experiment 16: Potential Excipients for Sublingual Formulations Comprising Decarboxylated Cannabis Resin

Due to the relatively homogeneous appearance of Formulation 17, the tablet excipient was substituted while keeping the methodology similar to that of Formulation 17 (except with 5 sec sonication). Excipients tested here such as CMC, HPC (both cellulose derivatives), chitosan are to evaluate their role in generating the powderized mixture of cannabis resin and mannitol, by enhancing solubility of cannabis resin prior to lyophilization.

TABLE 37
Components of sublingual tablet Formulations 20-22

| | | | | | Mg | Kollidon | | No. of tablets |
|---|---|---|---|---|---|---|---|---|
| Formulations | Mannitol | CMC | Chitosan | HPC | stearate | CL ® | Resin | formulated |
| 20 | 305.4 mg | 91.6 mg | — | — | 4.6 mg | 75.5 mg | 101.8 mg | 5 |
| 21 | 305.4 mg | — | 91.6 mg | — | 4.6 mg | 75.5 mg | 101.8 mg | 5 |
| 22 | 305.4 mg | — | — | 91.6 mg | 4.6 mg | 75.5 mg | 101.8 mg | 5 |

The procedure used for making Formulations 20-22 were as follows:
1. To an ethanolic resin solution (1.02 mL; 100 mg/mL), a suspension of CMC, Chitosan, or HPC, and mannitol in 4 mL water was added, followed by 5 sec sonication
2. Steps 2-9 were the same as for Formulation 17.

TABLE 38
Properties of sublingual tablet Formulations 20-22

| | | | Disintegration time |
|---|---|---|---|
| Formulations | Friability (%) | Hardness (N) | in 1 mL PBS |
| 20 | 0.00 | 9.87 | >6 min |
| 21 | 0.139 | 6.51 | >6 min |
| 22 | 0.172 | 20.3 | >6 min |

Excipients tested above increased the hardness of the tablets and disintegration time was not acceptable.

Using a slightly modified methodology from that used for Formulations 20-22, Formulation 23 was done with the list of ingredients shown in Table 39.

TABLE 39
Components of sublingual Formulation 23

| | | | | | | No. of |
|---|---|---|---|---|---|---|
| Formula- | | | Mg | Kollidon | | tablets |
| tion | Mannitol | MCC* | Stearate | CL ® | Resin | formulated |
| 23 | 305.4 mg | 91.6 | 4.6 mg | 75.5 mg | 101.8 | 5 |
| | | mg | | | mg | |

*Microcrystalline cellulose, partially depolymerized from Spectrum Chemicals Company, of density and molecular weight equivalent to the brand Avicel ® PH 102

Procedure for making Formulation 23:
1. Ethanolic resin solution (1.02 mL; 100 mg/mL) was diluted to half its concentration and a suspension of microcrystalline cellulose (MCC) and mannitol in 8 mL water was added, followed by 5 sec sonication
2. Steps 2-9 were the same as for Formulation 38

TABLE 40
Properties of sublingual tablet Formulation 23

| | | | Disintegration time in |
|---|---|---|---|
| Formulation | Friability (%) | Hardness (N) | 1 mL PBS |
| 23 | 0.00 | 9.87 | >6 min |

This experiment demonstrated that inclusion of MCC prior to the drying (lyophilization) step could increase the disintegration time.

### Experiment 17: Comparison of Sugar Alcohols for Sublingual Formulations Comprising cannabis Resin

TABLE 41
Formulations 24-26 comprising different sugar alcohols

| | | | | | Mg | Kollidon | | No. of tablets |
|---|---|---|---|---|---|---|---|---|
| Formulation | Mannitol | Sorbitol | MCC* | HPC | Stearate | CL ® | Resin | formulated |
| 24 | 305.4 mg | — | 46 mg | 46 mg | 4.6 mg | 75.5 mg | 101.8 mg | 5 |
| 25 | — | 305.4 mg | 46 mg | 46 mg | 4.6 mg | 75.5 mg | 101.8 mg | —^{a} |
| 26 | — | 305.4 mg | 92 mg | — | 4.6 mg | 75.5 mg | 101.8 mg | 5 |

*Microcrystalline cellulose, partially depolymerized from Spectrum Chemicals Company, of density and molecular weight equivalent to the brand Avicel ® PH 102
^{a}Tablets could not be made because formulation was a sticky, flake-like residue

Procedure for making Formulations 24-26:
1. To an ethanolic resin solution (1.02 mL; 100 mg/mL), the appropriate suspension of mannitol, sorbitol, HPC or MCC in 4 mL water was added
2. See Formulation 38 for steps 2-7
3. Punch under 0.3 ton pressure, using Specac® manual hydraulic press
4. Collect tablets in a 20 mL vial

TABLE 42
Properties of sublingual tablet Formulations 24-26

| | Friability | | |
|---|---|---|---|
| Formulations | (%) | Hardness (N) | Disintegration time in 1 mL PBS |
| 24 | 0.31 | 18.0 | >6 min |
| 25 | —^{a} | —^{a} | —^{a} |
| 26 | 0 | 9.5 | >6 min |

^{a}Tablets could not be made because formulation was a sticky, flake-like residue

These experiments indicate that inclusion of excipients such as sorbitol, HPC or MCC, or other sugar alcohols (other than mannitol) or other cellulose derivatives, prior to the drying (lyophilization) step may increase disintegration times and create sticky powders that are not suitable for RDT formulations.

Once the solvents are removed, the resulting mannitol and cannabis resin powder could be mixed or triturated with additional excipients such as the cellulose derivatives to increase lubrication and optimize other properties en route to the punching of tablets.

### Experiment 18: Evaluation of Excipient Concentrations and Sequence of Addition

Procedure for making Formulation 27-29:
1. Removed 0.51 mL of 200 mg/mL resin solution, then placed in vial and diluted to 1 mL with ethanol (102 mg of resin)
2. A solution of mannitol in 4 mL water was added
3. Added 3 more mL of water
4. Lyophilize for 41 hrs
5. Add other excipients to the lyophilized mixture
6. Triturate in a porcelain mortar with a pestle until having a homogeneous solid solution
7. Weigh ˜96.5 mg of the mixture for each tablet and load into to a 10 mm die
8. Punch under under 0.4 (A) or 0.2 (B) ton pressure, using Specac® manual hydraulic press
9. Collect tablets in a 20 mL vial

TABLE 43
Components and properties of sublingual tablet Formulations 27A, 27B, 28A and 29A

| | Total amounts of | Weights of resin and | # of | | | Disintegration |
|---|---|---|---|---|---|---|
| | resin and excipients | excipients/tablet | tablets | Friability | Hardness | time (s) in 1 mL |
| Formulation | (mg) | (mg) | made | (%) | (N) | PBS |
| 27A | Resin = 102 mg | Resin = 17.0 mg (5.2 mg | 5 | 0.07 | 8.1 | Incomplete |
| (light brown | Mannitol = 305.4 mg | THC; 8.0 mg CBD) | | | | (>6 mins) |
| tablets; | MCC = 92 mg | Mannitol = 50.9 mg | | | | |
| Weight/tablet = | Mg stearate = 4.6 mg | MCC = 15.3 mg | | | | |
| ~96.6 mg) | Kollidon CL ® = 75.5 | Mg stearate = 0.8 mg | | | | |
| | mg | Kollidon CL ® = 12.6 mg | | | | |
| 27B | Resin = 102 mg | Resin = 17.0 mg (5.2 mg | 5 | 0 | 7.9 | Incomplete |
| (light brown | Mannitol = 305.4 mg | THC; 8.0 mg CBD) | | | | (>6 mins) |
| tablets; | MCC = 92 mg | Mannitol = 50.9 mg | | | | |
| Weight/tablet = | Mg stearate = 4.6 mg | MCC = 15.3 mg | | | | |
| ~96.6 mg) | Kollidon CL ® = 75.5 | Mg stearate = 0.8 mg | | | | |
| | mg | Kollidon CL ® = 12.6 mg | | | | |
| 28A | Resin = 102 mg | Resin = 15.6 mg (4.7 mg | 5 | 0.04 | 5.9 | Incomplete |
| (light brown | Mannitol = 350 mg | THC; 7.4 mg CBD) | | | | (>6 mins) |
| tablets; | MCC = 92 mg | Mannitol = 53.6 mg | | | | |
| Weight/tablet = | Mg stearate = 4.6 mg | MCC = 14.1 mg | | | | |
| ~96.5 mg) | Kollidon CL ® = 82.2 | Mg stearate = 0.7 mg | | | | |
| | mg | Kollidon CL ® = 12.6 mg | | | | |
| 29A | Resin = 102 mg | Resin = 16.3 mg (4.9 mg | 5 | 0 | 7.6 | Incomplete |
| (light brown | Mannitol = 305.4 mg | THC; 7.7 mg CBD) | | | | (>6 mins) |
| tablets; | MCC = 92 mg | Mannitol = 48.7 mg | | | | |
| Weight/tablet = | Mg stearate = 4.6 mg | MCC = 14.6 mg | | | | |
| ~96.6 mg) | Kollidon CL ® = 102 | Mg stearate = 0.7 mg | | | | |
| | mg | Kollidon CL ® = 16.3 mg | | | | |

MCC = Microcrystalline cellulose

Procedure for making Formulations 30 and 31:

Formulations 30 and 31 were prepared similarly to Formulations 27-29, except that tablets were punched under 0.1(A) or 0.4 (B) ton pressure.

TABLE 44
Components and properties of sublingual tablet Formulations 30A, 30B, 31A and 31B

| | Total amounts of | Weights of resin and | | | | Disintegration |
|---|---|---|---|---|---|---|
| | resin and excipients | excipients/tablet | # of tablets | Friability | Hardness | time (s) in |
| Formulation | (mg) | (mg) | made | (%) | (N) | 1 mL PBS |
| 30A | Resin = 102 mg | Resin = 9.3 mg (2.8 | 5 | 0.31 | 10.2 | 23 |
| (cream tablets; | Mannitol = 612 mg | mg THC; 4.4 mg | | | | |
| Weight/tablet = | MCC = 183 mg | CBD) | | | | |
| ~96.7 mg) | Mg stearate = 9 mg | Mannitol = 55.9 mg | | | | |
| 30B | Kollidon CL ® = 151 | MCC = 16.7 mg | 5 | 0.79 | 11.0 | 14 |
| (cream tablets; | mg | Mg stearate = 0.8 mg | | | | |
| Weight/tablet = | | Kollidon CL ® = 13.9 | | | | |
| ~96.7 mg) | | mg | | | | |
| 31A | Resin = 102 mg | Resin = 9.3 mg (2.8 | 5 | 0 | 10.0 | Incomplete |
| (cream tablets; | Sorbitol = 612 mg | mg THC; 4.4 mg | | | | (>6 mins) |
| Weight/tablet = | MCC = 183 mg | CBD) | | | | |
| ~96.5 mg) | Mg stearate = 9 mg | Sorbitol = 55.9 mg | | | | |
| 31B | Kollidon CL ® = 151 | MCC = 16.7 mg | 5 | 0 | 10.7 | Incomplete |
| (cream tablets; | mg | Mg stearate = 0.8 mg | | | | (>6 mins) |
| Weight/tablet = | | Kollidon CL ® = 13.9 | | | | |
| ~96.4 mg) | | mg | | | | |

Tables 44 and 45 illustrate experiments where cannabis resin-mannitol powder is developed first, then excipients are added at various concentrations (for example, Kollidon CL®) and/or tablet punching pressure (0.1 or 0.2 or 0.4 ton) to profile for optimal friability, hardness and disintegration times. Higher Kollidon CL® concentrations, and lower tablet punch pressures produced favorable disintegration times and friability properties.

Procedure for making Formulations 32 and 33:

Formulations 32 and 33 were prepared similarly to Formulations 27-29, except:
samples were lyophilized for 65 hrs
tablets were punched under 0.1 (A) or 0.4 (B) ton pressure

TABLE 45
Components and properties of sublingual tablet Formulations 32A, 32B, 33A and 33B

| | | Weights of resin and | # of | | | Disintegration |
|---|---|---|---|---|---|---|
| | Total amounts of resin | excipients/tablet | tablets | Friability | Hardness | time (s) in |
| Formulation # | and excipients (mg) | (mg) | made | (%) | (N) | 1 mL PBS |
| 32A | Resin = 102 mg | Resin = 9.3 mg (2.8 mg | 5 | 0.17 | 7.9 | 10 |
| (cream tablets; | Mannitol = 612 mg | THC; 4.4 mg CBD) | | | | |
| Weight/tablet = | MCC = 183 mg | Mannitol = 55.9 mg | | | | |
| ~96.9 mg) | Mg stearate = 9 mg | MCC = 16.7 mg | | | | |
| *repetition of 51A | Kollidon CL ® = 151 | Mg stearate = 0.8 mg | | | | |
| 32B | mg | Kollidon CL ® = 13.9 | 5 | 0.10 | 7.3 | 15 |
| (cream tablets; | | mg | | | | |
| Weight/tablet = | | | | | | |
| ~96.8 mg) | | | | | | |
| *repetition of 51B | | | | | | |
| 33A | Resin = 102 mg | Resin = 11.6 mg (3.5 | 4 | 0.07 | 6.11 | 16 |
| (cream tablets; | Mannitol = 400 mg | mg THC; 5.5 mg CBD) | | | | |
| Weight/tablet = | MCC = 183 mg | Mannitol = 45.7 mg | | | | |
| ~96.5 mg) | Mg stearate = 9 mg | MCC = 20.9 mg | | | | |
| 33B | Kollidon CL ® = 151 | Mg stearate = 1.0 mg | 4 | 0.07 | 5.26 | 16 |
| (cream tablets; | mg | Kollidon CL ® = 17.2 | | | | |
| Weight/tablet = | | mg | | | | |
| ~96.5 mg) | | | | | | |

Procedure for making Formulations 34 and 35:

The procedures for making Formulations 32B and 33B (0.4 ton pressure only) were repeated on a larger scale to afford >20 tablets.

TABLE 46
Components and properties of sublingual tablet Formulations 34 and 35

| | | | | | | Disintegration | |
|---|---|---|---|---|---|---|---|
| | Total amounts of | Weights of resin and | # of | | | time (s) in 1 mL | |
| | resin and excipients | excipients/tablet | tablets | Friability | Hardness | | Artificial |
| Formulation | (mg) | (mg) | made | (%) | (N) | PBS | saliva |
| 34 | Resin = 306 mg | Resin = 11.6 mg (5.2 mg | 25 | 0 | 4.9 | 71 | 167 |
| (beige tablets; | Mannitol = 1200 mg | THC; 8.3 mg CBD) | | | | | |
| Weight/tablet = | MCC = 549 mg | Mannitol = 45.7 mg | | | | | |
| ~96.5 mg) | Mg stearate = 27 mg | MCC = 20.9 mg | | | | | |
| *repetition of | Kollidon CL ® = 453 | Mg stearate = 1.0 mg | | | | | |
| 33B | mg | Kollidon CL ® = 17.2 mg | | | | | |
| 35 | Resin = 306 mg | Resin = 9.3 mg (4.1 mg | 32 | 0 | 6.1 | 17 | 60 |
| (cream tablets; | Mannitol = 1836 mg | THC; 6.6 mg CBD) | | | | | |
| Weight/tablet = | MCC = 549 mg | Mannitol = 55.9 mg | | | | | |
| ~96.5 mg) | Mg stearate = 27 mg | MCC = 16.7 mg | | | | | |
| *repetition of | Kollidon CL ® = 453 | Mg stearate = 0.8 mg | | | | | |
| 32B | mg | Kollidon CL ® = 13.9 mg | | | | | |

Procedure for making Formulations 36 and 37:

Formulations 36 and 37 were prepared similarly to 27-29, except:
that samples were lyophilized for 65 hrs
˜200 mg per tablet
tablets were punched under 0.1(A) or 0.4 (B) ton pressure

TABLE 47
Components and properties of sublingual tablet Formulations 36A, 36B, 37A and 37B

| | Total amounts of | Weights of resin and | # of | | | Disintegration |
|---|---|---|---|---|---|---|
| | resin and excipients | excipients/tablet | tablets | Friability | Hardness | time (s) in 1 mL |
| Formulation | (mg) | (mg) | made | (%) | (N) | artificial saliva |
| 36A | Resin = 195 mg | Resin = 24.1 mg (8.7 | 4 | 0.12 | 18.6 | 78 |
| (beige tablets; | Mannitol = 765 mg | mg THC; 13.8 mg | | | | |
| Weight/tablet = | MCC = 350 mg | CBD) | | | | |
| ~200.4 mg) | Mg stearate = 17 mg | Mannitol = 94.7 mg | | | | |
| 36B | Kollidon CL ® = 289 | MCC = 43.3 mg | 4 | 0.25 | 17.1 | 66 |
| (beige tablets; | mg | Mg stearate = 2.1 mg | | | | |
| Weight/tablet = | | Kollidon CL ® = 35.8 | | | | |
| ~199.8 mg) | | mg | | | | |
| 37A | Resin = 204 mg | Resin = 19.3 mg (6.5 | 5 | 0.17 | 26.8 | 90 |
| (cream tablets; | Mannitol = 1224 mg | mg THC; 10.3 mg | | | | |
| Weight/tablet = | MCC = 366 mg | CBD) | | | | |
| ~200.0 mg) | Mg stearate = 18 mg | Mannitol = 115.8 mg | | | | |
| 37B | Kollidon CLO = 302 | MCC = 34.6 mg | 5 | 0.13 | 21.7 | 98 |
| (cream tablets; | mg | Mg stearate = 1.7 mg | | | | |
| Weight/tablet = | | Kollidon CL ® = 13.9 | | | | |
| ~199.7 mg) | | mg | | | | |

Procedure for making Formulations 38 and 39:
1. Formulations 38 and 39 were prepared similarly to Formulations 36 and 37, except that the tablet weight in Formulations 38 and 39 is about 200 mg/tablet, and in Formulations 36 and 37, it is about 100 mg/tablet. Removed 0.51 mL of 200 mg/mL of resin solution, placed in vial and diluted to 1 mL with ethanol (102 mg of resin)
2. A solution of mannitol (306 mg) in 4 mL water was added
3. Added 3 more mL of water
4. Lyophilized for 65 hrs
5. Added 306 mg amount of mannitol, in addition other excipients in quantities as shown in Table 48
6. Triturated in a porcelain mortar with a pestle until having a homogeneous solid solution
7. Weighed ˜100 mg of the mixture for each tablet and load into a 10 mm die
8. Punched under 0.1 (A) or 0.4 (B) ton pressure, using Specac® manual hydraulic press
9. Collected tablets in a 20 mL vial

TABLE 48
Components and properties of sublingual tablet Formulations 38A, 38B, 39A and 39B

| | Total amounts of | Weights of resin and | # of | | | Disintegration |
|---|---|---|---|---|---|---|
| | resin and | excipients/tablet | tablets | Friability | Hardness | time (s) in 1 mL |
| Formulation | excipients (mg) | (mg) | made | (%) | (N) | artificial saliva |
| 38A | Resin = 102 mg | Resin = 9.3 mg (3.1 | 4 | 0.35 | 7.8 | 153 |
| (beige tablets; | Mannitol = 612 mg | mg THC; 5.0 mg | | | | |
| Weight/tablet = | MCC = 183 mg | CBD) | | | | |
| ~99.9 mg) | Mg stearate = 9 mg | Mannitol = 55.9 mg | | | | |
| 38B | Kollidon CL ® = | MCC = 16.7 mg | 4 | 0.20 | 7.0 | 130 |
| (beige tablets; | 151 mg | Mg stearate = 0.8 mg | | | | |
| Weight/tablet = | | Kollidon CL ® = 13.9 | | | | |
| ~100.1 mg) | | mg | | | | |
| 39A | Resin = 102 mg | Resin = 11.6 mg (3.9 | 4 | 0.05 | 7.8 | 166 |
| (beige tablets; | Mannitol = 400 mg | mg THC; 6.2 mg | | | | |
| Weight/tablet = | MCC = 183 mg | CBD) | | | | |
| ~100.1 mg) | Mg stearate = 9 mg | Mannitol = 45.7 mg | | | | |
| 39B | Kollidon CL ® = | MCC = 20.9 mg | 5 | 0.10 | 6.9 | 167 |
| (beige tablets; | 151 mg | Mg stearate = 1.0 mg | | | | |
| Weight/tablet = | | Kollidon CL ® = 17.2 | | | | |
| ~99.1 mg) | | mg | | | | |

Formulations 40 and 41 were made using the same procedure as Formulations 38 and 39.

TABLE 49
Components and properties of sublingual tablet Formulations 40A, 40B, 41A and 41B

| | Total amounts of | Weights of resin and | # of | | | Disintegration |
|---|---|---|---|---|---|---|
| | resin and excipients | excipients/tablet | tablets | Friability | Hardness | time (s) in 1 mL |
| Formulation | (mg) | (mg) | made | (%) | (N) | artificial saliva |
| 40A | Resin =204 mg | Resin = 24.1 mg (3.9 | 4 | 0 | 16.8 | 170 |
| (beige tablets; | Mannitol = 800 mg | mg THC; 6.2 mg CBD) | | | | |
| Weight/tablet = | MCC = 366 mg | Mannitol = 95.7 mg | | | | |
| ~199.7 mg) | Mg stearate = 18 mg | MCC = 43.3 mg | | | | |
| 40B | Kollidon CL ® = 302 | Mg stearate = 2.1 mg | 4 | 0.02 | 12.3 | 158 |
| (beige tablets; | mg | Kollidon CL ® = 35.7 | | | | |
| Weight/tablet = | | mg | | | | |
| ~200.2 mg) | | | | | | |
| 41A | Resin = 204 mg | Resin = 19.3 mg (6.5 | 5 | 0.05 | 8.5 | 124 |
| (beige tablets; | Mannitol = 1224 mg | mg THC; 10.3 mg | | | | |
| Weight/tablet = | MCC = 366 mg | CBD) | | | | |
| ~199.9 mg) | Mg stearate = 18 mg | Mannitol = 115.8 mg | | | | |
| 41B | Kollidon CL ® = 302 | MCC = 34.6 mg | 5 | 0.0 | 7.5 | 107 |
| (beige tablets; | mg | Mg stearate = 1.7 mg | | | | |
| Weight/tablet = | | Kollidon CL ® = 13.9 | | | | |
| ~200.0 mg) | | mg | | | | |

Procedure for making Formulation 42:
1. Removed 0.51 mL of 200 mg/mL solution of resin, placed in vial and diluted to 1 mL with ethanol (102 mg of resin)
2. A solution of mannitol (400 mg) in 4 mL water was added
3. Added 3 more mL of water
4. Lyophilized all vials for 67 hrs
5. Added remaining amount of mannitol (212 mg) as well as other excipients in quantities as shown in Table 50
6. Triturated in a porcelain mortar with a pestle until having a homogeneous solid solution
7. Weighed ˜36 mg of the mixture for each tablet and load into a 3 mm die
8. Punched under 0.1 ton pressure, using Specac® manual hydraulic press
9. Collected tablets in a 20 mL vial

TABLE 50
Components and properties of sublingual tablet Formulation 42

| | | | | | | Disintegration time | |
|---|---|---|---|---|---|---|---|
| | Total amounts of | | # of | | | (s) in 1 mL | |
| | resin and excipients | Weights of resin and | tablets | Friability | Hardness | | Artificial |
| Formulation | (mg) | excipients/tablet (mg) | made | (%) | (N) | PBS | saliva |
| 42 | Resin = 102 mg | Resin = 3.5 mg (1.1 mg | 28 | 0.6 | 60.4 | 26.5 | 43.5 |
| (beige tablets; | Mannitol = 612 mg | THC; 1.5 mg CBD) | | | | | |
| Weight/tablet = | MCC = 183 mg | Mannitol = 20.8 mg | | | | | |
| ~35.5 mg) | Mg stearate = 9 mg | MCC = 6.2 mg | | | | | |
| | Kollidon CL ® = 151 | Mg stearate = 0.3 mg | | | | | |
| | mg | Kollidon CL ® = 5.1 mg | | | | | |

### Experiment 19: Determination of Stability of the Sublingual Tablet

Strain 1 resin used was found to contain 31.7% THC and 46.1% CBD. Procedure for making Formulation 43:
1. Resin (0.2965 g) was dissolved in ethanol (2.965 mL) to give a 100 mg/mL solution
2. Divided ethanolic solution into three portions, then made up volume to 1 mL with ethanol (98.8 mg of resin per vial)
3. A solution of mannitol (593 mg) in 4 mL water was added to each vial
4. Added 3 more mL of water
5. Lyophilized all vials for 42 hrs
6. Combined vials and added other excipients in quantities as shown in Table 51
7. Triturated in a porcelain mortar with a pestle until having a homogeneous solid solution
8. Weighed ˜96.5 mg of the mixture for each tablet and load into a 10 mm die
9. Punched under 0.4 ton pressure, using Specac® manual hydraulic press
10. Collected tablets in a 20 mL vial
11. The tablets were placed in the humidity chamber at 37° C. and 40% humidity on day 2 and the quality of the tablets determined at specific time periods

TABLE 51
Components of sublingual tablet Formulation 43

| | | | # of |
|---|---|---|---|
| | Total amounts of resin | Weights of resin and | tablets |
| Formulation | and excipients (mg) | excipients/tablet (mg) | made |
| 43 | Resin = 296.5 mg | Resin = 9.3 mg (2.9 mg THC; 4.2 mg | 31 |
| (beige tablets; | Mannitol = 1779 mg | CBD) | |
| weight/tablet = ~96.4 mg) | MCC = 534 mg | Mannitol = 55.8 mg | |
| | Mg stearate = 27 mg | MCC = 16.7 mg | |
| | Kollidon CL ® = 439 mg | Mg stearate = 0.8 mg | |
| | | Kollidon CL ® = 13.8 mg | |

TABLE 52
Properties of sublingual tablet Formulation 43

| | | | | Disintegration | |
|---|---|---|---|---|---|
| | | | | time (s) in 1 mL | |
| | | Friability | Hardness | | Artificial |
| Formulation | Days | (%) | (N) | PBS | saliva |
| 43 | 1a | 0.78 | 7.8 ± 0.3 | 20.5 ± 0.7 | 64.0 ± 1.4 |
| (CDS-ML-II- | 8a | 0.40 | 13.7 ± 0.7 | 17.5 ± 2.1 | 54.0 ± 12.7 |
| 66-1) | 23a | 0.05 | 17.1 ± 1.7 | 20.5 ± 2.1 | 87.0 ± 4.2 |

The same tablets were used for friability first then hardness.
aN = 2 tablets

This study is meant to observe the changes to the hardness, friability and disintegration times of the tablets over the course of time. A favorable profile will not alter disintegration times significantly, exceeding 120 seconds or so, for example in PBS. While there is no set rule to accept or reject a particular outcome, ideally one would like to have disintegration within 1-2 min (60-120 sec), even after storing the product for several days.

### Experiment 20: Further Studies of Sublingual RDT Formulations

Formulation 44 comprised decarboxylated cannabis resin from strain 5, containing 36.0% THC and 60.0% CBD, and was made by the following procedure:
1. Resin (0.1025 g) was dissolved in ethanol (1.02 mL) to give a 100 mg/mL solution
2. A solution of mannitol (612 mg) in 4 mL water was added to each vial
3. See steps 4-8 as described above in Formulation 43
4. Punch under 0.1 (A) or 0.4 (B) ton pressure, using Specac® manual hydraulic press

TABLE 53
Components and properties of sublingual tablet Formulations 44A
and 44B; physical properties measured on day 0

| | | Weights of resin | | | | Disintegration | |
|---|---|---|---|---|---|---|---|
| | Total amounts | and | # of | | | time(s) in 1 mL^{b} | |
| | of resin and | excipients/tablet | tablets | Friability^{a} | Hardness | | Artificial |
| Formulation | excipients (mg) | (mg) | made | (%) | (N) | PBS | saliva |
| 44A | Resin = 102.5 mg | Resin = 9.4 mg | 5 | 0.47 | 11.7 ± 5.9 | 40 | >6 mins |
| (beige | Mannitol = 612 | (3.4 mg THC; 5.6 | | | | | |
| tablets; | mg | mg CBD) | | | | | |
| Weight/table | MCC = 183 mg | Mannitol = 55.9 | | | | | |
| t = ~96.6 mg) | Mg stearate = 9 | mg | | | | | |
| | mg | MCC = 16.7 mg | | | | | |
| | Kollidon CL ® = | Mg stearate = 0.8 | | | | | |
| | 151 mg | mg | | | | | |
| | | Kollidon CL ® = | | | | | |
| | | 13.8 mg | | | | | |
| 44B | | Resin = 3.3 mg | 5 | 0.16 | 10.0 ± 2.9 | 45 | >6 mins |
| (beige | | (3.4 mg THC; 5.6 | | | | | |
| tablets; | | mg CBD) | | | | | |
| Weight/table | | Mannitol = 55.8 | | | | | |
| t = ~96.4 mg) | | mg | | | | | |
| | | MCC = 16.7 mg | | | | | |
| | | Mg stearate = 0.8 | | | | | |
| | | mg | | | | | |
| | | Kollidon CL ® = | | | | | |
| | | 13.8 mg | | | | | |

^{a}The same tablets were used for friability first then hardness; N = 2 tablets
^{b}N = 1 tablet

Formulation 45 comprised decarboxylated cannabis resin from strain 3, containing 49.8% THC and <1% CBD, and was made by the following procedure:
1. Resin (0.1974 g) was dissolved in ethanol (1.974 mL) to give a 100 mg/mL solution
2. Divided ethanolic solution into two portions, then made up volume to 1 mL with ethanol (98.7 mg of resin per vial)
3. A solution of mannitol (592 mg) in 4 mL water was added to each vial
4. See steps 4-8 as described above in Formulation 43
5. Punch under 0.1 (A) or 0.4 (B) ton pressure, using Specac® manual hydraulic press

TABLE 54
Components and properties of sublingual tablet Formulations 45A
and 45B; physical properties measured on day 1

| | | Weights of resin | | | | Disintegration | |
|---|---|---|---|---|---|---|---|
| | Total amounts | and | # of | | | time(s) in 1 mL^{b} | |
| | of resin and | excipients/tablet | tablets | Friability^{a} | Hardness | | Artificial |
| Formulation | excipients (mg) | (mg) | made | (%) | (N) | PBS | saliva |
| 45A | Resin = 197.4 mg | Resin = 11.1 mg | 8 | 0.26 | 9.8 ± 0.6 | 33 | 224 |
| (cream tablets; | Mannitol = 1184 | (5.5 mg THC) | | | | | |
| Weight/tablet = | mg | Mannitol = 66.4 | | | | | |
| ~96.1 mg) | MCC = 178 mg | mg | | | | | |
| 45B | Mg stearate = 9 mg | MCC = 10.0 mg | 8 | 0.36 | 9.7 ± 0.4 | 31 | 191 |
| (cream tablets; | Kollidon CL ® = | Mg stearate = 0.5 | | | | | |
| Weight/tablet = | 146 mg | mg | | | | | |
| ~96.2 mg) | | Kollidon CL ® = | | | | | |
| | | 8.2 mg | | | | | |

^{a}The same tablets were used for friability first then hardness; N = 2 tablets
^{b}N = 1 tablet

Formulation 46 comprised decarboxylated cannabis resin from strain 2, containing 1.9% THC and 37.6% CBD, and was made by the following procedure:
1. Resin (0.1552 g) was dissolved in ethanol (1.552 mL) to give a 100 mg/mL solution
2. Divided ethanolic solution into two portions, then made up volume to 1 mL with ethanol (77.6 mg of resin per vial)
3. A solution of mannitol (466 mg) in 4 mL water was added to each vial
4. See steps 4-8 as described above in Formulation 43
5. Punch under 0.1 (A) or 0.4 (B) ton pressure, using Specac® manual hydraulic press

TABLE 55
Components and properties of sublingual tablet Formulations 46A
and 46B; physical properties measured on day 1

| | | Weights of resin | | | | Disintegration | |
|---|---|---|---|---|---|---|---|
| | Total amounts | and | # of | | | time(s) in 1 mL^{b} | |
| | of resin and | excipients/tablet | tablets | Friability^{a} | Hardness | | Artificial |
| Formulation | excipients (mg) | (mg) | made | (%) | (N) | PBS | saliva |
| 46A | Resin = 155.2 mg | Resin = 11.1 mg | 6 | 0.05 | 5.5 ± 0.4 | 30 | 362 |
| (cream tablets; | Mannitol = 931 mg | (0.2 mg THC, | | | | | |
| Weight/tablet = | MCC = 140 mg | 4.2 mg CBD) | | | | | |
| ~96.3 mg) | Mg stearate = 7 mg | Mannitol = 66.5 | | | | | |
| 46B | Kollidon CL ® = | mg | 6 | 0.05 | 8.5 ± 0.7 | 29 | 361 |
| (cream tablets; | 115 mg | MCC = 10.0 mg | | | | | |
| Weight/tablet = | | Mg stearate = 0.5 | | | | | |
| ~96.6 mg) | | mg | | | | | |
| | | Kollidon CL ® = | | | | | |
| | | 8.2 mg | | | | | |

^{a}The same tablets were used for friability first then hardness; N = 2 tablets
^{b}N = 1 tablet

These experiments (Formulations 44-46) suggest that decarboxylated cannabis resins containing various ratios of THC and CBD in them may tend to exhibit similar disintegration behavior (under 50 sec), and low friability (<1%), although hardness differed noticeably. A person of skill in the art would be able to make minor adjustments to the disclosed excipients to accommodate the properties of such tablet dosage forms to acceptable levels.

Formulation 47 was made according to the procedure used for making Formulation 42. Cannabis resin constituents (THC and CBD) are 1.1 mg/1.5 mg in Formulation 42, and 1.3 mg/1.4 mg in Formulation 47, and the total THC+CBD content is 2.6 mg and 2.7 mg, respectively.

TABLE 56
Components and properties of sublingual tablet Formulations 47;
physical properties measured on day 1

| | | Weights of resin | | | | Disintegration | |
|---|---|---|---|---|---|---|---|
| | Total amounts | and | # of | | | time(s) in 1 mL | |
| | of resin and | excipients/tablet | tablets | Friability | Hardness | | Artificial |
| Formulation | excipients (mg) | (mg) | made | (%) | (N) | PBS | saliva |
| 47 | Resin = 102 mg | Resin = 3.4 mg | 28 | 0.14 | 112.0 | 8 | 46.5 |
| (beige tablets; | Mannitol = 612 mg | (equiv. 1.3 mg THC; | | | | | |
| Weight/tablet = | MCC = 183 mg | 1.4 mg CBD) | | | | | |
| ~35.7 mg) | Mg stearate = 9 mg | Mannitol = 20.7 mg | | | | | |
| | Kollidon CL ® = | MCC = 6.2 mg | | | | | |
| | 151 mg | Mg stearate = 0.3 mg | | | | | |
| | | Kollidon CL ® = | | | | | |
| | | 5.1 mg | | | | | |

Formulation 48 was made according to the procedure used for making Formulation 44.

TABLE 57
Components and properties of sublingual tablet Formulations 48A
and 48B; physical properties measured on day 1

| | | Weights of resin | | | | Disintegration | |
|---|---|---|---|---|---|---|---|
| | Total amounts | and | # of | | | time(s) in 1 mL | |
| | of resin and | excipients/tablet | tablets | Friability | Hardness | | Artificial |
| Formulation | excipients (mg) | (mg) | made | (%) | (N) | PBS | saliva |
| 48A | Resin = 102 mg | Resin = 9.2 mg | 5 | 0.10 | 5.1 | 16 | 2185 |
| (cream tablets; | Mannitol = 795 mg | (2.6 mg THC; | | | | | |
| Weight/tablet = | Mg stearate = 9 mg | 3.5 mg CBD) | | | | | |
| ~96.6 mg) | Kollidon CL ® = | Mannitol = 72.7 mg | | | | | |
| | 151 mg | Mg stearate = 0.8 mg | | | | | |
| | | Kollidon CL ® = | | | | | |
| | | 13.8 mg | | | | | |
| 48B | | Resin = 9.3 mg | 5 | 0.10 | 6.8 | 27 | 298 |
| (cream tablets; | | (2.6 mg THC; | | | | | |
| Weight/tablet = | | 3.5 mg CBD) | | | | | |
| ~96.4 mg) | | Mannitol = 72.5 mg | | | | | |
| | | Mg stearate = 0.8 mg | | | | | |
| | | Kollidon CL ® = | | | | | |
| | | 13.8 mg | | | | | |

Formulations 49-51 were made according to the procedure used for making Formulation 44.

TABLE 58
Components and properties of sublingual tablet Formulations 49A, 49B, 50A,
50B, 51A and 51B; physical properties measured on day 1

| | | Weights of resin | | | | Disintegration | |
|---|---|---|---|---|---|---|---|
| | Total amounts | and | # of | | | time(s) in 1 mL | |
| | of resin and | excipients/tablet | tablets | Friability | Hardness | | Artificial |
| Formulation | excipients (mg) | (mg) | made | (%) | (N) | PBS | saliva |
| 49A | Resin = 102 mg | Resin = 9.3 mg | 5 | 0.10 | 8.2 | 30 | 203 |
| (cream tablets; | Mannitol = 612 mg | (3.4 mg THC; | | | | | |
| Weight/tablet = | MCC = 183 mg | 4.0 mg CBD) | | | | | |
| ~96.4 mg) | Mg stearate = 9 mg | Mannitol = 55.8 mg | | | | | |
| 49B | *CCS = 151 mg | MCC = 16.7 mg | 5 | 0.10 | 9.6 | 18 | 249 |
| (cream tablets; | | Mg stearate = 0.8 mg | | | | | |
| Weight/tablet = | | CCS = 13.8 mg | | | | | |
| ~96.4 mg) | | | | | | | |
| 50A | Resin = 102 mg | Resin = 9.3 mg | 5 | 0 | 6.6 | 50 | >6 mins |
| (cream tablets; | Mannitol = 612 mg | (3.4 mg THC; | | | | | |
| Weight/tablet = | MCC = 183 mg | 4.0 mg CBD) | | | | | |
| ~96.3 mg) | Mg stearate = 9 mg | Mannitol = 55.8 mg | | | | | |
| 50B | #SSG = 151 mg | MCC = 16.7 mg | 5 | 0.21 | 9.0 | 112 | >6 mins |
| (cream tablets; | | Mg stearate = 0.8 mg | | | | | |
| Weight/tablet = | | SSG = 13.8 mg | | | | | |
| ~96.2 mg) | | | | | | | |
| 51A | Resin = 102 mg | Resin = 9.3 mg | 5 | 0.10 | 8.0 | 26 | >6 mins |
| (cream tablets; | Mannitol = 612 mg | (3.4 mg THC; | | | | | |
| Weight/tablet = | MCC = 183 mg | 4.0 mg CBD) | | | | | |
| ~96.4 mg) | Mg stearate = 9 mg | Mannitol = 55.8 mg | | | | | |
| 51B | Polyplasdone = | MCC = 16.7 mg | 5 | 0.16 | 9.1 | 26 | >6 mins |
| (cream tablets; | 151 mg | Mg stearate = 0.8 mg | | | | | |
| Weight/tablet = | | Polyplasdone = | | | | | |
| ~96.4 mg) | | 13.8 mg | | | | | |

*CCS = Croscarmellose sodium
#SSG = Sodium starch glycolate

Formulations 52 and 53 were made according to the procedure used for making Formulation 44.

TABLE 59
Components and properties of sublingual tablet Formulations 52A, 52B, 53A
and 53B; physical properties measured on day 1

| | | Weights of resin | | | | Disintegration | |
|---|---|---|---|---|---|---|---|
| | Total amounts | and | # of | | | time(s) in 1 mL | |
| | of resin and | excipients/tablet | tablets | Friability | Hardness | | Artificial |
| Formulation | excipients (mg) | (mg) | made | (%) | (N) | PBS | saliva |
| 52A | Resin = 102 mg | Resin = 9.3 mg | 5 | 0.05 | 8.8 | >6 mins | >6 mins |
| (cream tablets; | Mannitol = 612 mg | (3.0 mg THC; | | | | | |
| Weight/tablet = | MCC = 183 mg | 3.6 mg CBD) | | | | | |
| ~96.4 mg) | Mg stearate = 9 mg | Mannitol = 55.8 mg | | | | | |
| 52B | Klucel ™ (HPC) = | MCC = 16.7 mg | 5 | 0.05 | 9.8 | >6 mins | >6 mins |
| (cream tablets; | 151 mg | Mg stearate = 0.8 mg | | | | | |
| Weight/tablet = | | Klucel ™ (HPC) = | | | | | |
| ~96.4 mg) | | 13.8 mg | | | | | |
| 53A | Resin = 102 mg | Resin = 9.3 mg | 5 | 0 | 7.8 | 59 | >6 mins |
| (cream tablets; | Mannitol = 612 mg | (3.0 mg THC; | | | | | |
| Weight/tablet = | MCC = 183 mg | 3.6 mg CBD) | | | | | |
| ~96.2 mg) | Mg stearate = 9 mg | Mannitol = 55.7 mg | | | | | |
| | L-HPC = 151 mg | MCC = 16.7 mg | | | | | |
| | | Mg stearate = 0.8 mg | | | | | |
| | | L-HPC = 13.7 mg | | | | | |
| 53B | | Resin = 9.6 mg | 4 | 0 | 6.6 | 73 | >6 mins |
| (cream tablets; | | (3.1 mg THC; | | | | | |
| Weight/tablet = | | 3.7 mg CBD) | | | | | |
| ~99.5 mg) | | Mannitol = 57.6 mg | | | | | |
| | | MCC = 16.7 mg | | | | | |
| | | Mg stearate = 0.8 mg | | | | | |
| | | L-HPC = 14.2 mg | | | | | |

*HPC = hydroxypropyl cellulose
*L-HPC = low-substituted hydroxypropyl cellulose

Formulation 54 was made according to the procedure used for making Formulation 44.

TABLE 60
Components and properties of sublingual tablet Formulations 54A
and 54B; physical properties measured on day 1

| | | Weights of resin | | | | Disintegration | |
|---|---|---|---|---|---|---|---|
| | Total amounts | and | # of | | | time(s) in 1 mL | |
| | of resin and | excipients/tablet | tablets | Friability | Hardness | | Artificial |
| Formulation | excipients (mg) | (mg) | made | (%) | (N) | PBS | saliva |
| 54A | Resin (Strain 1) = | Resin = 9.8 mg | 9 | 2.4 | 3.7 | 11 | 340 |
| (cream tablets; | 197.2 mg | (3.0 mg THC; | | | | | |
| Weight/tablet = | Mannitol = 1183 mg | 4.1 mg CBD) | | | | | |
| ~101.7 mg) | MCC = 177 mg | Mannitol = 58.8 mg | | | | | |
| | Mg stearate = 18 mg | MCC = 8.8 mg | | | | | |
| | Pol 407 = 177 mg | Mg stearate = 0.9 mg | | | | | |
| | Kollidon CL ® = | Pol 407 = 8.8 mg | | | | | |
| | 292 mg | Kollidon CL ® = | | | | | |
| | | 14.5 mg | | | | | |
| 54B | | Resin = 9.4 mg | 9 | 0 | Failed | 12 | 235 |
| (cream tablets; | | (2.9 mg THC; | | | | | |
| Weight/tablet = | | 3.9 mg CBD) | | | | | |
| ~97.3 mg) | | Mannitol = 56.3 mg | | | | | |
| | | MCC = 8.4 mg | | | | | |
| | | Mg stearate = 0.8 mg | | | | | |
| | | Pol 407 = | | | | | |
| | | Kollidon CL ® = | | | | | |
| | | 13.9 mg | | | | | |

### Experiment 21: A Trial to Substitute Mannitol with Sucrose

Procedure for making Formulation 55:
1. Weighed resin into 2 vials and dissolved in 1 mL EtOH each
2. A solution of sucrose in 4 mL water was added to one vial and another solution of xylitol in 4 mL water was added to the other vial
3. Added 3 more mL of water to each vial
4. Lyophilized all vials for 43 hrs Note: After lyophilization, it was noted that vial with xylitol was a viscous liquid, therefore nothing further was done
5. Added other excipients to sucrose formulation in quantities as shown in table
6. Triturated in a porcelain mortar with a pestle until having a homogeneous solid solution
7. Weighed mixture for each tablet and load into a 10 mm die
8. Punched under 0.1 (A) or 0.4 (B) ton pressure, using Specac® manual hydraulic press
9. Collected tablets in 20 mL vials

TABLE 61
Components and properties of sublingual tablet Formulations 55A
and 55B; physical properties measured on day 1

| | | Weights of resin | | | | Disintegration | |
|---|---|---|---|---|---|---|---|
| | Total amounts | and | # of | | | time(s) in 1 mL | |
| | of resin and | excipients/tablet | tablets | Friability | Hardness | | Artificial |
| Formulation # | excipients (mg) | (mg) | made | (%) | (N) | PBS | saliva |
| 55A | Resin = 101.3 mg | Resin = 9.4 mg | 5 | 0.67 | 4.5 | >6 mins | >6 mins |
| (brown tablets; | Sucrose = 608 mg | (3.5 mg THC; | | | (4.9, 4.1) | | |
| Weight/tablet = | MCC = 182 mg | 4.0 mg CBD) | | | | | |
| ~97.2 mg) | Mg stearate = 9 mg | Sucrose = 56.2 mg | | | | | |
| | Kollidon CL ® = | MCC = 16.9 mg | | | | | |
| | 150 mg | Mg stearate = 0.8 mg | | | | | |
| | | Kollidon CL ® = | | | | | |
| | | 13.9 mg | | | | | |
| 55B | | Resin = 9.3 mg | 5 | 0.46 | 8.4 | >6 mins | >6 mins |
| (brown tablets; | | (3.5 mg THC; | | | (8.4, 8.3) | | |
| Weight/tablet = | | 4.0 mg CBD) | | | | | |
| ~96.9 mg) | | Sucrose = 56.1 mg | | | | | |
| | | MCC = 16.8 mg | | | | | |
| | | Mg stearate = 0.8 mg | | | | | |
| | | Kollidon CL ® = | | | | | |
| | | 13.8 mg | | | | | |

### Experiment 22: Incorporation of an Effervescent Mixture into the Sublingual Tablet Formulations

Formulation 56-59: (A trial to incorporate an effervescent mixture into the RDT formulation along with head-to-head comparison between cannabis Strain 1 and Strain 4 resins in the same formulation).

Formulations 56-59 were made according to the procedure used for making Formulation 44.

TABLE 62
Components and properties of sublingual tablet Formulations 56A, 56B, 57A,
57B, 58A, 58B, 59A and 59B; physical properties measured on day 1

| | | Weights of resin | | | | Disintegration | |
|---|---|---|---|---|---|---|---|
| | Total amounts | and | # of | | | time(s) in 1 mL | |
| | of resin and | excipients/tablet | tablets | Friability | Hardness | | Artificial |
| Formulation | excipients (mg) | (mg) | made | (%) | (N) | PBS | saliva |
| 56A | Resin (Strain 1) = | Resin = 9.4 mg | 5 | 0 | 4.3 | 37 | >6 mins |
| (cream tablets; | 102 mg | (3.0 mg THC; | | | | | |
| Weight/tablet = | Mannitol = 612 mg | 3.6 mg CBD) | | | | | |
| ~106.0 mg) | Mg stearate = 9 mg | Mannitol = 56.4 mg | | | | | |
| | Kollidon CL ® = | Mg stearate = 0.8 mg | | | | | |
| | 334 mg | Kollidon CL ® = | | | | | |
| | Citric acid = 40 mg | 30.8 mg | | | | | |
| | NaHCO3 = 53 mg | Citric acid = 3.7 mg | | | | | |
| | | NaHCO3 = 4.9 mg | | | | | |
| 56B | | Resin = 9.4 mg | 5 | 0 | 6.1 | 48 | >6 mins |
| (cream tablets; | | (3.0 mg THC; | | | | | |
| Weight/tablet = | | 3.6 mg CBD) | | | | | |
| ~105.8 mg) | | Mannitol = 56.3 mg | | | | | |
| | | Mg stearate = 0.8 mg | | | | | |
| | | Kollidon CL ® = | | | | | |
| | | 30.7 mg | | | | | |
| | | Citric acid = 3.7 mg | | | | | |
| | | NaHCO3 = 4.9 mg | | | | | |
| 57A | Resin (Strain 1) = | Resin = 9.2 mg | 6 | 0.05 | 3.6 | 33 | >6 mins |
| (cream tablets; | 109.1 mg | (3.5 mg THC; | | | | | |
| Weight/tablet = | Mannitol = 654 mg | 3.9 mg CBD) | | | | | |
| ~100.0 mg) | MCC = 196 mg | Mannitol = 55.4 mg | | | | | |
| | Mg stearate = 10 mg | MCC = 16.6 mg | | | | | |
| | Kollidon CL ® = | Mg stearate = 0.8 mg | | | | | |
| | 161 mg | Kollidon CL ® = | | | | | |
| | Citric acid = 21 mg | 13.7 mg | | | | | |
| | NaHCO3 = 28 mg | Citric acid = 1.8 mg | | | | | |
| | | NaHCO3 = 2.4 mg | | | | | |
| 57B | | Resin = 9.2 mg | 5 | 0.15 | 4.36 | 36 | >6 mins |
| (cream tablets; | | (3.5 mg THC; | | | | | |
| Weight/tablet = | | 3.9 mg CBD) | | | | | |
| ~99.7 mg) | | Mannitol = 55.3 mg | | | | | |
| | | MCC = 16.6 mg | | | | | |
| | | Mg stearate = 0.8 mg | | | | | |
| | | Kollidon CL ® = | | | | | |
| | | 13.6 mg | | | | | |
| | | Citric acid = 1.8 mg | | | | | |
| | | NaHCO3 = 2.4 mg | | | | | |
| 58A | Resin (Strain 4) = | Resin = 9.4 mg | 5 | 0.4 | 3.0 | 35 | |
| (cream tablets; | 96.2 mg | (THC and CBD = | | | | | |
| Weight/tablet = | Mannitol = 577 mg | TBD) | | | | | |
| ~103.1 mg) | Mg stearate = 9 mg | Mannitol = 56.3 mg | | | | | |
| | Kollidon CL ® = | Mg stearate = 0.8 mg | | | | | |
| | 275 mg | Kollidon CL ® = | | | | | |
| | Citric acid = 36 mg | 26.8 mg | | | | | |
| | NaHCO3 = 48 mg | Citric acid = 3.5 mg | | | | | |
| | | NaHCO3 = 4.6 mg | | | | | |
| 58B | | Resin = 9.5 mg | 5 | 0.4 | 4.9 | 40 | |
| (cream tablets; | | (THC and CBD = | | | | | |
| Weight/tablet = | | TBD) | | | | | |
| ~101.5 mg) | | Mannitol = 57.2 mg | | | | | |
| | | Mg stearate = 0.9 mg | | | | | |
| | | Kollidon CL ® = | | | | | |
| | | 27.3 mg | | | | | |
| | | Citric acid = 3.5 mg | | | | | |
| | | NaHCO3 = 4.7 mg | | | | | |
| 59A | Resin (Strain 4) = | Resin = 9.4 mg | 5 | 0.5 | 2.3 | 22 | |
| (cream tablets; | 96.2 mg | (THC and CBD = | | | | | |
| Weight/tablet = | Mannitol = 577 mg | TBD) | | | | | |
| ~103.1 mg) | Mg stearate = 9 mg | Mannitol = 56.4 mg | | | | | |
| | Kollidon CL ® = | Mg stearate = 0.8 mg | | | | | |
| | 315 mg | Kollidon CL ® = | | | | | |
| | Citric acid = 19 mg | 30.8 mg | | | | | |
| | NaHCO3 = 25 mg | Citric acid = 1.8 mg | | | | | |
| | | NaHCO3 = 2.4 mg | | | | | |
| 59B | | Resin = 9.4 mg | 5 | 0.7 | 4.1 | 23 | |
| (cream tablets; | | (THC and CBD = | | | | | |
| Weight/tablet = | | TBD) | | | | | |
| ~101.6 mg) | | Mannitol = 56.4 mg | | | | | |
| | | Mg stearate = 0.8 mg | | | | | |
| | | Kollidon CL ® = | | | | | |
| | | 30.8 mg | | | | | |
| | | Citric acid = 1.8 mg | | | | | |
| | | NaHCO3 = 2.4 mg | | | | | |

### Experiment 23: Rapidly Disintegrating Sublingual Tablet Formulations Comprising Decarboxylated cannabis Resin from Strain 1 (THC: 7.18%/CBD: 8.6% in the Plant Material; THC: 31.7%/CBD: 46.1% and Other Phytochemicals in the Resin)

Formulation 60-63: (The RDT formula with an average of best/acceptable disintegration times (<30 sec), i.e., Formulations 44, and 54A and the procedure to make the same)

Formulations 60-63 were made according to the procedure used for making Formulation 44.

TABLE 63
Components and properties of sublingual tablet Formulations 60A, 60B, 61A,
61B, 62A, 62B, 63A and 63B; physical properties measured on day 1

| | | Weights of resin | | | | Disintegration | |
|---|---|---|---|---|---|---|---|
| | Total amounts | and | # of | | | time(s) in 1 mL | |
| | of resin and | excipients/tablet | tablets | Friability | Hardness | | Artificial |
| Formulation | excipients (mg) | (mg) | made | (%) | (N) | PBS | saliva |
| 60A | Resin = 102 mg | Resin = 9.3 mg | 5 | 0.20 | 3.6 | 11 | 139 |
| (cream tablets; | Mannitol = 612 mg | (2.6 mg THC; | | | | | |
| Weight/tablet = | Mg stearate = 9 mg | 3.5 mg CBD) | | | | | |
| ~96.8 mg) | Kollidon CL ® = | Mannitol = 56.0 mg | | | | | |
| | 334 mg | Mg stearate = 0.8 mg | | | | | |
| | | Kollidon CL ® = | | | | | |
| | | 30.6 mg | | | | | |
| 60B | | Resin = 9.3 mg | 5 | 0.21 | 5.3 | 11 | 128 |
| (cream tablets; | | (2.6 mg THC; | | | | | |
| Weight/tablet = | | 3.5 mg CBD) | | | | | |
| ~96.2 mg) | | Mannitol = 55.7 mg | | | | | |
| | | Mg stearate = 0.8 mg | | | | | |
| | | Kollidon CL ® = | | | | | |
| | | 30.4 mg | | | | | |
| 61A | Resin = 116.9 mg | Resin = 9.3 mg | 6 | 0 | 10.8 | 17 | 283 |
| (cream tablets; | Mannitol = 701 mg | (3.0 mg THC; | | | | | |
| Weight/tablet = | Mg stearate = 11 mg | 3.6 mg CBD) | | | | | |
| ~96.5 mg) | Kollidon CL ® = | Mannitol = 55.9 mg | | | | | |
| | 383 mg | Mg stearate = 0.8 mg | | | | | |
| | | Kollidon CL ® = | | | | | |
| | | 30.5 mg | | | | | |
| 61B | | | 6 | 0.1 | 8.8 | 23 | 278 |
| (cream tablets; | | | | | | | |
| Weight/tablet = | | | | | | | |
| ~96.5 mg) | | | | | | | |
| 62A | Resin = 174.2 mg | Resin = 9.3 mg | 9 | 0.1 | 2.4 | 13 | 104 |
| (cream tablets; | Mannitol = 1044 mg | (3.0 mg THC; | | | | | |
| Weight/tablet = | Mg stearate = 16 mg | 3.6 mg CBD) | | | | | |
| ~96.5 mg) | Kollidon CL ® = | Mannitol = 55.9 mg | | | | | |
| | 570 mg | Mg stearate = 0.8 mg | | | | | |
| | | Kollidon CL ® = | | | | | |
| | | 30.5 mg | | | | | |
| 62B | | Kollidon CL ® = | 9 | 0.15 | 2.8 | 13 | 116 |
| (cream tablets; | | 30.5 mg | | | | | |
| Weight/tablet = | | | | | | | |
| ~96.6 mg) | | | | | | | |
| 63A | Resin = 104.5 mg | Resin = 9.3 mg | 5 | 0.7 | 1.4 | 11 | 80 |
| (cream tablets; | Mannitol = 629 mg | (3.0 mg THC; | | | | | |
| Weight/tablet = | Mg stearate = 9 mg | 3.6 mg CBD) | | | | | |
| ~96.4 mg) | Kollidon CL ® = | Mannitol = 55.8 mg | | | | | |
| | 344 mg | Mg stearate = 0.8 mg | | | | | |
| | | Kollidon CL ® = | | | | | |
| | | 30.5 mg | | | | | |
| 63B | | Resin = 9.3 mg | 5 | 0.5 | 2.7 | 11 | 123 |
| (cream tablets; | | (3.0 mg THC; | | | | | |
| Weight/tablet = | | 3.6 mg CBD) | | | | | |
| ~95.7 mg) | | Mannitol = 55.4 mg | | | | | |
| | | Mg stearate = 0.8 mg | | | | | |
| | | Kollidon CL ® = | | | | | |
| | | 30.2 mg | | | | | |

General procedure for herein reported sublingual RDT formulations, to test the suitability of F-Melt® in the formulation and comparison to mannitol-only formulation (Tables 64-66), for Experiments 24, 25 and 26:
1. A 100 mg/mL ethanolic solution of resin was prepared and divided into two equal portions into two vials
2. A solution of mannitol in 4 mL water was added to one vial and a suspension of F-Melt® Type C (where the formulation indicated, based on Tables 64-66) in 4 mL water was added to other vial
3. Added 3 more mL of water to each vial
4. Lyophilized all vials for 43 hrs
5. Added other excipients to each vial in quantities as shown in Tables 64-66.
6. Triturated in a porcelain mortar with a pestle until having a homogeneous solid solution
7. Weighed mixture for each tablet and load into a 10 mm die
8. Punched under 0.1 (A) or 0.4 (B) ton pressure, using Specac® manual hydraulic press
9. Collected tablets in 20 mL vials

### Experiment 24: Rapidly Disintegrating Sublingual Tablet Formulations Comprising Decarboxylated cannabis Resin from Strain 3 (THC: 18.6%/CBD: 0% in the Plant Material; THC: 49.8%/CBD: <1% and Other Phytochemicals in the Resin)

TABLE 64
Components and properties of sublingual tablet Formulations 64A, 64B, 65A,
65B, 66A, 66B, 67A and 67B; physical properties measured on day 0

| | | Weights of resin | | | | Disintegration | |
|---|---|---|---|---|---|---|---|
| | Total amounts | and | # of | | | time(s) in 1 mL^{b} | |
| | of resin and | excipients/tablet | tablets | Friability^{a} | Hardness | | Artificial |
| Formulation | excipients (mg) | (mg) | made | (%) | (N) | PBS | saliva |
| 64A | Resin (Strain 3) = | Resin = 11.1 mg | 8 | 0.26 | 9.8 ± 0.6 | 33 | 224 |
| (cream tablets; | 197.4 mg | (5.5 mg THC) | | | | | |
| Weight/tablet = | Mannitol = 1184 mg | Mannitol = 66.4 mg | | | | | |
| ~96.1 mg) | MCC = 178 mg | MCC = 10.0 mg | | | | | |
| | Mg stearate = 9 mg | Mg stearate = 0.5 mg | | | | | |
| | Kollidon CL ® = | Kollidon CL ® = | | | | | |
| | 146 mg | 8.2 mg | | | | | |
| 64B | | | 8 | 0.36 | 9.7 ± 0.4 | 31 | 191 |
| (cream tablets; | | | | | | | |
| Weight/tablet = | | | | | | | |
| ~96.2 mg) | | | | | | | |
| 65A | Resin (Strain 3) = | Resin = 9.6 mg | 6 | 0 | 5.3 | 11 | 61 |
| (cream tablets; | 147.7 mg | (4.7 mg THC) | | | | | |
| Weight/tablet = | Mannitol = 886 mg | Mannitol = 57.3 mg | | | | | |
| ~99.0 mg) | Mg stearate = 13 mg | Mg stearate = 0.9 mg | | | | | |
| | Kollidon CL ® = | Kollidon CL ® = | | | | | |
| | 484 mg | 31.3 mg | | | | | |
| 65B | | Resin = 9.5 mg | 6 | 4.3 | 5.7 | 10 | 79 |
| (cream tablets; | | (4.7 mg THC) | | | | | |
| Weight/tablet = | | Mannitol = 56.7 mg | | | | | |
| ~98.0 mg) | | Mg stearate = 0.9 mg | | | | | |
| | | Kollidon CL ® = | | | | | |
| | | 31.0 mg | | | | | |
| 66A | Resin (Strain 3) = | Resin = 8.2 mg | 5 | 2.1 | 8.3 | 11 | 60 |
| (cream tablets; | 106 mg | (4.1 mg THC) | | | (8.7, 7.9) | | |
| Weight/tablet = | Mannitol = 612 mg | Mannitol = 47.3 mg | | | | | |
| ~96.3 mg) | Mg stearate = 9 mg | Mg stearate = 0.7 mg | | | | | |
| | Neusilin ® UFL2= | Neusilin ® UFL2= | | | | | |
| | 184 mg | 14.2 mg | | | | | |
| | Kollidon CL ® = | Kollidon CL ® = | | | | | |
| | 334 mg | 25.8 mg | | | | | |
| 66B | | Resin = 8.4 mg | 5 | 1.0 | 13.7 | 13 | 69 |
| (cream tablets; | | (4.2 mg THC) | | | (14.6, 12.7) | | |
| Weight/tablet = | | Mannitol = 48.3 mg | | | | | |
| ~98.2 mg) | | Mg stearate = 0.7 mg | | | | | |
| | | Neusilin ® UFL2= | | | | | |
| | | 14.5 mg | | | | | |
| | | Kollidon CL ® = | | | | | |
| | | 26.4 mg | | | | | |
| 67A | Resin (Strain 3) = | Resin = 9.5 mg | 5 | 2.1 | 49 | 265 | >6 mins |
| (cream tablets; | 106 mg | (4.8 mg THC) | | | (4.0, 5.8) | | |
| Weight/tablet = | Mg stearate = 5 mg | Mg stearate = 0.5 mg | | | | | |
| ~95.5 mg) | F-Melt ® Type C = | F-Melt ® Type C = | | | | | |
| | 612 + 286 mg | 80.7 mg | | | | | |
| | Kollidon CL ® = | Kollidon CL ® = | | | | | |
| | 53 mg | 4.8 mg | | | | | |
| 67B | | Resin = 9.6 mg | 5 | 0 | 8.5 | 270 | >6 mins |
| (cream tablets; | | (4.8 mg THC) | | | (6.2, 10.8) | | |
| Weight/tablet = | | Mg stearate = 0.5 mg | | | | | |
| ~96.2 mg) | | F-Melt ® Type C = | | | | | |
| | | 81.3 mg | | | | | |
| | | Kollidon CL ® = | | | | | |
| | | 4.8 mg | | | | | |

^{a}The same tablets were used for friability first then hardness; N = 2 tablets
^{b}N = 1 tablet

### Experiment 25: Rapidly Disintegrating Sublingual Tablet Formulations Comprising Decarboxylated cannabis Resin from Strain 5 (THC: ˜36%/CBD: ˜60% and Other Phytochemicals in the Resin)

TABLE 65
Components and properties of sublingual tablet Formulations 68A, 68B, 69A,
69B, 70A, 70B, 71A and 71B; physical properties measured on day 0

| | | Weights of resin | | | | Disintegration | |
|---|---|---|---|---|---|---|---|
| | Total amounts | and | # of | | | time(s) in 1 mL^{b} | |
| | of resin and | excipients/tablet | tablets | Friability^{a} | Hardness | | Artificial |
| Formulation | excipients (mg) | (mg) | made | (%) | (N) | PBS | saliva |
| 68A | Resin (Strain 5) = | Resin = 9.4 mg | 5 | 0.47 | 11.7 ± 5.9 | 40 | >6 mins |
| (beige tablets; | 102.5 mg | (3.4 mg THC; | | | | | |
| Weight/tablet = | Mannitol = 612 mg | 5.6 mg CBD) | | | | | |
| ~96.6 mg) | MCC = 183 mg | Mannitol = 55.9 mg | | | | | |
| | Mg stearate = 9 mg | MCC = 16.7 mg | | | | | |
| | Kollidon CL ® = | Mg stearate = 0.8 mg | | | | | |
| | 151 mg | Kollidon CL ® = | | | | | |
| | | 13.8 mg | | | | | |
| 68B | | Resin = 3.3 mg | 5 | 0.16 | 10.0 ± 2.9 | 45 | >6 mins |
| (beige tablets; | | (3.4 mg THC; | | | | | |
| Weight/tablet = | | 5.6 mg CBD) | | | | | |
| ~96.4 mg) | | Mannitol = 55.8 mg | | | | | |
| | | MCC = 16.7 mg | | | | | |
| | | Mg stearate = 0.8 mg | | | | | |
| | | Kollidon CL ® = | | | | | |
| | | 13.8 mg | | | | | |
| 69A | Resin (Strain 5) = | Resin = 10.4 mg | 5 | 2.4 | 6.1 | 40 | >6 mins |
| (white tablets; | 113.6 mg | (3.7 mg THC; | | | | | |
| Weight/tablet = | Mannitol = 682 mg | 6.2 mg CBD) | | | | | |
| ~107.6 mg) | Mg stearate = 10 mg | Mannitol = 62.3 mg | | | | | |
| | Kollidon CL ® = | Mg stearate = 0.9 mg | | | | | |
| | 372 mg | Kollidon CL ® = | | | | | |
| | | 34.0 mg | | | | | |
| 69B | | Resin = 10.4 mg | 5 | 5.1 | 5.1 | 62 | >6 mins |
| (white tablets; | | (3.7 mg THC; | | | | | |
| Weight/tablet = | | 6.2 mg CBD) | | | | | |
| ~107.3 mg) | | Mannitol = 62.1 mg | | | | | |
| | | Mg stearate = 0.9 mg | | | | | |
| | | Kollidon CL ® = | | | | | |
| | | 33.9 mg | | | | | |
| 70A | Resin (Strain 5) = | Resin = 8.5 mg | 5 | Failed | 3.8 | 19 | 80 |
| (white tablets; | 115 mg | (3.0 mg THC; | | (both) | (1 tablet) | | |
| Weight/tablet = | Mannitol = 690 mg | 5.1 mg CBD) | | | | | |
| ~102.8 mg) | Mg stearate = 10 mg | Mannitol = 50.7 mg | | | | | |
| | Neusilin ® UFL2 = | Mg stearate = 0.8 mg | | | | | |
| | 207 mg | Neusilin ® UFL2 = | | | | | |
| | Kollidon CL ® = | 15.2 mg | | | | | |
| | 377 mg | Kollidon CL ® = | | | | | |
| | | 27.7 mg | | | | | |
| 70B | | Resin = 8.3 mg | 5 | Failed | 7.0 | 17 | 217 |
| (white tablets; | | (3.0 mg THC; | | (both) | (1 tablet) | | |
| Weight/tablet = | | 5.0 mg CBD) | | | | | |
| ~100.9 mg) | | Mannitol = 49.8 mg | | | | | |
| | | Mg stearate = 0.7 mg | | | | | |
| | | Neusilin ® UFL2 = | | | | | |
| | | 14.9 mg | | | | | |
| | | Kollidon CL ® = | | | | | |
| | | 27.2 mg | | | | | |
| 71A | Resin (Strain 5) = | Resin = 9.3 mg | 5 | 0.5 | 2.1 | >6 mins | >6 mins |
| (white tablets; | 115.7 mg | (3.3 mg THC; | | | (1.7, 2.5) | | |
| Weight/tablet = | Mg stearate = 6 mg | 5.6 mg CBD) | | | | | |
| ~95.8 mg) | F-Melt ® Type C = | Mg stearate = 0.5 mg | | | | | |
| | 650 + 362 mg | F-Melt ® Type C = | | | | | |
| | Kollidon CL ® = | 81.2 mg | | | | | |
| | 60 mg | Kollidon CL ® = | | | | | |
| | | 4.8 mg | | | | | |
| 71B | | Resin = 9.4 mg | 5 | 0 | 6.6 | >6 mins | >6 mins |
| (white tablets; | | (3.4 mg THC; | | | (6.8, 6.3) | | |
| Weight/tablet = | | 5.6 mg CBD) | | | | | |
| ~96.7 mg) | | Mg stearate = 0.5 mg | | | | | |
| | | F-Melt ® Type C = | | | | | |
| | | 82.0 mg | | | | | |
| | | Kollidon CL ® = | | | | | |
| | | 4.8 mg | | | | | |

^{a}The same tablets were used for friability first then hardness; N = 2 tablets
^{b}N = 1 tablet

### Experiment 26: Rapidly Disintegrating Sublingual Tablet Formulations Comprising Decarboxylated cannabis Resin from Strain 2 (THC: 0%/CBD: 9% in the Plant Material; THC: 1.9%/CBD: 37.6% and Other Phytochemicals in the Resin)

TABLE 66
Components and properties of sublingual tablet Formulations 72A, 72B, 73A,
73B, 74A, 74B, 75A and 75B; physical properties measured on day 0

| | | Weights of resin | | | | Disintegration | |
|---|---|---|---|---|---|---|---|
| | Total amounts | and | # of | | | time(s) in 1 mL^{b} | |
| | of resin and | excipients/tablet | tablets | Friability^{a} | Hardness | | Artificial |
| Formulation | excipients (mg) | (mg) | made | (%) | (N) | PBS | saliva |
| 72A | Resin (Strain 2) = | Resin = 11.1 mg | 6 | 0.05 | 5.5 ± 0.4 | 30 | 362 |
| (cream tablets; | 155.2 mg | (0.2 mg THC; | | | | | |
| Weight/tablet = | Mannitol = 931 mg | 4.2 mg CBD) | | | | | |
| ~96.3 mg) | MCC = 140 mg | Mannitol = 66.5 mg | | | | | |
| | Mg stearate = 7 mg | MCC = 10.0 mg | | | | | |
| | Kollidon CL ® = | Mg stearate = 0.5 mg | | | | | |
| | 115 mg | Kollidon CL ® = | | | | | |
| 72B | | 8.2 mg | 6 | 0.05 | 8.5 ± 0.7 | 29 | 361 |
| (cream tablets; | | | | | | | |
| Weight/tablet = | | | | | | | |
| ~96.6 mg) | | | | | | | |
| 73A | Resin (Strain 2) = | Resin = 10.1 mg | 4 | 4.9 | 3.7 | 28 | 253 |
| (beige tablets; | 82.2 mg | (0.2 mg THC; | | | | | |
| Weight/tablet = | Mannitol = 493 mg | 4.1 mg CBD) | | | | | |
| ~104.5 mg) | Mg stearate = 7 mg | Mannitol = 60.5 mg | | | | | |
| | Kollidon CL ® = | Mg stearate = 0.9 mg | | | | | |
| | 269 mg | Kollidon CL ® = | | | | | |
| | | 33.0 mg | | | | | |
| 73B | | Resin = 9.5 mg | 3 | 1.0 | 3.7 | 25 | — |
| (beige tablets; | | (0.2 mg THC; | | | | | |
| Weight/tablet = | | 3.8 mg CBD) | | | | | |
| ~98.0 mg) | | Mannitol = 56.7 mg | | | | | |
| | | Mg stearate = 0.9 mg | | | | | |
| | | Kollidon CL ® = | | | | | |
| | | 31.0 mg | | | | | |
| 74A | Resin (Strain 2) = | Resin = 8.3 mg | 4 | Failed | Not done | 18 | 93 |
| (cream tablets; | 75 mg | (0.1 mg THC; | | both | | | |
| Weight/tablet = | Mannitol = 450 mg | 2.9 mg CBD) | | | | | |
| ~100.4 mg) | Mg stearate = 7 mg | Mannitol = 49.5 mg | | | | | |
| | Neusilin ® UFL2 = | Mg stearate = 0.7 mg | | | | | |
| | 135 mg | Neusilin ® UFL2 = | | | | | |
| | Kollidon CL ® = | 14.9 mg | | | | | |
| | 246 mg | Kollidon CL ® = | | | | | |
| | | 27.0 mg | | | | | |
| 74B | | Resin = 8.1 mg | 4 | 2.5 | 12.6 | 13 | 99 |
| (cream tablets; | | (0.1 mg THC; | | | (1 tablet) | | |
| Weight/tablet = | | 2.8 mg CBD) | | | | | |
| ~98.1 mg) | | Mannitol = 48.4 mg | | | | | |
| | | Mg stearate = 0.7 mg | | | | | |
| | | Neusilin ® UFL2 = | | | | | |
| | | 14.5 mg | | | | | |
| | | Kollidon CL ® = | | | | | |
| | | 26.4 mg | | | | | |
| 75A | Resin (Strain 2) = | Resin = 9.2 mg | 3 | 2.0 | 4.9 | 306 | >6 mins |
| (cream tablets; | 75 mg | (0.2 mg THC; | | (1 tablet) | (1 tablet) | | |
| Weight/tablet = | Mg stearate = 2 mg | 3.2 mg CBD) | | | | | |
| ~95.7 mg) | F-Melt ® Type C = | Mg stearate = 0.2 mg | | | | | |
| | 690 mg | F-Melt ® Type C = | | | | | |
| | Kollidon CL ® = | 84.8 mg | | | | | |
| | 12 mg | Kollidon CL ® = | | | | | |
| | | 1.4 mg | | | | | |
| 75B | | Resin = 9.2 mg | 5 | 1.0 | 15.4 | | |
| (cream tablets; | | (0.2 mg THC; | | | (15.8, 15.0) | 295 | >6 mins |
| Weight/tablet = | | 3.2 mg CBD) | | | | | |
| ~96.0 mg) | | Mg stearate = 0.2 mg | | | | | |
| | | F-Melt ® Type C = | | | | | |
| | | 85.1 mg | | | | | |
| | | Kollidon CL ® = | | | | | |
| | | 1.4 mg | | | | | |

^{a}The same tablets were used for friability first then hardness; N = 2 tablets
^{b}N = 1 tablet
—Not enough tablets for disintegration

Based on the disintegration time data obtained from the tablets (Tables 64-66), inclusion of F-Melt® did not yield faster disintegration of the tablets. However, inclusion of Neusilin® UFL2 appears to help with rapid disintegration. Without being bound by theory, it is noted that this pharmaceutical excipient helps as a carrier for solid dispersions, possesses very large specific surface area and has high oil and water adsorption capacity, makes hard tablets at low compression forces compared to similar binders, increases the hardness synergy with other filler and binder excipients at low concentrations, as well as helps stabilize moisture sensitive as well as lipophilic active pharmaceutical ingredients.

### Experiment 27: Rapidly Disintegrating Sublingual Tablet Formulations Comprising Decarboxylated cannabis Resin from Strain 6 (THC: 22.5%/CBD: 23.1% in the Resin)

Formulation 64 comprised decarboxylated cannabis resin from Strain 6, containing 22.5% of THC and 23.1% CBD, and was made according to the following procedure:
1. Resin was dissolved in ethanol to give a solution of about 102 mg/mL
2. Divided ethanolic solution into 1 mL portions (102 mg of resin per vial)
3. Mannitol was weighed and dissolved in water (4 mL per 0.612 g), and 4 mL of solution was added to each vial
4. Added 3 more mL of water to each vial
5. Lyophilized all vials for 44 hrs
6. Combined vials and added other excipients in quantities as shown in Table 67
7. Triturated in a porcelain mortar with a pestle until having a homogeneous solid solution
8. Weighed about 96.5 mg of the mixture for each tablet and load into a 10 mm die
9. Punched under 0.4 ton pressure, using Specac® manual hydraulic press
10. Collected tablets in a 20 mL vial and covered with cotton which were then closed
11. Tablets for stability studies were subjected to humidity chamber at 40° C.±2° C. and 75%±5° C. relative humidity for the following time-points: 0, 1, 3 and 6 mths

TABLE 67
Components of sublingual tablet Formulation 64. The resin used
contained 22.5% THC and 23.1% CBD

| | Total amounts of | Weights of resin and | |
|---|---|---|---|
| | resin and excipients | excipients/tablet | # of tablets |
| Formulation | (mg) | (mg) | made |
| 64 | Resin = 0.4021 g | Resin = 9.3 mg | 39 |
| (beige tablets; | Mannitol = 2.413 g | (2.1 mg THC; 2.1 mg CBD) | |
| Weight/tablet = 96.4 mg) | MCC = 0.724 g | Mannitol = 55.9 mg | |
| | Mg stearate = 0.036 g | MCC = 16.8 mg | |
| | Kollidon CL ® = | Mg stearate = 0.8 mg | |
| | 0.595 g | Kollidon CL ® = 13.8 mg | |

TABLE 68
Quality measurements and stability data of the formulated tablets
of Formulation 64 stored at 40° C. and 75% Relative Humidity. See also FIGS. 30A,
30B and 31

| | | | | Disintegration | | | | |
|---|---|---|---|---|---|---|---|---|
| | | | Moisture | time(s) in 1 mL | | | | |
| Time | Friability | Hardness | content | | Artificial | % (w/w) Cannabinoid | | |
| (mths) | (%) | (N) | (%) | PBS | saliva | Δ⁹-THC | CBD | CBN |
| 0 | 0.1 | 5.1 ± 0.5 | 2.7 | 19 ± 3 | 209 | 23.8 ± 1 | 35.7 ± 0.0 | — |
| 1 | 1.5 | 9.6 ± 0.4 | 2.6 | 16.4 ± 4.5 | n.d. | 17.9 ± 0.4 | 35.6 ± 0.3 | 3.4 ± 0.5 |
| 3 | 2.3 | 6.9 ± 0.2 | 2.8 | 15.8 ± 5.5 | n.d. | 16.8 ± 0.8 | 41.2 ± 0.5 | 5.3 ± 1.2 |
| 6 | 2.88 | 6.2 ± 1.0 | 2.1 | 26.1 ± 0.7 | n.d. | 12.93 ± 0.1 | 41.3 ± 0.2 | 4.1 ± 1.2 |

*The same tablet (N = 3) were used for friability and hardness; N = 3 for disintegration in PBS and N = 2 for disintegration in saliva and moisture content
—Not detected
n.d. = not determined

Although the disclosure has been described with reference to certain specific embodiments, various modifications thereof will be apparent to those skilled in the art. Any examples provided herein are included solely for the purpose of illustrating the disclosure and are not intended to limit the disclosure in any way. Any drawings provided herein are solely for the purpose of illustrating various aspects of the disclosure and are not intended to be drawn to scale or to limit the disclosure in any way. The scope of the claims appended hereto should not be limited by the preferred embodiments set forth in the above description, but should be given the broadest interpretation consistent with the present specification as a whole. The disclosures of all prior art recited herein are incorporated herein by reference in their entirety.

We claim:

1. A method for making a rapidly disintegrating sublingual tablet from decarboxylated cannabis resin, wherein the decarboxylated cannabis resin comprises cannabinoids which are at least 50% decarboxylated, the method comprising:
(a) dissolving the decarboxylated cannabis resin in a pharmaceutically acceptable organic solvent to produce a first solution;
(b) dissolving mannitol in a pharmaceutically acceptable polar solvent to produce a second solution;
(c) mixing the first solution of (a) and the second solution of (b);
(d) substantially removing the solvents from the mixture of (c) to produce a powder;
(e) adding one or more pharmaceutically acceptable excipients to the powder of (d) to produce a mixture, solid solution or solid suspension; wherein at least one of the pharmaceutically acceptable excipients is a disintegrant;
(f) triturating or mixing the mixture, solid solution or solid suspension of (e) to produce a homogenous mixture, homogeneous solid solution or homogeneous solid suspension; and
(g) compressing the homogenous mixture, homogeneous solid solution or homogeneous solid suspension of (f) into a tablet using a pressure of up to about 1 ton.

2. The method of claim 1, wherein the cannabinoids are at least 55%, at least 60%, at least 65%, at least 70%, at least 75%, at least 80%, at least 85%, at least 90%, at least 95%, at least 96%, at least 97%, at least 98%, at least 99%, or 100% decarboxylated.

3. The method of claim 1, wherein the ratio of cannabis resin to mannitol is about 1:3 to 1:8.

4. The method of claim 1, wherein the organic solvent of (a) is ethanol; wherein the polar solvent of (b) is water; and wherein the disintegrant of (e) is cross-linked polyvinylpyrrolidone and/or croscarmellose sodium.

5. The method of claim 4, wherein the one or more pharmaceutically acceptable excipients comprise magnesium stearate, cross-linked polyvinylpyrrolidone, and optionally microcrystalline cellulose.

6. The method of claim 1, wherein in step (d) the solvents are substantially removed by lyophilization, spray drying, or fluid bed drying.

7. The method of claim 1, wherein in step (g) the pressure is from about 0.05 ton to about 0.6 ton or from about 0.1 to about 0.4 ton.

8. The method of claim 1, wherein the decarboxylated cannabis resin comprises 0 to 95% Δ⁹-tetrahydrocannabinol (Δ⁹-THC) and 0 to 95% cannabidiol including combinations thereof.

9. The method of claim 1, wherein the resin is about 2-18 wt % of the tablet, the mannitol is about 40-80 wt % of the tablet, the disintegrant is about 10-50 wt % of the tablet, and optionally other excipients are about 0-25 wt % of the tablet.

10. The method of claim 9, wherein the resin is about 7-11 wt % of the tablet, the mannitol is about 50-63 wt % of the tablet, the disintegrant is cross-linked polyvinylpyrrolidone and/or croscarmellose sodium and is about 11-18 wt % of the tablet, and the other excipients are magnesium stearate which is about 0.6-1.1 wt % of the tablet, and microcrystalline cellulose which is about 0-20 wt % of the tablet.

11. The method of claim 1, wherein the rapidly disintegrating tablet disintegrates in less than 60 seconds when contacted with a phosphate buffered saline (PBS); and wherein the tablet has a friability of less than 5%.

12. A rapidly disintegrating sublingual tablet from decarboxylated cannabis resin, comprising cannabinoids which are at least 50% decarboxylated, wherein the tablet further comprises mannitol, a disintegrant, and optionally one or more other pharmaceutically acceptable excipients, wherein the tablet disintegrates in less than 60 seconds when contacted with phosphate buffered saline (PBS) and wherein the tablet has a friability of less than 5%.

13. The tablet of claim 12, wherein the cannabinoids are at least 55%, at least 60%, at least 65%, at least 70%, at least 75%, at least 80%, at least 85%, at least 90%, at least 95%, at least 96%, at least 97%, at least 98%, at least 99%, or 100% decarboxylated.

14. The tablet of claim 12, wherein the ratio of cannabis resin to mannitol is about 1:3 to 1:8.

15. The tablet of claim 12, wherein the disintegrant is cross-linked polyvinylpyrrolidone and/or croscarmellose sodium.

16. The tablet of claim 15, wherein the one or more pharmaceutically acceptable excipients comprise magnesium stearate and optionally microcrystalline cellulose and/or Neusilin™.

17. The tablet of claim 12, wherein the combined total of Δ⁹-tetrahydrocannabinol (Δ⁹-THC) and cannabidiol (CBD) is up to about 18 wt % of the tablet.

18. The tablet of claim 12, wherein the resin is about 2-18 wt % of the tablet, the mannitol is about 40-80 wt % of the tablet, the disintegrant is about 10-50 wt % of the tablet, and optionally the other excipients are about 0-25 wt % of the tablet.

19. The tablet of claim 18, wherein the other excipients are optionally a diluent, a filler, a binding agent, a releasing agent, a lubricant, a flavoring agent, a taste-masking agent, and/or a colorant.

20. Use of a decarboxylated cannabis resin in the manufacture of a rapidly disintegrating sublingual tablet, wherein the resin comprises decarboxylated cannabinoids from a Cannabis spp. plant, such that Δ⁹-tetrahydrocannabinolic acid (Δ⁹-THCA) and cannabidiolic acid (CBDA) cannabinoids are each independently 90%-100% decarboxylated to yield Δ⁹-tetrahydrocannabinol (Δ⁹-THC) and cannabidiol (CBD) respectively in the cannabis resin.
